(19) United States
(12) Patent Application Publication
(10) Pub. No.: US 2025/0297021 A1
(43) Pub. Date: Sep. 25, 2025

(54) BCMA ANTIBODY AND USE THEREOF

(71) Applicant: SHANDONG SIMCERE BIOPHARMACEUTICAL CO., No. 1.,, Yantai, Shandong (CN)

(72) Inventors: Yayuan FU, Shanghai (CN); Shumei YOU, Shanghai (CN); Bowei YIN, Shanghai (CN); Zhuoxiao CAO, Shanghai (CN); Renhong TANG, Shanghai (CN); Jinsheng REN, Nanjing, Jiangsu (CN)

(21) Appl. No.: 18/717,808

(22) PCT Filed: Dec. 8, 2022
(86) PCT No.: PCT/CN2022/137477

§ 371 Date: Jun. 7, 2024

(30) Foreign Application Priority Data
Dec. 9, 2021 (CN) 202111501235.5

(51) Int. Cl. C07K 16/28; G01N 33/68

(52) U.S. Cl. CPC C07K 16/2878; G01N 33/6854; C07K 2317/24; C07K 2317/52; C07K 2317/569; C07K 2317/622; C07K 2317/92; G01N 2333/70578

(57) ABSTRACT

The present invention relates to an antibody against a B-cell maturation antigen (BCMA) and the use thereof. Specifically, disclosed herein are an antibody specifically binding to BCMA or an antigen-binding fragment thereof, and an encoding nucleic acid, an expression vector and an expression cell thereof, a preparation method therefor, a pharmaceutical composition thereof, and the use thereof in the preparation of a pharmaceutical composition for treating diseases, for example, for treating tumors. The present invention has important significance for the development of a therapeutic BCMA antibody drug and a detection reagent.

### CROSS REFERENCE TO RELATED PATENT APPLICATIONS

The present application claims the right of priority for Chinese patent application no. 202111501235.5, entitled “BCMA ANTIBODY AND USE THEREOF” and submitted to the China National Intellectual Property Administration Dec. 9, 2021. The above-mentioned prior application is incorporated into the present application by reference in its entirety.

### TECHNICAL FIELD

The present application relates to the field of biomedicine, and specifically to an anti-BCMA antibody or an antigen-binding fragment thereof and use thereof.

### BACKGROUND

Multiple myeloma (MM) is a malignant plasma cell tumor originating in the bone marrow, is a type of B-cell lymphoma and is also called plasma cell tumor. It is characterized by abnormal proliferation of bone marrow plasma cells accompanied by overproduction of monoclonal immunoglobulin or light chain (M protein). A very small number of patients may have unsecreted MM that does not produce M protein. Multiple myeloma is often accompanied by multiple osteolytic lesions, hypercalcemia, anemia, and renal damage. Since the production of normal immunoglobulins is suppressed, various bacterial infections are prone to occur.

Multiple myeloma accounts for 1% of all tumors and 10%-15% of hematologic malignancies. The ratio of male to female is 1.6:1, and most patients are >40 years old. The treatment of multiple myeloma includes chemotherapy, hematopoietic stem cell transplantation, etc. Among them, immunomodulators represented by lenalidomide and protease inhibitors represented by bortezomib, used either alone or in combination, have shown good efficacy, and have become conventional treatment methods for patients with multiple myeloma. However, multiple myeloma is still considered an incurable disease. Current treatments can only relieve the symptoms of multiple myeloma, but cannot completely eliminate the tumor. Almost all patients will eventually relapse. Therefore, there is an urgent need for new treatment options.

B cell maturation antigen (BCMA), also known as CD269 or TNFRSF17, is a member of the tumor necrosis factor receptor superfamily and was first identified in the early 1990s. This receptor is mainly expressed on the surface of mature B lymphocytes and plasma cells, is a marker protein of B lymphocyte maturation and is hardly expressed in other tissue cells. Structurally, BCMA consists of three main domains: an extracellular segment (aa1-54), a transmembrane region (aa55-77) and an intracellular segment (aa78-184). B cell activating factor (BAFF) and proliferation inducing ligand (APRIL) are the main ligands of BCMA, which transmit cell stimulation signals by interacting with BCMA, activate TRAF-dependent NF-KB and JNK pathways and increase the proliferation and survival rate of B cells. BCMA is highly expressed in MM cells and is an ideal antigen target for multiple myeloma. The homology of BCMA between human and monkeys is 88%, so it is difficult to screen cross-binding antibodies.

Treatment therapies targeting BCMA, such as CAR T, bispecific antibodies and ADCs have made important progress and show bright prospects. However, there is still an urgent need to develop a new generation of more efficient BCMA-specific antibodies and biotherapeutic products based on them.

### SUMMARY OF THE INVENTION

The present application discloses an antibody or an antigen-binding fragment thereof that specifically binds to B cell mature antigen (BCMA), a multispecific antigen-binding molecule, a nucleic acid fragment, a vector, a host cell, an immune effector cell, a preparation method, a pharmaceutical composition, a pharmaceutical use, and a method of treating a tumor or a cancer (e.g., B cell lymphoma or multiple myeloma). The antibody can block the binding of BCMA's natural ligands (such as BAFF, APRIL) to BCMA.

In one aspect, the present application provides an antibody or an antigen-binding fragment thereof that specifically binds to B cell maturation antigen (BCMA), wherein the antibody or the antigen-binding fragment thereof comprises a light chain variable region (VL) and a heavy chain variable region (VH), and wherein
(1) the light chain variable region comprises LCDR1, LCDR2 and LCDR3, the LCDR1 has any of a sequence of the following LCDR1, or a sequence with 1, 2, 3 or more amino acid insertions, deletions and/or substitutions compared to the sequence, the LCDR2 has any of a sequence of the following LCDR2, or a sequence with 1, 2, 3 or more amino acid insertions, deletions and/or substitutions compared to the sequence, and the LCDR3 has any of a sequence of the following LCDR3, or a sequence with 1, 2, 3 or more amino acid insertions, deletions and/or substitutions compared to the sequence:

| No. | LCDR1 | LCDR2 | LCDR3 |
|---|---|---|---|
| VL1 | SEQ ID NO: 107 | SEQ ID NO: 108 | SEQ ID NO: 109 |
| VL2 | SEQ ID NO: 110 | SEQ ID NO: 111 | SEQ ID NO: 109 |
| VL3 | SEQ ID NO: 118 | SEQ ID NO: 119 | SEQ ID NO: 120 |
| VL4 | SEQ ID NO: 121 | SEQ ID NO: 122 | SEQ ID NO: 120 |
| VL5 | SEQ ID NO: 129 | SEQ ID NO: 130 | SEQ ID NO: 131 |
| VL6 | SEQ ID NO: 132 | SEQ ID NO: 133 | SEQ ID NO: 131 |
| VL7 | SEQ ID NO: 140 | SEQ ID NO: 141 | SEQ ID NO: 142 |
| VL8 | SEQ ID NO: 143 | SEQ ID NO: 144 | SEQ ID NO: 142 |
| VL9 | SEQ ID NO: 151 | SEQ ID NO: 152 | SEQ ID NO: 153 |
| VL10 | SEQ ID NO: 154 | SEQ ID NO: 155 | SEQ ID NO: 153 |
| VL11 | SEQ ID NO: 162 | SEQ ID NO: 163 | SEQ ID NO: 164 |
| VL12 | SEQ ID NO: 165 | SEQ ID NO: 166 | SEQ ID NO: 164 |
| VL13 | SEQ ID NO: 173 | SEQ ID NO: 174 | SEQ ID NO: 175 |
| VL14 | SEQ ID NO: 176 | SEQ ID NO: 177 | SEQ ID NO: 175 |
| VL15 | SEQ ID NO: 178 | SEQ ID NO: 108 | SEQ ID NO: 109 |
| VL16 | SEQ ID NO: 162 | SEQ ID NO: 163 | SEQ ID NO: 183 |
| VL17 | SEQ ID NO: 162 | SEQ ID NO: 163 | SEQ ID NO: 184 |

and,
(2) the heavy chain variable region comprises HCDR1, HCDR2 and HCDR3, the HCDR1 has any of a sequence of the following HCDR1, or a sequence with 1, 2, 3 or more amino acid insertions, deletions and/or substitutions compared to the sequence, the HCDR2 has any of a sequence of the following HCDR2, or a sequence with 1, 2, 3 or more amino acid insertions, deletions and/or substitutions compared to the sequence, and the HCDR3 has any of a sequence of the following HCDR3, or a sequence with 1, 2, 3 or more amino acid insertions, deletions and/or substitutions compared to the sequence:

| No. | HCDR1 | HCDR2 | HCDR3 |
|---|---|---|---|
| VH1 | SEQ ID NO: 101 | SEQ ID NO: 102 | SEQ ID NO: 103 |
| VH2 | SEQ ID NO: 104 | SEQ ID NO: 105 | SEQ ID NO: 106 |
| VH3 | SEQ ID NO: 112 | SEQ ID NO: 113 | SEQ ID NO: 114 |
| VH4 | SEQ ID NO: 115 | SEQ ID NO: 116 | SEQ ID NO: 117 |
| VH5 | SEQ ID NO: 123 | SEQ ID NO: 124 | SEQ ID NO: 125 |
| VH6 | SEQ ID NO: 126 | SEQ ID NO: 127 | SEQ ID NO: 128 |
| VH7 | SEQ ID NO: 134 | SEQ ID NO: 135 | SEQ ID NO: 136 |
| VH8 | SEQ ID NO: 137 | SEQ ID NO: 138 | SEQ ID NO: 139 |
| VH9 | SEQ ID NO: 145 | SEQ ID NO: 146 | SEQ ID NO: 147 |
| VH10 | SEQ ID NO: 148 | SEQ ID NO: 149 | SEQ ID NO: 150 |
| VH11 | SEQ ID NO: 156 | SEQ ID NO: 157 | SEQ ID NO: 158 |
| VH12 | SEQ ID NO: 159 | SEQ ID NO: 160 | SEQ ID NO: 161 |
| VH13 | SEQ ID NO: 167 | SEQ ID NO: 168 | SEQ ID NO: 169 |
| VH14 | SEQ ID NO: 170 | SEQ ID NO: 171 | SEQ ID NO: 172 |
| VH15 | SEQ ID NO: 101 | SEQ ID NO: 179 | SEQ ID NO: 103 |
| VH16 | SEQ ID NO: 123 | SEQ ID NO: 180 | SEQ ID NO: 125 |
| VH17 | SEQ ID NO: 134 | SEQ ID NO: 181 | SEQ ID NO: 136 |
| VH18 | SEQ ID NO: 145 | SEQ ID NO: 182 | SEQ ID NO: 147 |

In another aspect, the present application provides a multispecific antigen-binding molecule, wherein the multispecific antigen-binding molecule comprises the aforementioned antibody or the antigen-binding fragment thereof, and an antigen-binding molecule binding to an additional antigen other than BCMA or binding to a different BCMA epitope from that of the aforementioned antibody or the antigen-binding fragment thereof.

In another aspect, the present application provides a chimeric antigen receptor (CAR), wherein the chimeric antigen receptor at least comprises a signal peptide, an extracellular antigen-binding domain, a hinge region, a transmembrane domain, and an intracellular signaling domain, the extracellular antigen-binding domain comprises the aforementioned BCMA antibody or the antigen-binding fragment thereof, or the aforementioned multispecific antigen-binding molecule.

In another aspect, the present application provides an immune effector cell, wherein the immune effector cell expresses the aforementioned chimeric antigen receptor, or contains a nucleic acid fragment encoding the aforementioned chimeric antigen receptor.

In another aspect, the present application provides an isolated nucleic acid fragment, wherein the nucleic acid fragment encodes the aforementioned antibody or the antigen-binding fragment thereof, the aforementioned multispecific antigen-binding molecule, or the aforementioned chimeric antigen receptor.

In another aspect, the present application provides a vector, wherein the vector includes the aforementioned nucleic acid fragment.

In another aspect, the present application provides a host cell, wherein the host cell includes the aforementioned vector.

In another aspect, the present application provides a method of preparing the aforementioned antibody or the antigen-binding fragment thereof or the aforementioned multispecific antigen-binding molecule, wherein the method comprises culturing the aforementioned cell, and isolating the antibody, antigen-binding fragment or multispecific antigen-binding molecule expressed by the cell.

In another aspect, the present application provides a method of preparing the aforementioned immune effector cells, wherein the method comprises introducing a nucleic acid fragment encoding the aforementioned CAR into an immune effector cell.

In another aspect, the present application provides a pharmaceutical composition, wherein the pharmaceutical composition comprises the aforementioned antibody or the antigen-binding fragment thereof, the aforementioned multispecific antigen-binding molecule, the aforementioned immune effector cell, the aforementioned nucleic acid fragment, the aforementioned vector, or a product prepared according to the aforementioned method.

In another aspect, the present application provides a method of treating tumors or cancers, wherein the method comprises administering to a subject an effective amount of the aforementioned antibody or the antigen-binding fragment thereof, the aforementioned multispecific antigen-binding molecule, the aforementioned immune effector cell, the aforementioned nucleic acid fragment, the aforementioned vector, or a product prepared according to the aforementioned method or the aforementioned pharmaceutical composition; the tumor or cancer is a tumor or cancer that expresses BCMA.

In another aspect, the present application provides the use of the aforementioned antibody or the antigen-binding fragment thereof, the aforementioned multispecific antigen-binding molecule, the aforementioned immune effector cell, the aforementioned nucleic acid fragment, the aforementioned vector, or a product prepared according to the aforementioned method or the aforementioned pharmaceutical composition in the manufacture of a medicament for the treatment of a tumor or a cancer; the tumor or cancer is a tumor or cancer that expresses BCMA.

In another aspect, the present application provides the aforementioned antibody or the antigen-binding fragment thereof, the aforementioned multispecific antigen-binding molecule, the aforementioned immune effector cell, the aforementioned nucleic acid fragment, the aforementioned vector, or a product prepared according to the aforementioned method or the aforementioned pharmaceutical composition for use in the treatment of a tumor or a cancer; the tumor or cancer is a tumor or cancer that expresses BCMA.

In another aspect, the present application provides a kit, wherein the kit comprises the aforementioned antibody or the antigen-binding fragment thereof, the aforementioned multispecific antigen-binding molecule, the aforementioned immune effector cell, the aforementioned nucleic acid fragment, the aforementioned vector, or a product prepared according to the aforementioned method or the aforementioned pharmaceutical composition.

In another aspect, the present application provides a method of detecting BCMA expression in a biological sample, wherein the method comprises contacting the biological sample with the aforementioned antibody or the antigen-binding fragment thereof under conditions that allow the formation of a complex from the antibody or the antigen-binding fragment thereof and BCMA.

In another aspect, the present application provides the use of the aforementioned antibody or the antigen-binding fragment thereof in preparing a BCMA detection reagent.

The present application provides an antibody or an antigen-binding fragment thereof with high affinity for a BCMA target, which can better block the binding of BCMA to its ligand APRIL, thus providing a better choice for BCMA antibody drugs and cell therapy products, and is of great significance for filling the gap of treatment means for multiple myeloma.

### BRIEF DESCRIPTION OF THE FIGURES

FIG. 1A shows the ELISA detection of the binding activity of control antibody with human BCMA-His protein; FIG. 1B shows the ELISA detection of the binding activity of control antibody with monkey BCMA-His protein.

FIG. 2A shows the FACS results of detecting BCMA expression in H929 cells using REGN5459-hlgG1 and HPN217-hHcAb antibodies; FIG. 2B shows the FACS results of detecting BCMA expression in U266 cells using REGN5459-hlgG1 and HPN217-hHcAb antibodies; FIG. 2C shows the FACS results of detecting BCMA expression quantity in RPMI8226 cells using REGN5459-hlgG1 and HPN217-hHcAb antibodies.

FIG. 3 shows the FACS results of detecting BCMA expression in Flp-inCHO-human BCMA cells REGN5459-hlgG1 antibody;

FIG. 4 shows the FACS results of detecting BCMA expression in Flp-inCHO-monkey BCMA cells REGN5459-hlgG1 antibody;

FIG. 5A shows the ELISA detection of the binding of serum antibody to human BCMA-his protein after immunization of G1 mice with the protein; FIG. 5B shows the ELISA detection of the binding of serum antibody to human BCMA-his protein after immunization of G2 mice with the protein.

FIG. 6A shows the ELISA detection of the binding of serum antibody to monkey BCMA-his protein after immunization of G1 mice with the protein; FIG. 6B shows the ELISA detection of the binding of serum antibody to monkey BCMA-his protein after immunization of G2 mice with the protein.

FIG. 7A-FIG. 7B shows the ELISA detection of the binding activity of chimeric antibody with human BCMA-his protein.

FIG. 8A-FIG. 8B shows the ELISA detection of the binding activity of chimeric antibody with monkey BCMA-his protein.

FIG. 9A-FIG. 9B shows the cell-based ELISA detection of the binding activity of chimeric antibody with Flp-inCHO-human BCMA cell.

FIG. 10A-FIG. 10B shows the FACS detection of the binding activity of chimeric antibody with H929 tumor cell.

FIG. 11A-FIG. 11B shows the FACS detection of the binding activity of chimeric antibody with U266 tumor cell.

FIG. 12A-FIG. 12B shows the FACS detection of the binding activity of chimeric antibody with RPMI8226 tumor cell.

FIGS. 13A to 13C show the ligand binding competition ELISA detection of the blocking effect of chimeric antibody on the binding of ligand APRIL to human BCMA protein.

FIG. 14 shows the ELISA detection of the binding activity of humanized antibody with human BCMA-his protein.

FIG. 15 shows the ELISA detection of the binding activity of humanized antibody with monkey BCMA-his protein.

FIG. 16 shows the FACS detection of the binding activity of humanized antibody and U266 tumor cell.

FIG. 17 shows the FACS detection of the binding activity of humanized antibody and H929 tumor cell.

FIG. 18 shows the ligand binding competition ELISA detection of the blocking effect of humanized antibody on the binding of ligand APRIL to human BCMA protein.

### DETAILED DESCRIPTION OF THE INVENTION

### Definition and Description of Terminology

Unless defined otherwise in the present application, scientific and technical terms related to the present application shall have the meanings understood by one of ordinary skill in the art.

In addition, unless otherwise specified herein, terms in the singular form herein shall include that in the plural form, and terms in the plural form shall include that in the singular form. More specifically, as used in the description and the appended claims, the singular forms “a/an” and “this” include plural referents, unless otherwise clearly stated.

The terms “include”, “comprise” and “have” are used interchangeably herein and are intended to indicate the inclusiveness of the solution, meaning that the solution can have other elements than those listed. Furthermore, it should be understood that the description of “include”, “comprise” and “have” as used herein also provides the solution of “consist of”.

The term “and/or” as used herein includes the meanings of “and”, “or” and “all or any other combination of elements linked by the term”.

The term “BCMA” herein is the full name of B cell maturation antigen, which is a member of the tumor necrosis factor receptor family. BCMA is mainly expressed on the surface of late B cells, short-lived proliferative plasma cells and long-lived plasma cells, and is not expressed in naive B cells, CD34-positive hematopoietic stem cells and other normal tissue cells, but is highly expressed in MM cells. BCMA plays a key role in the survival, proliferation, metastasis and drug resistance of MM cells by mediating downstream signaling pathways, thereby being an ideal target antigen for the treatment of MM.

The term “antigen-binding molecule” is used herein in the broadest sense and refers to a molecule specifically binding to an antigen. Exemplarily, the antigen-binding molecule includes, but is not limited to, an antibody or an antibody mimetic. The “antibody mimetic” refers to an organic compound or a binding domain that can specifically bind to an antigen, but is not structurally related to an antibody. Exemplarily, the antibody mimetic includes, but is not limited to, affibody, affitin, affilin, a designed ankyrin repeat protein (DARPin), a nucleic acid aptamer or a Kunitz-type domain peptide.

The term “antibody” is used herein in the broadest sense and refers to a polypeptide or a combination of polypeptides comprising sufficient sequences from a heavy chain variable region of an immunoglobulin and/or sufficient sequences from a light chain variable region of an immunoglobulin to be able to specifically bind to an antigen. The “antibody” herein encompasses various forms and various structures as long as they exhibit the desired antigen-binding activity. The “antibody” herein includes an alternative protein scaffold or artificial scaffold with grafted complementarity determining regions (CDRs) or CDR derivatives. Such scaffolds include antibody-derived scaffolds comprising mutations introduced, e.g., to stabilize the three-dimensional structure of the antibody, and fully synthetic scaffolds comprising, e.g., biocompatible polymers. See, for example, Korndorfer et al., 2003, Proteins: Structure, Function, and Bioinformatics, 53 (1): 121-129 (2003); Roque et al., Biotechnol. Prog. 20:639-654 (2004). Such scaffolds may also include non-antibody-derived scaffolds, for example, scaffold proteins known in the art which can be used for grafting CDRs, including but not limited to tenascin, fibronectin, a peptide aptamer, etc.

The term “antibody” herein includes an intact antibody and any antigen-binding fragment (i.e., “antigen-binding moiety”) or single chain thereof. The “antibody” refers to a glycoprotein comprising at least two heavy (H) chains and two light (L) chains interconnected by a disulfide bond, or an antigen-binding moiety thereof. Each heavy chain consists of a heavy chain variable region (abbreviated herein as VH) and a heavy chain constant region. The heavy chain constant region consists of three domains, CH1, CH2 and CH3. Each light chain consists of a light chain variable region (abbreviated herein as VL) and a light chain constant region. The light chain constant region consists of one domain, CL. The VH and VL regions can be further subdivided into hypervariable regions known as complementarity determining regions (CDRs), which are interspersed among more conserved regions known as framework regions (FRs). Each VH and VL consists of three CDRs and four FRs, which are arranged in the following order from amino terminus to carboxyl terminus: FR1, CDR1, FR2, CDR2, FR3, CDR3, FR4. The variable regions of the heavy and light chains contain a binding domain that can interact with an antigen. The constant region of an antibody can mediate the binding of an immunoglobulin to a host tissue or factor, and the host tissue or factor includes various cells of the immune system (e.g., effector cells) and a first component of the classical complement system (Clq). Since the amino acid composition and arrangement sequence of the heavy chain constant region of an immunoglobulin are different, the antigenicity of the immunoglobulin is also different. Accordingly, “immunoglobulin” herein can be divided into five classes, or isotypes of immunoglobulins, namely IgM, IgD, IgG, IgA and IgE, and the corresponding heavy chains thereof are a μ chain, a δ chain, a γ chain, an α chain and an ε chain, respectively. The same class of Ig can also be divided into different subclasses according to the differences in the amino acid composition of the hinge region thereof and the number and position of heavy chain disulfide bonds. For example, IgG can be divided into IgG1, lgG2, lgG3 and IgG4, and IgA can be divided into IgA1 and IgA2. The light chains are divided into a κ chain or a λ chain by the difference in the constant region. Each Ig class of the five Ig classes can have either a κ chain or a λ chain.

“Antibody” herein also includes an antibody that does not comprise light chains, for example, heavy-chain antibodies (HCAbs) produced from camelids, such as Camelus dromedarius, Camelus bactrianus, Lama glama, Lama guanicoe and Vicugna pacos, and Ig new antigen receptors (IgNARs) found in Chondrichthyes such as sharks.

The term “antibody” herein may be derived from any animal, including but not limited to humans and non-human animals selected from primates, mammals, rodents and vertebrates, such as Camelidae, Lama glama, Lama guanicoe, Vicugna pacos, sheep, rabbits, mice, rats or Chondrichthyes (such as sharks).

The term “heavy chain antibody” herein refers to an antibody that lacks light chains of a conventional antibody. The term specifically includes, but is not limited to, a homodimeric antibody comprising a VH antigen-binding domain and CH2 and CH3 constant domains in the absence of a CH1 domain.

The terms “VHH domain”, “nanobody” and “single domain antibody” (sdAb) herein have the same meaning and are used interchangeably, and refer to a single domain antibody consisting of only one heavy chain variable region constructed by cloning the variable region of a heavy chain antibody, which is the smallest antigen-binding fragment with full functionality. Generally, a single domain antibody consisting of only one heavy chain variable region is constructed by obtaining a heavy chain antibody that naturally lacks light chains and heavy chain constant region 1 (CH1), and then cloning the heavy chain variable region of the antibody.

Further descriptions of “heavy chain antibody” and “single domain antibody”, and “VHH domain” and “nanobody” can be found in Hamers-Casterman et al., Nature. 1993; 363; 446-8; Muyldermans' review article (Reviews in Molecular Biotechnology 74:277-302, 2001); and the following patent applications mentioned as general background art: WO 94/04678, WO 95/04079 and WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825, and other prior art mentioned in these applications.

The term “multispecific” herein refers to the ability of an antibody or an antigen-binding fragment thereof to bind to, for example, different antigens or at least two different epitopes on the same antigen. Therefore, the terms such as “bispecific”, “trispecific” and “tetraspecific” refer to the number of different epitopes to which an antibody can bind. For example, a conventional monospecific IgG-type antibody has two identical antigen-binding sites (paratopes) and thus can only bind to the same epitope (rather than bind to different epitopes). In contrast, a multispecific antibody has at least two different types of paratopes/binding sites and thus can bind to at least two different epitopes. As described herein, “complementarity determining region” refers to an antigen-binding site of an antibody. Furthermore, single “specific” may refer to one, two, three or more identical complementarity determining regions in a single antibody (the actual number of complementarity determining regions/binding sites in a single antibody molecule is referred to as “valent”). For example, a single natural IgG antibody is monospecific and bivalent because it has two identical paratopes. Accordingly, a multispecific antibody comprises at least two (different) complementarity determining regions/binding sites. Therefore, the term “multispecific antibody” refers to an antibody that has more than one paratope and has the ability to bind to two or more different epitopes. The term “multispecific antibody” particularly includes the bispecific antibody as defined above, and generally also includes a protein, e.g., an antibody or a scaffold specifically binding to three or more different epitopes, i.e., an antibody having three or more paratopes/binding sites.

The term “valence” herein refers to the presence of a defined number of binding sites in an antibody/an antigen-binding molecule. Therefore, the terms “monovalent”, “bivalent”, “tetravalent” and “hexavalent” indicate the presence of one binding site, two binding sites, four binding sites and six binding sites in an antibody/antigen-binding molecule, respectively.

The “full-length antibody”, “complete antibody” and “intact antibody” are used interchangeably herein and mean that they have a structure substantially similar to that of a natural antibody.

The “antigen-binding fragment” and “antibody fragment” are used interchangeably herein, which do not possess the full structure of an intact antibody, and only comprise a part of an intact antibody or a variant of the part, wherein the part of the intact antibody or the variant of the part has the ability to bind to an antigen. Exemplarily, the “antigen-binding fragment” or “antibody fragment” herein includes, but is not limited to, Fab, F(ab′) 2, Fab′, Fab′-SH, Fd, Fv, scFv, diabody and a single domain antibody.

The term “chimeric antibody” herein refers to an antibody that has a variable sequence of an immunoglobulin from a source organism (such as a rat, a mouse, a rabbit or a llama) and a constant region of an immunoglobulin from a different organism (such as human). Methods for producing a chimeric antibody are known in the art. See, for example, Morrison, 1985, Science 229 (4719): 1202-7; Oi et al., 1986, Bio Techniques 4:214-221; and Gillies et al., 1985 J Immunol Methods 125:191-202; which are incorporated herein by reference.

The term “humanized antibody” herein refers to a non-human antibody that has been genetically engineered, with amino acid sequences modified to improve the homology with the sequences of a human antibody. Generally speaking, all or part of the CDR regions of a humanized antibody come from a non-human antibody (a donor antibody), and all or part of the non-CDR regions (for example, a variable region FR and/or a constant region) come from a human immunoglobulin (a receptor antibody). A humanized antibody usually retains or partially retains the expected properties of a donor antibody, including but not limited to, antigen specificity, affinity, reactivity, ability to enhance immune cell activity or ability to enhance immune response, etc.

The term “fully human antibody” herein refers to an antibody having variable regions in which both the FRs and CDRs are derived from human germline immunoglobulin sequences. Furthermore, if the antibody comprises a constant region, the constant region also is derived from human germline immunoglobulin sequences. The “fully human antibody” herein may include amino acid residues not encoded by human germline immunoglobulin sequences (for example, mutations introduced by random or site-specific mutagenesis in vitro or by somatic mutation in vivo). However, the “fully human antibody” herein does not include an antibody in which CDR sequences derived from the germline of another mammalian species (e.g., a mouse) have been grafted onto human framework sequences.

The term “variable region” herein refers to a region in the heavy or light chain of an antibody that is involved in enabling the antibody to bind to an antigen. “Heavy chain variable region”, “VH” and “HCVR” are used interchangeably, and “light chain variable region”, “VL” and “LCVR” are used interchangeably. The variable domains of the heavy and light chains of a natural antibody generally have similar structures, and each domain comprises four conserved framework regions (FRs) and three hypervariable regions (HVRs). See, for example, Kindt et al., Kuby Immunology, 6th ed., W.H. Freeman and Co., p. 91 (2007). A single VH or VL domain may be sufficient to confer antigen binding specificity.

The terms “complementarity determining region” and “CDR” are used interchangeably herein and generally refer to hypervariable regions (HVRs) found in both light and heavy chain variable domains. The more conservative portion of a variable domain is called the framework region (FR). As understood in the art, the amino acid positions representing the hypervariable region of an antibody may vary according to the context and various definitions known in the art. Some positions within variable domains can be considered heterozygous hypervariable positions because these positions can be considered to be within the hypervariable regions under one set of criteria (such as IMGT or KABAT) but outside the hypervariable regions under a different set of criteria (such as KABAT or IMGT). One or more of these positions may also be found in extended hypervariable regions. The present application includes an antibody comprising modifications in these heterozygous hypervariable positions. The heavy chain variable region CDR can be abbreviated as HCDR, and the light chain variable region CDR can be abbreviated as LCDR. The variable domains of a natural heavy chain and light chain each comprise four framework regions predominantly in a sheet configuration, which are linked by three CDRs (CDR1, CDR2 and CDR3) that form a loop linking the sheet structure, and in some cases form part of the sheet structure. The CDRs in each chain are closely held together in order of FR1-CDR1-FR2-CDR2-FR3-CDR3-FR4 by the FR region, and together with the CDRs from other antibody chains, contribute to the formation of an antigen-binding site of an antibody (see Kabat et al., Sequences of Proteins of Immunological Interest, National Institute of Health, Bethesda, Md. 1987; which is incorporated herein by reference).

For a further description of CDR, with reference to Kabat et al., J. Biol. Chem., 252:6609-6616 (1977); Kabat et al., United States Department of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Pluckthun, J. Mol. Biol., 309:657-670 (2001). The “CDRs” herein can be marked and defined by well-known methods in the art, including but not limited to Kabat numbering system, Chothia numbering system or IMGT numbering system. The tool websites used include, but are not limited to, AbRSA website (http://cao.labshare.cn/AbRSA/cdrs.php), abYsis website (www.abysis.org/abysis/sequence_input/key_annotation/key_annotation.cgi) and IMGT website (http://www.imgt.org/3Dstructure-DB/cgi/DomainGapAlign. cgi #results). The CDRs herein include overlaps and subsets of amino acid residues defined in different ways.

The term “Kabat numbering system” herein generally refers to the immunoglobulin alignment and numbering system proposed by Elvin A. Kabat (see, for example, Kabat et al, Sequences of Proteins of Immunological Interest, 5th Ed. Public Health Service, National Institutes of Health, Bethesda, Md., 1991).

The term “Chothia numbering system” herein generally refers to the immunoglobulin numbering system proposed by Chothia et al., which is a classical rule for identifying the boundaries of CDR regions based on the location of structural loop regions (see, for example, Chothia & Lesk (1987) J. Mol. Biol. 196:901-917; Chothia et al. (1989) Nature 342:878-883).

The term “IMGT numbering system” herein generally refers to the numbering system based on The International ImMunoGeneTics Information System (IMGT) proposed by Lefranc et al., see Lefranc et al., Dev. Comparat. Immunol.27:55-77, 2003.

The term “heavy chain constant region” herein refers to the carboxyl terminus portion of an antibody heavy chain, which is not directly involved in the binding of an antibody to an antigen, but exhibits an effector function, such as an interaction with an Fc receptor, and has a more conservative amino acid sequence relative to the variable domain of an antibody. The “heavy chain constant region” is selected from a CH1 domain, a hinge region, a CH2 domain, a CH3 domain, or a variant or fragment thereof. The “heavy chain constant region” includes a “full-length heavy chain constant region” and a “heavy chain constant region fragment”, the former has a structure substantially similar to that of a natural antibody constant region, while the latter only includes “a portion of the full-length heavy chain constant region”. Exemplarily, a typical “full-length antibody heavy chain constant region” consists of CH1 domain-hinge region-CH2 domain-CH3 domain, which also includes a CH4 domain when an IgE antibody is referred to, and does not include the CH1 domain when a heavy chain antibody is referred to. Exemplarily, a typical “heavy chain constant region fragment” is selected from Fc or a CH3 domain.

The term “light chain constant region” herein refers to the carboxyl terminus portion of an antibody light chain, which is not directly involved in the binding of an antibody to an antigen, and the light chain constant region is selected from a constant K domain or a constant A domain.

The term “Fc region” herein is used to define the C-terminal region of an antibody heavy chain that contains at least a portion of the constant region. The term includes a native sequence Fc region and a variant Fc region. Exemplarily, human IgG heavy chain Fc region can extend from Cys226 or Pro230 to the carboxyl terminus of the heavy chain. However, an antibody generated from a host cell may undergo post-translational cleavage whereby one or more, particularly one or two amino acids are cleaved off from the C-terminus of the heavy chain. Therefore, by expression of a specific nucleic acid molecule encoding a full-length heavy chain, the antibody generated from a host cell can include the full-length heavy chain, or a cleavage variant of the full-length heavy chain. This may be the case when the last two C-terminal amino acids of the heavy chain are glycine (G446) and lysine (K447, numbering according to the Kabat EU index). Therefore, the C-terminal lysine (Lys447), or the C-terminal glycine (Gly446) and lysine (Lys447) of the Fc region may or may not be present. Typically, an lgG Fc region comprises IgG CH2 and IgG CH3 domains, and optionally, on this basis, the lgG Fc region can also comprise a complete or partial hinge region, but does not comprise a CH1 domain. The “CH2 domain” of a human IgG Fc region generally extends from an amino acid residue at about position 231 to an amino acid residue at about position 340. In one embodiment, a carbohydrate chain is attached to the CH2 domain. The CH2 domain herein can be a native sequence CH2 domain or a variant CH2 domain. The “CH3 domain” comprises the stretch of residues at the C-terminus of the CH2 domain in the Fc region (i.e., from an amino acid residue at about position 341 to an amino acid residue at about position 447 of lgG). The CH3 region herein can be a native sequence CH3 domain or a variant CH3 domain (e.g., a CH3 domain having a “knob” introduced in one chain thereof and a “hole” correspondingly introduced in the other chain thereof; see U.S. Pat. No. 5,821,333, which is expressly incorporated herein by reference). As described herein, such variant CH3 domains can be used to facilitate heterodimerization of two non-identical antibody heavy chains.

Unless otherwise specified herein, the numbering of amino acid residues in an Fc region or a constant region is according to the EU numbering system, also known as the EU index, as described in Kabat et al., Sequences of Proteins of Immunological Interest, 5th Ed. Public Health Service, National Institutes of Health, Bethesda, MD, 1991.

The term “Fc variant” herein refers to changes in the structure or function of Fc caused by one or more amino acid substitution, insertion or deletion mutations at appropriate positions on the Fc. The “interaction between Fc variants” refers to the space-filling effect, electrostatic steering, hydrogen bonding effect, hydrophobic effect, etc. formed between Fc variants that have been subjected to a mutation design. The interaction between Fc variants contributes to the formation of a stable heterodimeric protein. A preferred mutation design is a mutation design in a “Knob-into-Hole” form.

The mutation design technique of Fc variants has been widely used in the field to prepare bispecific antibodies or heterodimeric Fc fusion protein forms. Representative ones include the “Knob-into-Hole” form proposed by Cater et al. (Protein Engineering vol. 9 no. 7 pp. 617-621, 1996); an Fc-containing heterodimer form formed by using Electrostatic Steering by the technician in Amgen (US20100286374 A1); The heterodimer form (SEEDbodies) formed by lgG/lg chain exchange proposed by Jonathan H. Davis et al. (Protein Engineering, Design & Selection pp. 1-8, 2010); Bispecific molecules formed by Genmab's DuoBody (Science, 2007.317 (5844)) platform technology; heterodimeric protein forms formed by combining structural calculation and Fc amino acid mutations and combining different modes of action by Xencor's technical staff (mAbs 3:6, 546-557; November/December 2011); a heterodimeric protein form obtained by the charge network-based Fc modification method of Alphamab Oncology (CN 201110459100.7); and other genetic engineering methods for forming heterodimeric functional proteins based on Fc amino acid changes or functional modification means. The Knob/Hole structure on the Fc variant fragments of the present application means that the two Fc fragments are mutated respectively, and can be combined in a “Knob-into-hole” form after the mutations. Preferably, the Fc regions are subjected to site mutation modification using the “Knob-into-hole” model of Cater et al., so that the obtained first Fc variant and second Fc variant can be combined together in a “Knob-into-hole” form to form a heterodimer. The selection of particular immunoglobulin Fc regions from particular immunoglobulin classes and subclasses is within the range of those skilled in the art. The Fc regions of human antibodies lgG1, IgG2, lgG3 and IgG4 are preferred, and the Fc region of human antibody IgG1 is more preferred. Either of the first Fc variant or the second Fc variant is randomly selected for knob mutation and the other for hole mutation.

The term “conservative amino acid” herein generally refers to amino acids that belong to the same class or have similar characteristics (such as charge, side chain size, hydrophobicity, hydrophilicity, backbone conformation and rigidity). Exemplarily, the following amino acids in each group are each other's conservative amino acid residues, and a substitution of amino acid residues in the group is a substitution of conservative amino acids:
Alanine (A), Serine(S) and Threonine (T);
Aspartic acid (D) and Glutamic acid (E);
Asparagine (N) and Glutamine (Q);
Arginine (R), Lysine (K) and Histidine (H);
Isoleucine (I), Leucine (L), Methionine (M) and Valine (V); and
Phenylalanine (F), Tyrosine (Y) and Tryptophan (W).

The term “identity” herein can be calculated by the following method: To determine the percent “identity” of two amino acid sequences or two nucleic acid sequences, the sequences are aligned for optimal comparison purposes (for example, gaps can be introduced in either or both of the first and second amino acid sequences or nucleic acid sequences for optimal alignment or non-homologous sequences can be discarded for comparison purposes). The amino acid residues or nucleotides at corresponding amino acid positions or nucleotide positions are then compared. When the position in the first sequence is occupied by the same amino acid residue or nucleotide as that at the corresponding position in the second sequence, the molecules are identical at that position. The percent identity between two sequences will vary with the identical positions shared by the sequences, considering the number of gaps that need to be introduced to optimally align the two sequences and the length of each gap.

The sequence comparison and the calculation of percent identity between two sequences can be achieved using a mathematical algorithm. For example, the percent identity between two amino acid sequences is determined using the Needleman and Wunsch algorithm ((1970) J. Mol. Biol. 48:444-453) in the GAP program that has been integrated into the GCG software package (available at www.gcg.com) and using the Blossum 62 matrix or the PAM250 matrix, with a gap weight of 16, 14, 12, 10, 8, 6 or 4 and a length weight of 1, 2, 3, 4, 5 or 6. For another example, the percent identity between two nucleotide sequences is determined using the GAP program in the GCG software package (available at www.gcg.com) and using the NWSgapdna.CMP matrix, with a gap weight of 40, 50, 60, 70, or 80 and a length weight of 1, 2, 3, 4, 5, or 6. A particularly preferred parameter set (and one parameter set that should be used unless otherwise stated) is the Blossum62 scoring matrix with a gap penalty of 12, a gap extension penalty of 4, and a gap frameshift penalty of 5. The percent identity between two amino acid sequences or nucleotide sequences can also be determined using the PAM120 weighted remainder table, with a gap length penalty of 12 and a gap penalty of 4, and using the E. Meyers and W. Miller algorithm ((1989) CABIOS, 4:11-17) that has been incorporated into the ALIGN program (version 2.0).

Additionally or alternatively, the nucleic acid and protein sequences of the present application can further be used as “query sequences” to perform searches against public databases, e.g., to identify other family member sequences or related sequences. For example, the NBLAST and XBLAST programs (version 2.0) of Altschul et al., (1990) J. Mol. Biol. 215:403-10 can be used to perform such searches. BLAST nucleotide searches can be performed with the NBLAST program, with score=100 and word length=12, to obtain the nucleotide sequences homologous to the nucleic acid molecules of the present application. BLAST protein searches can be performed with the XBLAST program, with score=50 and word length=3, to obtain the amino acid sequences homologous to the protein molecule of the present application. To obtain gapped alignment results for comparison purposes, gapped BLAST can be used as described in Altschul et al., (1997) Nucleic Acids Res. 25:3389-3402. When BLAST and gapped BLAST programs are used, the default parameters of the corresponding programs (e.g., XBLAST and NBLAST) can be used. See www.ncbi.nlm.nih.gov.

The term “chimeric antigen receptor (CAR)” herein refers to an artificial cell surface receptor engineered to express on immune effector cells and specifically bind to antigens, comprising at least (1) an extracellular antigen-binding domain, such as a heavy chain variable region and/or a light chain variable region of an antibody, (2) a transmembrane domain anchoring the CAR into an immune effector cell, and (3) an intracellular signaling domain. The CAR can redirect T cells and other immune effector cells to selected targets, such as cancer cells, in a non-MHC-restricted way by using an extracellular antigen-binding domain.

The term “nucleic acid” herein includes any compound and/or substance comprising a polymer of nucleotides. Each nucleotide consists of a base, specifically a purine or pyrimidine base (i.e., cytosine (C), guanine (G), adenine (A), thymine (T) or uracil (U)), a sugar (i.e., deoxyribose or ribose) and a phosphate group. Generally, a nucleic acid molecule is described by a sequence of bases, whereby the bases represent the primary structure (linear structure) of the nucleic acid molecule. The sequence of bases is usually expressed as 5′ to 3′. In this context, the term nucleic acid molecule encompasses deoxyribonucleic acid (DNA), including for example complementary DNA (cDNA) and genomic DNA, ribonucleic acid (RNA), especially messenger RNA (mRNA), synthetic forms of DNA or RNA, and a polymer comprising a mixture of two or more of these molecules. The nucleic acid molecule can be linear or circular. Furthermore, the term nucleic acid molecule includes both sense strand and antisense strand, as well as single-stranded form and double-stranded form. Furthermore, the nucleic acid molecules described herein may contain naturally occurring or non-naturally occurring nucleotides. Examples of non-naturally occurring nucleotides include modified nucleotide bases with derivatized sugar or phosphate backbone linkages or chemically modified residues. The nucleic acid molecule also encompasses DNA and RNA molecules which are suitable as vectors for direct expression of the antibody of the present application in vitro and/or in vivo, for example in a host or patient. Such DNA (e.g., cDNA) or RNA (e.g., mRNA) vectors may be unmodified or modified. For example, mRNA can be chemically modified to enhance the stability of the RNA vector and/or the expression of the encoded molecule, so that the mRNA can be injected into a subject to generate an antibody in vivo (see, for example, Stadler et al., Nature Medicine 2017, Published online Jun. 12, 2017, doi: 10.1038/nm.4356 or EP 2101823 B1).

The “isolated” nucleic acid herein refers to a nucleic acid molecule that has been separated from components of the natural environment thereof. The isolated nucleic acid includes a nucleic acid molecule contained in a cell that normally contains the nucleic acid molecule, but which is present extrachromosomally or at a chromosomal location other than the natural chromosomal location thereof.

The term “vector” herein refers to a nucleic acid molecule capable of amplifying another nucleic acid to which it is linked. The term includes a vector having a self-replicating nucleic acid structure and a vector that is integrated into the genome of a host cell into which the vector has been introduced. Certain vectors can direct the expression of the nucleic acid to which they are operably linked. Such vectors are referred to herein as “expression vectors”.

The term “host cell” herein refers to a cell into which a foreign nucleic acid is introduced, including the progenies of such a cell. The host cell includes “transformant” and “transformed cell” which include the primary transformed cell and progeny derived therefrom, regardless of the number of passages. The progeny may not be identical to the parental cell in nucleic acid content, and may contain a mutation. The mutant progeny with the same function or biological activity as those screened or selected in the initially transformed cells are included herein.

As used herein, the term “pharmaceutical composition” refers to a preparation that is present in a form which allows the active ingredients contained therein to be biologically effective and does not contain additional ingredients that would be unacceptably toxic to the subject to which the pharmaceutical composition is administered.

The term “pharmaceutically acceptable carrier” herein includes any and all solvents, dispersion media, coating materials, surfactants, antioxidants, preservatives (e.g., antibacterial agents and antifungal agents), isotonic agents, absorption delaying agents, salts, preservatives, drug stabilizers, binders, excipients, disintegrants, lubricants, sweeteners, flavoring agents, dyes, etc., and a combination thereof, which are known to those skilled in the art (see, for example, Remington's Pharmaceutical Sciences, 18th edition. MackPrinting Company, 1990, pages 1289-1329). Except in cases of incompatibility with the active ingredient, any conventional carrier is contemplated for use in a therapeutic or pharmaceutical composition.

The term “treatment” herein refers to surgical or therapeutic treatment, the purpose of which is to prevent and slow down (reduce) an undesired physiological change or pathology in a subject being treated, such as cancers and tumors. Beneficial or desired clinical outcomes include, but are not limited to, the alleviation of symptoms, the weakening of disease severity, the stabilization of disease state (i.e., no deterioration), the delay or slowing down of disease progression, the amelioration or mitigation of disease state, and remission (whether partial or complete), whether detectable or undetectable. Objects in need of treatment include those who already have a disorder or disease, those who are susceptible to a disorder or disease or those who intend to prevent a disorder or disease. When terms such as slowing down, alleviation, weakening, mitigation, and remission are referred to, their meanings also include elimination, disappearance, non-occurrence and other circumstances.

The term “subject” refers to an organism receiving treatment for a particular disease or condition as described herein. Exemplarily, a “subject” includes a mammal, such as a human, a primate (e.g., monkey) or a non-primate mammal, receiving treatment for a disease or a condition.

The term “effective amount” herein refers to an amount of a therapeutic agent effective to prevent or relieve a disease or a disorder or the progression of the disease when administered alone or in combination with another therapeutic agent to a cell, tissue or subject. The “effective amount” also refers to an amount of a compound sufficient to relieve symptoms, such as treating, curing, preventing or relieving related medical disorders, or increasing the speed of treating, curing, preventing or relieving these disorders. When the active ingredient is administered to an individual alone, a therapeutically effective dose refers to the ingredient alone. When a certain combination is applied, a therapeutically effective dose refers to the combined dosage of the active ingredients that produce a therapeutic effect, whether administered in combination, sequentially or simultaneously.

The term “cancer” herein refers to or describes the physiological condition in mammals that is typically characterized by unregulated cell growth. Both benign and malignant cancers are included in this definition. The term “tumor” or “neoplasm” herein refers to all neoplastic cell growth and proliferation, whether malignant or benign, and to all pre-cancerous and cancerous cells and tissues. The terms “cancer” and “tumor” are not mutually exclusive when referred to herein.

The term “EC50” herein refers to the half-maximal effective concentration, which includes the concentration of an antibody that induces a response halfway between baseline and maximum after a specified exposure time. EC50 essentially represents the concentration of an antibody at which 50% of the maximal effect thereof is observed and which can be measured by methods known in the art.

### Detailed Description of Embodiments

The present application discloses an antibody or an antigen-binding fragment thereof that specifically binds to B cell mature antigen (BCMA), a multispecific antigen-binding molecule, a nucleic acid fragment, a vector, a host cell, an immune effector cell, a preparation method, a pharmaceutical composition, a pharmaceutical use, and a method of treating a tumor or a cancer (e.g., B cell lymphoma or multiple myeloma). The antibody can block the binding of BCMA's natural ligands (such as BAFF, APRIL) to BCMA.

In a first aspect, the present application provides an antibody or an antigen-binding fragment thereof that specifically binds to B cell maturation antigen (BCMA), wherein the antibody or the antigen-binding fragment thereof comprises a light chain variable region (VL) and a heavy chain variable region (VH), and wherein
(1) the light chain variable region comprises LCDR1, LCDR2 and LCDR3, the LCDR1 has any of a sequence of the following LCDR1, or a sequence with 1, 2, 3 or more amino acid insertions, deletions and/or substitutions compared to the sequence, the LCDR2 has any of a sequence of the following LCDR2, or a sequence with 1, 2, 3 or more amino acid insertions, deletions and/or substitutions compared to the sequence, and the LCDR3 has any of a sequence of the following LCDR3, or a sequence with 1, 2, 3 or more amino acid insertions, deletions and/or substitutions compared to the sequence:

| No. | LCDR1 | LCDR2 | LCDR3 |
|---|---|---|---|
| VL1 | SEQ ID NO: 107 | SEQ ID NO: 108 | SEQ ID NO: 109 |
| VL2 | SEQ ID NO: 110 | SEQ ID NO: 111 | SEQ ID NO: 109 |
| VL3 | SEQ ID NO: 118 | SEQ ID NO: 119 | SEQ ID NO: 120 |
| VL4 | SEQ ID NO: 121 | SEQ ID NO: 122 | SEQ ID NO: 120 |
| VL5 | SEQ ID NO: 129 | SEQ ID NO: 130 | SEQ ID NO: 131 |
| VL6 | SEQ ID NO: 132 | SEQ ID NO: 133 | SEQ ID NO: 131 |
| VL7 | SEQ ID NO: 140 | SEQ ID NO: 141 | SEQ ID NO: 142 |
| VL8 | SEQ ID NO: 143 | SEQ ID NO: 144 | SEQ ID NO: 142 |
| VL9 | SEQ ID NO: 151 | SEQ ID NO: 152 | SEQ ID NO: 153 |
| VL10 | SEQ ID NO: 154 | SEQ ID NO: 155 | SEQ ID NO: 153 |
| VL11 | SEQ ID NO: 162 | SEQ ID NO: 163 | SEQ ID NO: 164 |
| VL12 | SEQ ID NO: 165 | SEQ ID NO: 166 | SEQ ID NO: 164 |
| VL13 | SEQ ID NO: 173 | SEQ ID NO: 174 | SEQ ID NO: 175 |
| VL14 | SEQ ID NO: 176 | SEQ ID NO: 177 | SEQ ID NO: 175 |
| VL15 | SEQ ID NO: 178 | SEQ ID NO: 108 | SEQ ID NO: 109 |
| VL16 | SEQ ID NO: 162 | SEQ ID NO: 163 | SEQ ID NO: 183 |
| VL17 | SEQ ID NO: 162 | SEQ ID NO: 163 | SEQ ID NO: 184 |

and,
(2) the heavy chain variable region comprises HCDR1, HCDR2 and HCDR3, the HCDR1 has any of a sequence of the following HCDR1, or a sequence with 1, 2, 3 or more amino acid insertions, deletions and/or substitutions compared to the sequence, the HCDR2 has any of a sequence of the following HCDR2, or a sequence with 1, 2, 3 or more amino acid insertions, deletions and/or substitutions compared to the sequence, and the HCDR3 has any of a sequence of the following HCDR3, or a sequence with 1, 2, 3 or more amino acid insertions, deletions and/or substitutions compared to the sequence:

| No. | HCDR1 | HCDR2 | HCDR3 |
|---|---|---|---|
| VH1 | SEQ ID NO: 101 | SEQ ID NO: 102 | SEQ ID NO: 103 |
| VH2 | SEQ ID NO: 104 | SEQ ID NO: 105 | SEQ ID NO: 106 |
| VH3 | SEQ ID NO: 112 | SEQ ID NO: 113 | SEQ ID NO: 114 |
| VH4 | SEQ ID NO: 115 | SEQ ID NO: 116 | SEQ ID NO: 117 |
| VH5 | SEQ ID NO: 123 | SEQ ID NO: 124 | SEQ ID NO: 125 |
| VH6 | SEQ ID NO: 126 | SEQ ID NO: 127 | SEQ ID NO: 128 |
| VH7 | SEQ ID NO: 134 | SEQ ID NO: 135 | SEQ ID NO: 136 |
| VH8 | SEQ ID NO: 137 | SEQ ID NO: 138 | SEQ ID NO: 139 |
| VH9 | SEQ ID NO: 145 | SEQ ID NO: 146 | SEQ ID NO: 147 |
| VH10 | SEQ ID NO: 148 | SEQ ID NO: 149 | SEQ ID NO: 150 |
| VH11 | SEQ ID NO: 156 | SEQ ID NO: 157 | SEQ ID NO: 158 |
| VH12 | SEQ ID NO: 159 | SEQ ID NO: 160 | SEQ ID NO: 161 |
| VH13 | SEQ ID NO: 167 | SEQ ID NO: 168 | SEQ ID NO: 169 |
| VH14 | SEQ ID NO: 170 | SEQ ID NO: 171 | SEQ ID NO: 172 |
| VH15 | SEQ ID NO: 101 | SEQ ID NO: 179 | SEQ ID NO: 103 |
| VH16 | SEQ ID NO: 123 | SEQ ID NO: 180 | SEQ ID NO: 125 |
| VH17 | SEQ ID NO: 134 | SEQ ID NO: 181 | SEQ ID NO: 136 |
| VH18 | SEQ ID NO: 145 | SEQ ID NO: 182 | SEQ ID NO: 147 |

In a preferred embodiment, the antibody or the antigen-binding fragment thereof comprises the sequences of six CDRs in the following combination of light chain variable region and heavy chain variable region: VL1+VH1, VL2+VH2, VL3+VH3, VL4+VH4, VL5+VH5, VL6+VH6, VL7+VH7, VL8+VH8, VL9+VH9, VL10+VH10, VL11+VH11, VL12+VH12, VL13+VH13, VL14+VH14, VL15+VH1, VL1+VH15, VL15+VH15, VL5+VH16, VL7+VH17, VL9+VH18, VL16+VH11 or VL17+VH11, or sequences of six CDRs having 1, 2, 3 or more amino acid insertions, deletions and/or substitutions compared to the sequences of the six CDRs.

In a particular embodiment, the present application provides an antibody or an antigen-binding fragment thereof, wherein:
(1) the light chain variable region sequence comprises a sequence as shown in any one of SEQ ID NO: 8, 10, 12, 14, 16, 18, 20, 21-23, 32-33, 41-43, 52-53, 65-67, 78-81 and 90-93, or a sequence having 80%, 85%, 90%, 95%, 96%, 97%, 98%, 99% or higher identity to the sequence;
   and,
(2) the heavy chain variable region sequence comprises a sequence as shown in any one of SEQ ID NO: 7, 9, 11, 13, 15, 17, 19, 24-27, 34-35, 44-47, 54-59, 68-72, 82-84 and 94-96, or a sequence having 80%, 85%, 90%, 95%, 96%, 97%, 98%, 99% or higher identity to the sequence.

In a preferred embodiment, the antibody or the antigen-binding fragment thereof has a light chain variable region and a heavy chain variable region as follows:
(1) the light chain variable region and the heavy chain variable region comprise sequences as shown in SEQ ID NO: 8 and SEQ ID NO: 7, respectively;
(2) the light chain variable region and the heavy chain variable region comprise sequences as shown in SEQ ID NO: 10 and SEQ ID NO: 9, respectively;
(3) the light chain variable region and the heavy chain variable region comprise sequences as shown in SEQ ID NO: 12 and SEQ ID NO: 11, respectively;
(4) the light chain variable region and the heavy chain variable region comprise sequences as shown in SEQ ID NO: 14 and SEQ ID NO: 13, respectively;
(5) the light chain variable region and the heavy chain variable region comprise sequences as shown in SEQ ID NO: 16 and SEQ ID NO: 15, respectively;
(6) the light chain variable region and the heavy chain variable region comprise sequences as shown in SEQ ID NO: 18 and SEQ ID NO: 17, respectively;
(7) the light chain variable region and the heavy chain variable region comprise sequences as shown in SEQ ID NO: 20 and SEQ ID NO: 19, respectively;
(8) the light chain variable region comprises a sequence as shown in any one of SEQ ID NOs: 21-23, and the heavy chain variable region comprises a sequence as shown in any one of SEQ ID NOs: 24-27;
(9) the light chain variable region comprises a sequence as shown in any one of SEQ ID NOs: 32-33, and the heavy chain variable region comprises a sequence as shown in any one of SEQ ID NOs: 34-35;
(10) the light chain variable region comprises a sequence as shown in any one of SEQ ID NOs: 41-43, and the heavy chain variable region comprises a sequence as shown in any one of SEQ ID NOs: 44-47;
(11) the light chain variable region comprises a sequence as shown in any one of SEQ ID NOs: 52-53, and the heavy chain variable region comprises a sequence as shown in any one of SEQ ID NOs: 54-59;
(12) the light chain variable region comprises a sequence as shown in any one of SEQ ID NOs: 65-67, and the heavy chain variable region comprises a sequence as shown in any one of SEQ ID NOs: 68-72;
(13) the light chain variable region comprises a sequence as shown in any one of SEQ ID NOs: 78-81, and the heavy chain variable region comprises a sequence as shown in any one of SEQ ID NOs: 82-84;
(14) the light chain variable region comprises a sequence as shown in any one of SEQ ID NOs: 90-93, and the heavy chain variable region comprises a sequence as shown in any one of SEQ ID NOs: 94-96; or
(15) the light chain variable region comprises a sequence having 80%, 85%, 90%, 95%, 96%, 97%, 98%, 99% or higher identity to the light chain variable region as shown in any one of the above (1) to (14), and the heavy chain variable region comprises a sequence having 80%, 85%, 90%, 95%, 96%, 97%, 98%, 99% or higher identity to the heavy chain variable region as shown in any one of the above (1) to (14).

In a particular embodiment, the antibody or the antigen-binding fragment thereof is chimeric, humanized or fully human.

In a particular embodiment, the antibody or the antigen-binding fragment thereof is capable of binding to human or monkey BCMA.

In a specific embodiment, the antibody or the antigen-binding fragment thereof binds to human BCMA with a dissociation constant of no greater than 10⁻⁷ M or 10⁻⁸ M or 10⁻⁹ M.

In a specific embodiment, the antibody or the antigen-binding fragment thereof blocks the binding of APRIL to human BCMA.

In a particular embodiment, the antibody or the antigen-binding fragment thereof comprises a constant region sequence of any one of human or murine antibody IgG1, lgG2, lgG3, lgG4, IgA, IgM, IgE or IgD; preferably, the antibody or the antigen-binding fragment thereof comprises a constant region sequence of human or murine antibody lgG1, lgG2, lgG3 or lgG4, or comprises a sequence having 80%, 85%, 90%, 95%, 96%, 97%, 98%, 99% or higher identity with the constant region sequence of human or murine antibody IgG1, lgG2, lgG3 or lgG4.

In a particular embodiment, the antigen-binding fragment is selected from one or more of F(ab′) ₂, Fab′, Fab, Fv, scFv, nanobody or affibody.

In a second aspect, the present application further provides a multispecific antigen-binding molecule, wherein the multispecific antigen-binding molecule comprises the aforementioned antibody or the antigen-binding fragment thereof, and an antigen-binding molecule binding to an additional antigen other than BCMA or binding to a different BCMA epitope from that of the aforementioned antibody or the antigen-binding fragment thereof; optionally, the additional antigen other than BCMA is selected from: CD3 (preferably CD3ε), CD16, CD137, CD258, PD-1, PD-L1, 4-1BB, CD40, CD64, EGFR, VEGF, HER2, HER1, HER3, IGF-1R, phosphatidylserine (PS), C-Met, HSA, GPRC5D, MSLN, blood-brain barrier receptor, GPC3, PSMA, CD33, GD2, ROR1, ROR2, FRα or Gucy2C.

Preferably, the additional antigen-binding molecule is an antibody or an antigen-binding fragment thereof.

Preferably, the multispecific antigen-binding molecule can be bispecific, trispecific or tetraspecific.

Preferably, the multispecific antigen-binding molecule can be bivalent, trivalent, tetravalent, pentavalent or hexavalent.

In a third aspect, the present application further provides a chimeric antigen receptor (CAR), wherein the chimeric antigen receptor at least comprises a signal peptide, an extracellular antigen-binding domain, a hinge region, a transmembrane domain, and an intracellular signaling domain, the extracellular antigen-binding domain comprises the BCMA antibody or the antigen-binding fragment thereof of any one of the aforementioned aspects, or the aforementioned multispecific antigen-binding molecule.

In a fourth aspect, the present application further provides an immune effector cell, wherein the immune effector cell expresses the aforementioned chimeric antigen receptor, or contains a nucleic acid fragment encoding the aforementioned chimeric antigen receptor; preferably, the immune effector cell is selected from a T cell, a natural killer cell (a NK cell), an NKT cell (a natural killer T cell), a DNT cell (a double negative T cell), a monocyte, a macrophage, a dendritic cell or a mast cell, and the T cell is preferably selected from a cytotoxic T cell (CTL), a regulatory T cell or a helper T cell; and preferably, the immune effector cell is an autologous immune effector cell or an allogeneic immune effector cell.

In a fifth aspect, the present application further provides an isolated nucleic acid fragment, wherein the nucleic acid fragment encodes the aforementioned antibody or the antigen-binding fragment thereof, the multispecific antigen-binding molecule, or the chimeric antigen receptor.

In a sixth aspect, the present application further provides a vector, wherein the vector comprises the aforementioned nucleic acid fragment.

In a seventh aspect, the present application further provides a host cell, wherein the host cell comprises the aforementioned vector; preferably, the cell is a prokaryotic cell or a eukaryotic cell, such as a bacterial (for example, Escherichia coli) cell, a fungal (for example, yeast) cell, an insect cell or a mammalian cell (for example, a CHO cell line or a 293T cell line).

In an eighth aspect, the present application further provides a method of preparing the antibody or the antigen-binding fragment thereof or the aforementioned multispecific antigen-binding molecule, wherein the method comprises culturing the aforementioned cell, and isolating the antibody, antigen-binding fragment or multispecific antigen-binding molecule expressed by the cell.

In a ninth aspect, the present application further provides a method of preparing the aforementioned immune effector cell, comprising introducing a nucleic acid fragment encoding the aforementioned CAR into the immune effector cell, and optionally, further comprising enabling the immune effector cell to express the aforementioned CAR.

In a tenth aspect, the present application further provides a pharmaceutical composition, wherein the pharmaceutical composition comprises the aforementioned antibody or the antigen-binding fragment thereof, the multispecific antigen-binding molecule, the immune effector cell, the nucleic acid fragment or the vector, or a product prepared according to the aforementioned method; optionally, the pharmaceutical composition also comprises a pharmaceutically acceptable carrier, a diluent or an auxiliary agent; and optionally, the pharmaceutical composition further comprises an additional anti-tumor agent.

In some embodiments, the pharmaceutically acceptable carrier is a carrier that does not weaken the viability and function of immune cells and does not affect the specific binding of antibodies or antigen-binding fragments thereof to antigens, and includes but not limited to cell culture media, buffers, physiological saline, balanced salt solution, etc. Examples of buffers include isotonic phosphate, acetate, citrate, borate, carbonate, etc. In a particular embodiment, the pharmaceutically acceptable carrier is phosphate buffered saline containing 1% serum.

In an eleventh aspect, the present application further provides a method of treating a tumor or a cancer, wherein the method comprises administering to a subject an effective amount of the aforementioned antibody or antigen-binding fragment thereof, the multispecific antigen-binding molecule, the immune effector cell, nucleic acid fragment, the vector, a product prepared according to the aforementioned method or the pharmaceutical composition; the tumor or the cancer is a BCMA-expressing tumor or cancer, preferably, B-cell lymphoma; more preferably multiple myeloma (MM).

In a twelfth aspect, the present application further provides the use of the aforementioned antibody or the antigen-binding fragment thereof, the multispecific antigen-binding molecule, the immune effector cell, the nucleic acid fragment, the vector, a product prepared according to the aforementioned method or the pharmaceutical composition for the manufacture of a medicament for the treatment of a tumor or a cancer; the tumor or the cancer is a BCMA-expressing tumor or cancer, preferably, B-cell lymphoma; more preferably multiple myeloma (MM).

In a thirteenth aspect, the present application further provides the aforementioned antibody or the antigen-binding fragment thereof, the multispecific antigen-binding molecule, the immune effector cell, the nucleic acid fragment, the vector, a product prepared according to the aforementioned method or the pharmaceutical composition for use in the treatment of a tumor or a cancer; the tumor or the cancer is a BCMA-expressing tumor or cancer, preferably, B-cell lymphoma; more preferably multiple myeloma (MM).

In a fourteenth aspect, the present application further provides a kit, wherein the kit comprises the aforementioned antibody or the antigen-binding fragment thereof, the multispecific antigen-binding molecule, the immune effector cell, the nucleic acid fragment, the vector, a product prepared according to the aforementioned method or the pharmaceutical composition.

In a fifteenth aspect, the present application further provides a method of detecting BCMA expression in a biological sample, wherein the method comprises contacting the biological sample with the aforementioned antibody or the antigen-binding fragment thereof under conditions that allow the formation of a complex from the antibody or the antigen-binding fragment thereof and BCMA; and preferably, the method further comprises detecting the formation of the complex, thereby indicating the presence or expression level of BCMA in the sample.

In a sixteenth aspect, the present application further provides the use of the aforementioned antibody or the antigen-binding fragment thereof in preparing a BCMA detection reagent.

### EXAMPLES

The present application will be further described below in conjunction with specific examples, and the advantages and characteristics of the present application will become clearer along with the description. If no specific conditions are indicated in the examples, conventional conditions or the conditions suggested by the manufacturer shall be followed. Any reagents or instruments used, unless the manufactures stated, are conventional products that are commercially available.

The examples of the present application are only exemplary and do not limit the scope of the present application in any way. Those skilled in the art should understand that the details and forms of the technical solutions of the present application can be modified or replaced without departing from the spirit and scope of the present application, but these modifications and replacements all fall within the scope of protection of the present application.

### Example 1 Preparation of Control Antibody, Identification of Endogenous Cell and Preparation of Overexpression Cell Strain

### (a) Preparation of Control Antibodies

REGN5459 and HPN217 are both antibodies that recognize human BCMA protein. The sequence of REGN5459 comes from US Patent Publication No. US 2020/0024356 A1, and the HPN217 sequence comes from US Patent Publication No. US20190112381 A1. The heavy chain variable region (VH) of REGN5459 was recombined into an expression vector containing a signal peptide and human antibody IgG1 heavy chain constant region, and the light chain variable region (VL) sequence was recombined into an expression vector containing a signal peptide and human antibody IgG1 light chain, thus a recombinant plasmid was obtained and the synthesized antibody was named REGN5459-hlgG1. The VHH sequence of HPN217 was recombined into an expression vector containing a signal peptide and human antibody IgG1 Fc to obtain a recombinant plasmid. The synthesized antibody was named HPN217-hHcAb, and had a sequence as shown in Table 1. The negative control antibody hlgG1 was the antibody anti-hel-hlgG1 (purchased from Biointron, catalog number: B117901, hereinafter referred to as hlgG1) against Hen Egg Lysozyme chicken egg lysozyme.

TABLE 1
List of sequences of control antibodies

| Designation of | Sequence | |
|---|---|---|
| sequence | number | Amino acid sequence |
| REGN5459 VH | SEQ ID | EVQLVESGGGLVQPGGSLRLSCAASGFTFSNFWMTWV |
| | NO: 1 | RQAPGKGLEWVANMNQDGSEKYYVDSVKGRFTISRDN |
| | | AKSSLYLQMNSLRAEDTAVYYCARDREYCISTSCYDD |
| | | FDYWGQGTLVTVSS |
| REGN5459 VL | SEQ ID | DIQMTQSPSSLSASVGDRVTITCRASQSISSYLNWYQQK |
| | NO: 2 | PGKAPKLLIYAASSLQSGVPSRFSGSGSGTDFTLTISSL |
| | | QPEDFATYYCQQSYSTPPITFGQGTRLEIK |
| HPN217 VHH | SEQ ID | EVQLVESGGGLVQPGRSLTLSCAASTNIFSISPYGWYRQ |
| | NO: 3 | APGKQRELVAAIHGTSTLYADSVKGRFTISRDNAKNSIY |
| | | LQMNSLRPEDTALYYCNKVPWGDYHPGNVYWGQGTQVTV |
| | | SS |
| Human IgG1 heavy | SEQ ID | ASTKGPSVFPLAPSSKSTSGGTAALGCLVKDYFPEPVTV |
| chain constant | NO: 4 | SWNSGALTSGVHTFPAVLQSSGLYSLSSVVTVPSSSLGT |
| region | | QTYICNVNHKPSNTKVDKKVEPKSCDKTHTCPPCPAPEL |
| | | LGGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALPAPIEKTISKAKGQPREPQVYT |
| | | LPPSREEMTKNQVSLTCLVKGFYPSDIAVEWESNGQPE |
| | | NNYKTTPPVLDSDGSFFLYSKLTVDKSRWQQGNVFSCS |
| | | VMHEALHNHYTQKSLSLSPGK |
| Human IgG1 light | SEQ ID | RTVAAPSVFIFPPSDEQLKSGTASVVCLLNNFYPREAKV |
| chain constant | NO: 5 | QWKVDNALQSGNSQESVTEQDSKDSTYSLSSTLTLSKA |
| region | | DYEKHKVYACEVTHQGLSSPVTKSFNRGEC |
| Human IgG1 Fc | SEQ ID | EPKSSDKTHTCPPCPAPELLGGPSVFLFPPKPKDTLMIS |
| | NO: 6 | RTPEVTCVVVDVSHEDPEVKFNWYVDGVEVHNAKTKPR |
| | | EEQYNSTYRVVSVLTVLHQDWLNGKEYKCKVSNKALPA |
| | | PIEKTISKAKGQPREPQVYTLPPSREEMTKNQVSLTCLVK |
| | | GFYPSDIAVEWESNGQPENNYKTTPPVLDSDGSFFLYSK |
| | | LTVDKSRWQQGNVFSCSVMHEALHNHYTQKSLSLSPG |
| | | K |

Notes:
The human IgG1 Fc sequence contains the C220S mutation.

The binding activity of the control antibody to human BCMA-His protein (purchased from Acro, catalog number: BCA-H522y) and monkey BCMA-His protein (purchased from Acro, catalog number: BCA-C52H7) was detected by ELISA. The specific method was as follows: The antigen protein was diluted with PBS to a final concentration of 1 μg/mL, and then added to a 96-well ELISA plate at 50 μl per well. The plate was sealed with a plastic film and incubated overnight at 4° C., the plate was washed twice with PBS the next day, and then a blocking solution [PBS+2% (w/v) BSA] was added for blocking at room temperature for 2 h. The blocking solution was poured off and 50 μl of 100 nM serially diluted control antibody or negative control antibody was added to each well. After incubation at 37° C. for 2 h, the plate was washed 3 times with PBS. HRP (horseradish peroxidase)-labeled secondary antibody (purchased from Merck, catalog number: AP113P) was added, and incubated at 37° C. for 1 h, and the plate was washed 5 times with PBS. TMB substrate was added at 50 μl/well, and incubated at room temperature for 10 min, then a stop solution (1.0 M HCl) was added at 50 μl/well. The OD450 nm values were read by an ELISA microplate reader (Multimode Plate Reader, EnSight, purchased from Perkin Elmer). The results are shown in Tables 2 and 3 and FIGS. 1A and 1B. REGN5459-hlgG1 antibody has good binding activity with both human BCMA protein and monkey BCMA protein. HPN217-hHcAb has good binding activity with human BCMA protein, but does not bind to monkey BCMA protein, and the IgG subtype control is human IgG1.

TABLE 2
ELISA detection of binding activity of control
antibody with human BCMA-his protein

| | Antibody concentration (nM) | | | | | | | |
|---|---|---|---|---|---|---|---|---|
| Designation | | | | | | | | Blank |
| of antibody | 100 | 10 | 1 | 0.1 | 0.01 | 0.001 | 0.0001 | control |
| REGN5459- | 2.89 | 2.63 | 2.00 | 1.54 | 0.50 | 0.14 | 0.07 | 0.06 |
| hIgG1 | | | | | | | | |
| HPN217-hHcAb | 2.75 | 2.64 | 2.34 | 1.39 | 0.56 | 0.24 | 0.17 | 0.07 |
| hIgG1 | 0.12 | 0.07 | 0.05 | 0.05 | 0.05 | 0.05 | 0.05 | 0.06 |

TABLE 3
ELISA detection of binding activity of control
antibody with monkey BCMA-his protein

| | Antibody concentration (nM) | | | | | | | |
|---|---|---|---|---|---|---|---|---|
| Designation | | | | | | | | Blank |
| of antibody | 100 | 10 | 1 | 0.1 | 0.01 | 0.001 | 0.0001 | control |
| REGN5459- | 3.10 | 2.82 | 2.57 | 2.06 | 0.78 | 0.21 | 0.09 | 0.06 |
| hIgG1 | | | | | | | | |
| HPN217-hHcAb | 0.16 | 0.06 | 0.05 | 0.05 | 0.05 | 0.05 | 0.05 | 0.05 |
| hIgG1 | 0.10 | 0.06 | 0.05 | 0.05 | 0.04 | 0.05 | 0.05 | 0.05 |

### (B) Identification of Cell Strains Expressing BCMA Protein Endogenously

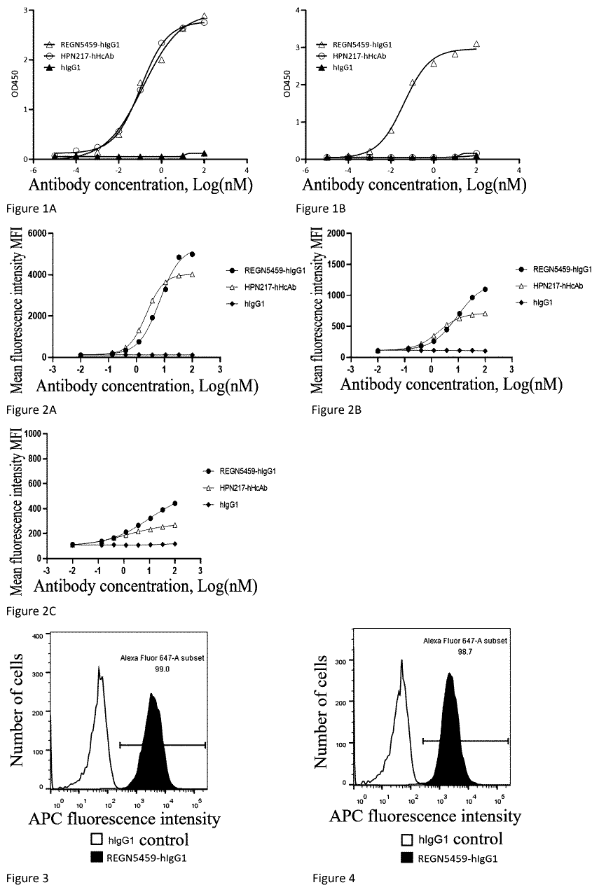

H929 cells, U266 cells and RPMI8226 cells were subjected to scale-up culture in T-75 cell culture flask to logarithmic growth phase, and the cells were pipetted to single cell suspension. After counting the cells, centrifugation was performed and the cell pellet was resuspended in FACS buffer (PBS+2% fetal calf serum) to 2×10⁶ cells per ml, and the obtained suspension was added to a 96-well FACS reaction plate at 50 μl per well. REGN5459-hlgG1 and HPN217-hHcAb antibodies were used as primary antibodies, and APC-labeled goat anti-human IgG (H+L) secondary antibody (purchased from Jackson, catalog number: 109-605-088) were detected and analyzed by FACS (FACS Canto™, purchased from BD Company). The results are shown in Table 4 and FIGS. 2A, 2B, and 2C, indicating that H929 cells, U266 cells, and RPMI8226 cells can bind to REGN5459-hlgG1 and HPN217-hHcAb antibodies.

TABLE 4
FACS detection results of endogenous cell lines H929, U266 and RPMI8226

| | H929 | | U266 | | RPMI8226 | |
|---|---|---|---|---|---|---|
| | Maximum | | Maximum | | Maximum | |
| | mean | | mean | | mean | |
| Designation | fluorescence | EC50 | fluorescence | EC50 | fluorescence | EC50 |
| of antibody | intensity | (nM) | intensity | (nM) | intensity | (nM) |
| REGN5459- | 4993 | 6.78 | 1097 | 9.12 | 443 | 10.38 |
| hIgG1 | | | | | | |
| HPN217- | 4015 | 2.24 | 708 | 1.83 | 269 | 1.38 |
| hHcAb | | | | | | |
| hIgG1 | 110 | Negative | 103 | Negative | 118 | Negative |

### (C) Preparation of Flp-inCHO Recombinant Cell Strains Expressing Human BCMA Protein and Monkey BCMA Protein Respectively

The nucleotide sequences encoding the amino acid sequences of human BCMA (NCBI Gene ID: 608) and monkey BCMA (NCBI Gene ID: 712212) were cloned into the pcDNA5/FRT vector (purchased from Clontech), respectively. The Flp-inCHO cell lines (purchased from the cell bank of the Type Culture Collection Committee of Chinese Academy of Sciences) were transfected (Lipofectamine® 3000 Transfection Kit, purchased from Invitrogen, catalog number: L3000-015) respectively, and then selectively cultured in F12K Medium (Gibco, catalog number: 21127030) containing 600 μg/ml hygromycin (Thermofisher, catalog number: 10687010) and 10% (v/v) fetal bovine serum (ExCell Bio, catalog number: FND500) for 2 weeks. REGN5459-hlgG1 and goat anti-human IgG (H+L) antibody (Jackson, catalog number: 109605088) were used for detection on FACS Cantoll (purchased from BD Biosciences), and the cells with high expression and single peak shape were proliferated, and the proliferated cells were retested by flow cytometry. The positive cell pool with better growth, higher fluorescence intensity and better homogeneity was selected for further scale-up culture and then cryopreserved in liquid nitrogen. Table 5 and FIGS. 3 and 4 show that a Flp-inCHO high-expression cell population with human BCMA-positive expression and a Flp-inCHO high-expression cell population with monkey BCMA-positive expression have been prepared respectively. In FIGS. 3 and 4, the abscissa was the fluorescence intensity of cells, and the ordinate was the number of cells.

TABLE 5
FACS detection results of Flp-inCHO recombinant cell
line expressing human and monkey BCMA protein

| | | Cell mean fluorescence density | |
|---|---|---|---|
| | Stably transfected | IgG subtype | BCMA |
| No. | positive cell pool | control | antibody |
| 1 | Flp-in CHO-human BCMA | 54.6 | 4313 |
| 2 | Flp-in CHO-monkey BCMA | 49 | 2984 |

### Example 2 Preparation of Hybridoma Nanobody Against BCMA

### (a) Immunization and Serum Titer Detection of Mouse

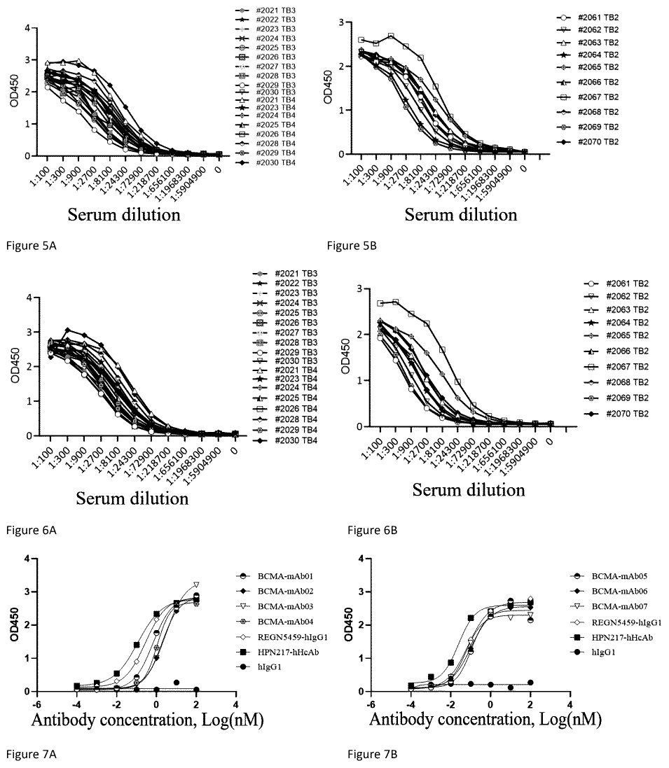

Female SJL mice aged 6-8 weeks (purchased from Shanghai Slack Experimental Animal Co., Ltd.) were divided into two groups: G1 and G2. In both group, human BCMA (Met 1-Ala 54)-hFc protein (purchased from Acro, catalog number: BC7-H5254), Titer max (purchased from sigma, catalog number: T2684) and oligonucleotide CPG (ODN 1826, synthesized by Sangon Biotech (Shanghai) Co., Ltd.) were mixed and emulsified and injected into the footpads and back; human BCMA-hFc protein, Imject Alum (purchased from Thermo fisher scientific, catalog number: 77161) and CPG were mixed and injected into the abdominal cavity; each mouse was injected with 50 μg of antigen. For the first booster immunization, cynomolgus monkey BCMA (Met 1-Ala 53)-hFc protein (purchased from Acro, catalog number: BCA-C5253), Imject Alum and CPG were mixed evenly, and then the mixture was injected into the back and footpads. For the second booster immunization, human BCMA-hFc protein, titer max and CPG were mixed and emulsified and then injected into the back. Subsequent booster immunizations were carried out in an alternating manner between the first and second booster immunizations, with 25 μg of antigen injected into each mouse each time, with an interval of 7 days between each immunization. Blood collection of mice in the G1 group was performed on the 5th day after the fifth (TB3) and sixth (TB4) booster immunization respectively. Blood collection of mice in the G2 group were performed on the 5th day after the fourth (TB2) boosted immunization. The serum was separated and the titers of specific antibodies in the serum were determined using the enzyme-linked immunosorbent (ELISA) method. The experimental results are shown in FIGS. 5A and 5B and FIGS. 6A and 6B, which show that after immunization, the serum of the immunized mice demonstrated high titers of binding to human and monkey BCMA, indicating that the sera of the immunized mice have varying degrees of binding to the immunogen, showing an antigen-antibody reaction. The ELISA blank control was 1% (w/v) BSA.

### (B) Splenocyte Fusion and Hybridoma Screening

A total of 50 μg of huBCMA-huFc immunogen was injected into the abdominal cavity, footpad and back of each selected mouse. The mice were sacrificed 3 days later, and splenocytes and lymphocytes were collected. After centrifugation at 1500 rpm, the supernatant was discarded and ACK lysis buffer (purchased from Gibco, Cat. A1049201) was added to the cells to lyse the doped red blood cells in the cells to obtain a cell suspension. The cells were washed 3 times with DMEM basic medium (purchased from Gibco, catalog number: 10569044) at 1500 revolutions per minute, and then subjected to cell fusion with mouse myeloma cells SP2/0 (purchased from ATCC, catalog number: CRL-1581) at a ratio of 2:1 of viable cells using electrofusion method (the instrument was BTX 2001+). The fused cells were diluted in DMEM medium contain 20% (v/v) fetal calf serum (purchased from ExCell Bio, catalog number: FND500), 1×HAT (purchased from sigma, catalog number: H0262-10VL), bovine insulin (purchased from Yeason, catalog number: 40107ES25) and NEAA (purchased from Gibco, catalog number: 11140050), then added into a 96-well cell culture plate at 5×10⁴ cells/200 μL per well, and the plate was put in an incubator at 5% (v/v) CO₂ and 37° C. for culture. After 7 days, ELISA was used to screen the supernatant of the fusion plate to determine the binding activity with human BCMA-His protein. For the positive clone supernatant, the binding activity with monkey BCMA-His protein was determined by ELISA, the binding activity with Flp-in CHO-human BCMA was determined by FACS, and the ligand binding competition ELISA was used to detect the blocking effect of hybridoma supernatant on the binding of ligand APRIL to human BCMA protein.

According to the screening results, the qualified positive clones were selected and subcloned with semi-solid medium (purchased from stemcell, catalog number: 03810). 7 days later, the grown clones were picked into a 96-well culture plate one by one, and scale-up cultured in DMEM medium containing 10% (w/w) fetal bovine serum and 1×HT (purchased from sigma, catalog number: H0137-10VL). 1 day later, ELISA was used for preliminary screening, and single clones with binding activity with human BCMA protein were picked to a 24-well plate and amplified for further culture. 3 days later, the culture supernatant was further tested using FACS to evaluate its binding activity with Flp-in CHO-human BCMA and Flp-in CHO-monkey BCMA cells. At the same time, negative cell Flp-in CHO was used to perform FACS to confirm the binding specificity. Based on the test results of the samples in the 24-well plate, the optimal clones were selected and scale-up cultured in DMEM medium containing 10% (v/v) FBS at 37° C. and 5% (v/v) CO₂, and then frozen and stored in liquid nitrogen to obtain the optimal hybridoma cells, which can be used for subsequent antibody production and purification.

The hybridoma supernatant was subjected to scale-up culture, then purified by protein A, and then SFM mouse antibodies were prepared. Murine antibodies whose binding activity was equivalent to or better than that of the positive control were screened by ELISA detection of binding to human BCMA and monkey BCMA recombinant proteins, respectively, by FACS detection of binding to H929 endogenous cells, by SPR detection of affinity with human BCMA recombinant proteins, and by ELISA detection of competitive binding of APRIL or BAFF to BCMA.

After a series of physicochemical and binding activity identifications, multiple candidate murine antibody molecules with equivalent or better physicochemical and functional activities than the positive control antibody were obtained, and the excellent cell strains were selected for subsequent chimeric antibody preparation, identification, and humanization.

### Example 3 Determination of Amino Acid Sequence of Variable Regions of Light and Heavy Chains of Positive Hybridoma Clones

The hybridoma cells in the logarithmic growth phase were collected, and were fully lysed with Trizol (purchased from Invitrogen, catalog number: 15596-018) and stored at −80° C. for testing. Amino acid sequences of variable regions of light and heavy chains of positive hybridoma clones were determined. The sequencing results were analyzed by MOE software, and the phylogenetic tree was constructed according to the amino acid sequence of the protein encoded by the variable region. After eliminating the sequences that were close to each other on the phylogenetic tree according to the sequence similarity, multiple clones were obtained through screening. Antibody CDRs sequences are classified using the Kabat and IMGT numbering systems.

### Example 4 Construction, Expression and Purity Determination of Human-Mouse Chimeric Antibody (scFv-Fc)

The VH and VL fragments of murine antibody molecules were linked through a linker composed of 15 flexible amino acids (GGGGSGGGGSGGGGS) to form scFv, and fused to human Fc to construct a human-mouse chimeric scFv-Fc eukaryotic expression vector (for the human Fc sequence, see SEQ ID NO: 6). Expi 293F cells in the logarithmic growth phase were transiently transfected with polyethylenimine (PEI) (purchased from Sigma-Aldrich, catalog number: 408727). The culture supernatant was collected 5-7 days after transfection and purified by Protein A affinity. The purity of the obtained antibody was qualitatively analyzed by SEC-HPLC. The results are shown in Table 6. The specific purification and analysis methods are as follows:

Experimental reagents and consumables:
(1) Mab affinity equilibration buffer: 1×PBS Buffer;
(2) Mab affinity elution liquid: Dibasic sodium phosphate anhydrous: 2.69 g/L; Sodium dihydrogen phosphate dihydrate: 0.17 g/L; Sodium chloride: 58.44 g/L;
(3) Mab affinity eluent: Citric acid monohydrate: 3.92 g/L; Trisodium citrate dihydrate: 1.88 g/L;
(4) 1M Tris-HCl;
(5) Affinity chromatography packing: Mabselect SuRe™ (No. 10054120, purchased from GE Healthcare).

The packing was resuspended using an appropriate amount of Mab affinity equilibration buffer, then added to the culture supernatant and incubated at 4° C. for 3 h on an orbital shaker (TS-200, Haimen Kylin-Bell Lab Instruments Co., Ltd.). After incubation, the product was added to a gravity empty column, and Mab affinity equilibration buffer, Mab affinity elution liquid, and Mab affinity equilibration buffer were used for rinsing over 5 column volumes. Mab affinity eluent was used for elution and an appropriate amount of 1M Tris-HCl was added to adjust the pH to 5-6. Concentration was determined with an ultramicrovolume spectrophotometer (Nanodrop8000, purchased from Thermo).

Protein samples to be detected were analyzed by SEC-HPLC to characterize the molecular size homogeneity and purity of the proteins. The HPLC used was Agilent 1260, the chromatographic column TSKgel G3000SWXL was from Tosoh Bioscience, the mobile phase was 200 mM phosphate buffer (pH 7.0)/isopropanol (v/v 9:1) (batch number: 20210519001), the detection temperature was 25° C., the flow rate was 0.5 mL/min, and the detection wavelength was 280 nm. For the SEC-HPLC data, the chromatogram was analyzed by a manual integration method, and the protein purity was calculated by an area normalization method. The main peak was considered as a monomer, the chromatographic peak before the main peak was called an aggregate, and the chromatographic peak after the main peak was called a fragment. The results are shown in Table 6. Except for BCMA-mAb03 and BCMA-mAb04, the SEC purity of other antibodies was relatively high.

TABLE 6
Results of BCMA human-mouse chimeric antibody
(scFv-Fc) expression quantity and SEC purity

| | | Expres- | | | SEC |
|---|---|---|---|---|---|
| | | sion | Concen- | | monomer |
| Designation | Expression | volume | tration | Volume | purity |
| of antibody | system | (ml) | (mg/ml) | (ml) | (%) |
| BCMA-mAb01 | expi 293F | 40 | 2.63 | 2 | 96.19 |
| BCMA-mAb02 | expi 293F | 40 | 1.54 | 2 | 98.99 |
| BCMA-mAb03 | expi 293F | 40 | 2.86 | 2 | 60.33 |
| BCMA-mAb04 | expi 293F | 40 | 1.25 | 2 | 84.63 |
| BCMA-mAb05 | expi 293F | 40 | 1.68 | 2 | 90.05 |
| BCMA-mAb06 | expi 293F | 40 | 2.19 | 2 | 93.91 |
| BCMA-mAb07 | expi 293F | 40 | 0.34 | 2 | 96.2 |

### Example 5 Identification of Human-Mouse Chimeric scFv-Fc Antibodies

### (a) Detection of Binding of BCMA Human-Mouse Chimeric Antibodies to Human BCMA Protein and Cross-Binding of BCMA Human-Mouse Chimeric Antibodies to Monkey BCMA Protein by Enzyme-Linked Immunosorbent Assay (ELISA)

The specific method was as follows: Human BCMA protein (purchased from Acro, catalog number: BCA-H522y) and monkey BCMA protein (purchased from Acro, catalog number: BCA-C52H7) were diluted with PBS to a final concentration of 1 μg/mL, respectively, and then added to a 96-well ELISA plate at 50 μl/well. The plate was sealed with a plastic film and incubated overnight at 4° C., the plate was washed twice with PBS the next day, and then a blocking solution [PBS+2% (w/v) BSA] was added for blocking at room temperature for 2 h. The blocking solution was discarded, the plate was washed 3 times with PBS, and 100 nM BCMA human-mouse chimeric antibody diluted gradiently or control antibody was added at 50 μl/well. After incubation at 37° C. for 2 h, the plate was washed 3 times with PBS. HRP (horseradish peroxidase)-labeled secondary antibody (purchased from Merck, catalog number: AP113P) was added, and incubated at 37° C. for 1 h, and the plate was washed 5 times with PBS. TMB substrate was added at 50 μl/well, and incubated at room temperature for 10 min, then a stop solution (1.0 M HCl) was added at 50 μl/well. The OD450 nm values were read by an ELISA microplate reader (Multimode Plate Reader, EnSight, purchased from Perkin Elmer). The results are shown in Table 7, Table 8 and FIGS. 7A-7B and FIG. 8A-8B. All chimeric antibodies have good binding activity with human BCMA protein; except that BCMA-mAb06 has no cross-binding activity with monkey BCMA protein and BCMA-mAb07 has weak binding to monkey BCMA protein, other chimeric antibodies have good binding activity with monkey BCMA protein. The IgG subtype control is human IgG1.

TABLE 7
ELISA detection of binding activity of chimeric
antibodies with human BCMA protein

| | Antibody concentration (nM) | | | | | | | |
|---|---|---|---|---|---|---|---|---|
| Designation | | | | | | | | Blank |
| of antibody | 100 | 10 | 1 | 0.1 | 0.01 | 0.001 | 0.0001 | control |
| BCMA-mAb01 | 2.89 | 2.52 | 1.76 | 0.43 | 0.10 | 0.06 | 0.07 | 0.06 |
| BCMA-mAb02 | 2.77 | 2.42 | 1.01 | 0.24 | 0.06 | 0.05 | 0.05 | 0.06 |
| BCMA-mAb03 | 3.20 | 2.51 | 1.00 | 0.21 | 0.09 | 0.07 | 0.06 | 0.07 |
| BCMA-mAb04 | 2.63 | 2.59 | 1.31 | 0.26 | 0.11 | 0.08 | 0.07 | 0.06 |
| REGN5459- | 2.84 | 2.62 | 2.16 | 0.90 | 0.31 | 0.17 | 0.10 | 0.06 |
| hIgG1 | | | | | | | | |
| HPN217-hHcAb | 2.77 | 2.66 | 2.33 | 1.40 | 0.54 | 0.26 | 0.17 | 0.09 |
| hIgG1 | 0.06 | 0.27 | 0.06 | 0.06 | 0.06 | 0.06 | 0.06 | 0.07 |
| BCMA-mAb05 | 2.14 | 2.73 | 2.25 | 1.20 | 0.31 | 0.12 | 0.08 | 0.09 |
| BCMA-mAb06 | 2.53 | 2.52 | 2.26 | 1.29 | 0.40 | 0.12 | 0.09 | 0.09 |
| BCMA-mAb07 | 2.29 | 2.21 | 2.29 | 1.46 | 0.43 | 0.14 | 0.08 | 0.08 |
| REGN5459- | 2.79 | 2.53 | 2.42 | 1.56 | 0.47 | 0.14 | 0.09 | 0.08 |
| hIgG1 | | | | | | | | |
| HPN217-hHcAb | 2.68 | 2.60 | 2.41 | 2.21 | 0.87 | 0.44 | 0.18 | 0.07 |
| hIgG1 | 0.27 | 0.12 | 0.21 | 0.23 | 0.23 | 0.21 | 0.09 | 0.07 |

TABLE 8
ELISA detection of cross-binding activity of
chimeric antibodies with monkey BCMA protein

| | Antibody concentration (nM) | | | | | | | |
|---|---|---|---|---|---|---|---|---|
| Designation | | | | | | | | Blank |
| of antibody | 100 | 10 | 1 | 0.1 | 0.01 | 0.001 | 0.0001 | control |
| BCMA-mAb01 | 2.73 | 2.60 | 2.08 | 0.82 | 0.19 | 0.07 | 0.06 | 0.06 |
| BCMA-mAb02 | 2.56 | 2.55 | 1.32 | 0.28 | 0.08 | 0.05 | 0.05 | 0.06 |
| BCMA-mAb03 | 3.06 | 2.58 | 1.73 | 0.55 | 0.19 | 0.10 | 0.08 | 0.05 |
| BCMA-mAb04 | 2.95 | 2.66 | 1.85 | 0.68 | 0.27 | 0.15 | 0.10 | 0.06 |
| REGN5459-hIgG1 | 2.83 | 3.06 | 2.71 | 2.16 | 1.22 | 0.66 | 0.37 | 0.06 |
| HPN217-hHcAb | 0.08 | 0.06 | 0.05 | 0.06 | 0.06 | 0.07 | 0.06 | 0.06 |
| hIgG1 | 0.08 | 0.08 | 0.06 | 0.08 | 0.06 | 0.06 | 0.06 | 0.06 |
| BCMA-mAb05 | 2.99 | 2.71 | 1.87 | 0.43 | 0.10 | 0.06 | 0.05 | 0.06 |
| BCMA-mAb06 | 0.81 | 0.32 | 0.15 | 0.10 | 0.07 | 0.05 | 0.05 | 0.06 |
| BCMA-mAb07 | 1.98 | 1.10 | 0.38 | 0.11 | 0.06 | 0.05 | 0.05 | 0.05 |
| REGN5459-hIgG1 | 3.15 | 2.94 | 2.80 | 1.59 | 0.43 | 0.14 | 0.07 | 0.06 |
| HPN217-hHcAb | 0.30 | 0.15 | 0.13 | 0.12 | 0.17 | 0.16 | 0.05 | 0.05 |
| hIgG1 | 0.18 | 0.06 | 0.17 | 0.19 | 0.19 | 0.17 | 0.08 | 0.05 |

### (B) Cell-Based ELISA Detection of the Binding of BCMA Human-Mouse Chimeric Antibodies to Flp-inCHO-Human BCMA Cells

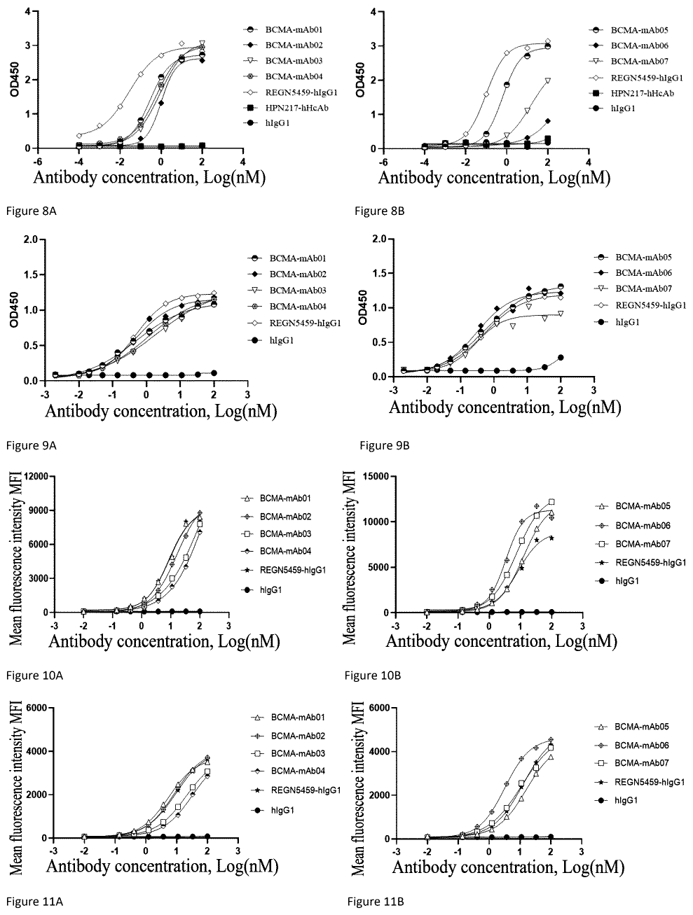

The specific method was as follows: The required cells were scale-up cultured in a T-75 cell culture flask to the logarithmic growth phase, the medium was aspirated, the cells were washed twice with a PBS buffer, and then digested with trypsin. Then the digestion was terminated with a complete medium, and the cells were pipetted to obtain a single cell suspension. After the cells were counted and centrifuged, the cell precipitate was resuspended to 4×10⁵ cells per milliliter with a complete medium, and the suspension was added to a 96-well flat-bottomed cell culture plate at 100 μl/well, and cultured overnight in a 37° C., 5% carbon dioxide incubator. On the next day, the medium in the 96-well plate was discarded, the cells were washed once with PBS, and then fixed at room temperature for 0.5 h with an immunostaining fixative (purchased from Beyotime, catalog number: P0098-500 ml) at 50 μl/well. A blocking solution [PBS+5% (w/v) skim milk] was added for blocking at room temperature for 2 h. The blocking solution was discarded, the plate was washed 3 times with PBS, and 100 nM BCMA human-mouse chimeric antibody diluted gradiently or control antibody was added at 50 μl/well. After incubation at 37° C. for 2 h, the plate was washed 3 times with PBS. HRP (horseradish peroxidase)-labeled secondary antibody (purchased from Merck, catalog number: AP113P) was added, and incubated at 37° C. for 1 h, and the plate was washed 5 times with PBS. TMB substrate was added at 50 μl/well, and incubated at room temperature for 10 min, then a stop solution (1.0 M HCl) was added at 50 μl/well. The OD450 nm values were read by an ELISA microplate reader (Multimode Plate Reader, EnSight, purchased from Perkin Elmer). The results are shown in Table 9 and FIGS. 9A-9B. The chimeric antibodies bind well to Flp-inCHO-human BCMA cells. The IgG subtype control is human IgG1.

TABLE 9
Cell-based ELISA detection of the binding activity of chimeric
antibodies with Flp-inCHO-human BCMA cells (nM)

| | Antibody concentration (nM) | | | | | | | | | | | |
|---|---|---|---|---|---|---|---|---|---|---|---|---|
| Designation | | | | | | | | | | | | Blank |
| of antibody | 100.00 | 33.33 | 11.11 | 3.70 | 1.23 | 0.41 | 0.14 | 0.05 | 0.02 | 0.01 | 0.002 | control |
| BCMA- | 1.08 | 1.05 | 0.90 | 0.86 | 0.70 | 0.56 | 0.40 | 0.22 | 0.13 | 0.10 | 0.09 | 0.08 |
| mAb01 | | | | | | | | | | | | |
| BCMA- | 1.18 | 1.11 | 1.06 | 0.91 | 0.87 | 0.54 | 0.36 | 0.18 | 0.11 | 0.09 | 0.08 | 0.08 |
| mAb02 | | | | | | | | | | | | |
| BCMA- | 1.15 | 1.07 | 0.87 | 0.73 | 0.67 | 0.40 | 0.28 | 0.19 | 0.11 | 0.09 | 0.08 | 0.08 |
| mAb03 | | | | | | | | | | | | |
| BCMA- | 1.15 | 1.09 | 0.94 | 0.81 | 0.68 | 0.42 | 0.32 | 0.18 | 0.11 | 0.07 | 0.07 | 0.07 |
| mAb04 | | | | | | | | | | | | |
| REGN5459- | 1.24 | 1.20 | 1.17 | 1.09 | 0.88 | 0.61 | 0.35 | 0.18 | 0.12 | 0.09 | 0.08 | 0.08 |
| hIgG1 | | | | | | | | | | | | |
| hIgG1 | 0.11 | 0.09 | 0.08 | 0.08 | 0.08 | 0.08 | 0.08 | 0.08 | 0.08 | 0.08 | 0.08 | 0.08 |
| BCMA- | 1.31 | 1.24 | 1.17 | 1.01 | 0.84 | 0.65 | 0.43 | 0.22 | 0.15 | 0.10 | 0.09 | 0.07 |
| mAb05 | | | | | | | | | | | | |
| BCMA- | 1.21 | 1.21 | 1.28 | 0.96 | 0.99 | 0.74 | 0.44 | 0.27 | 0.15 | 0.10 | 0.09 | 0.08 |
| mAb06 | | | | | | | | | | | | |
| BCMA- | 0.91 | 0.84 | 1.01 | 0.73 | 0.76 | 0.60 | 0.31 | 0.19 | 0.12 | 0.10 | 0.10 | 0.08 |
| mAb07 | | | | | | | | | | | | |
| REGN5459- | 1.15 | 1.16 | 1.12 | 1.00 | 0.77 | 0.57 | 0.40 | 0.21 | 0.14 | 0.10 | 0.09 | 0.08 |
| hIgG1 | | | | | | | | | | | | |
| hIgG1 | 0.28 | 0.14 | 0.10 | 0.09 | 0.09 | 0.09 | 0.09 | 0.09 | 0.10 | 0.09 | 0.09 | 0.07 |

### (C) Flow Cytometry Experiment (FACS) Detection of Binding of Chimeric Antibodies to Endogenous Cells Expressing BCMA

The desired cells were scale-up cultured in T-75 cell culture flasks to the logarithmic growth phase, and the cells were pipetted into a single cell suspension. After the cells were counted, the cells were centrifuged and the cell pellet was resuspended in FACS buffer (PBS+2% fetal calf serum) to 2×10⁶ cells per ml, and the suspension was added to the 96-well FACS reaction plate at 50 μl per well, then the sample to be tested of the chimeric antibody was added at 50 μl per well and the mixture was incubated at 4° C. for 1 h. The obtained mixture was centrifuged and washed 3 times with a PBS buffer. Secondary antibody anti-hIgG (Fc) Alexa 647 (purchased from Jackson, catalog number: 109-605-098) was added at 50 μl/well, and incubated on ice for 1 h. The obtained mixture was centrifuged and washed 3 times with a PBS buffer. 100 μl of the mixture was taken for detection and result analysis by FACS (FACS Canto™, purchased from BD Company). Data analysis was performed by software (FlowJo) to obtain the mean fluorescence intensity (MFI) of the cells. Then, a software (GraphPad Prism8) was used for analysis, data fitting and EC50 calculation. The results are shown in Table 10 and FIGS. 10A-10B, FIGS. 11A-11B, and FIGS. 12A-12B. All seven chimeric antibodies bind to three types of BCMA-expressing endogenous cells.

TABLE 10
FACS detection of binding activity of chimeric
antibodies with H929, U266 and RPMI8226 cells

| | H929 | | U266 | | RPMI8226 | |
|---|---|---|---|---|---|---|
| | Maximum | | Maximum | | Maximum | |
| | mean | | mean | | mean | |
| Designation | fluorescence | EC50 | fluorescence | EC50 | fluorescence | EC50 |
| of antibody | intensity | (nM) | intensity | (nM) | intensity | (nM) |
| BCMA-mAb01 | 8386 | 9.02 | 3499 | 5.46 | 1584 | 3.30 |
| BCMA-mAb02 | 8772 | 16.46 | 3716 | 8.51 | 1431 | 4.63 |
| BCMA-mAb03 | 7787 | 104.00 | 3068 | 20.51 | 1487 | 11.27 |
| BCMA-mAb04 | 7071 | 166.80 | 2852 | 32.20 | 1291 | 25.96 |
| REGN5459-hIgG1 | 8191 | 8.39 | 3634 | 9.52 | 1296 | 6.79 |
| hIgG1 | 94 | Negative | 86 | Negative | 81 | Negative |
| BCMA-mAb05 | 11018 | 12.38 | 3755 | 16.00 | 2232 | 12.11 |
| BCMA-mAb06 | 11713 | 3.39 | 4550 | 3.23 | 2858 | 2.28 |
| BCMA-mAb07 | 12179 | 7.45 | 4175 | 11.32 | 2220 | 6.38 |
| REGN5459-hIgG1 | 11612 | 7.74 | 4313 | 12.84 | 2547 | 4.52 |
| hIgG1 | 69 | Negative | 101 | Negative | 107 | Negative |

### (D) Ligand Binding Competition ELISA Detection of the Blocking Effect of Chimeric Antibodies on the Binding of Ligand APRIL to Human BCMA Protein

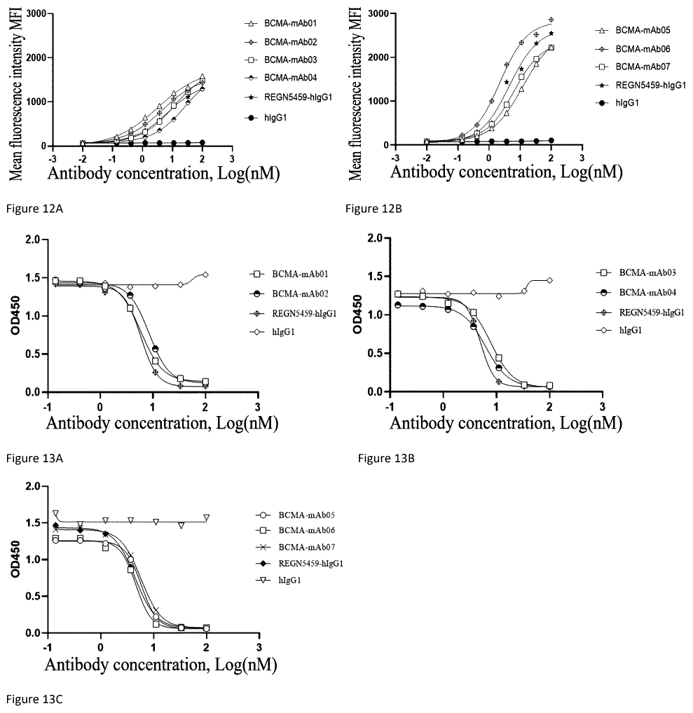

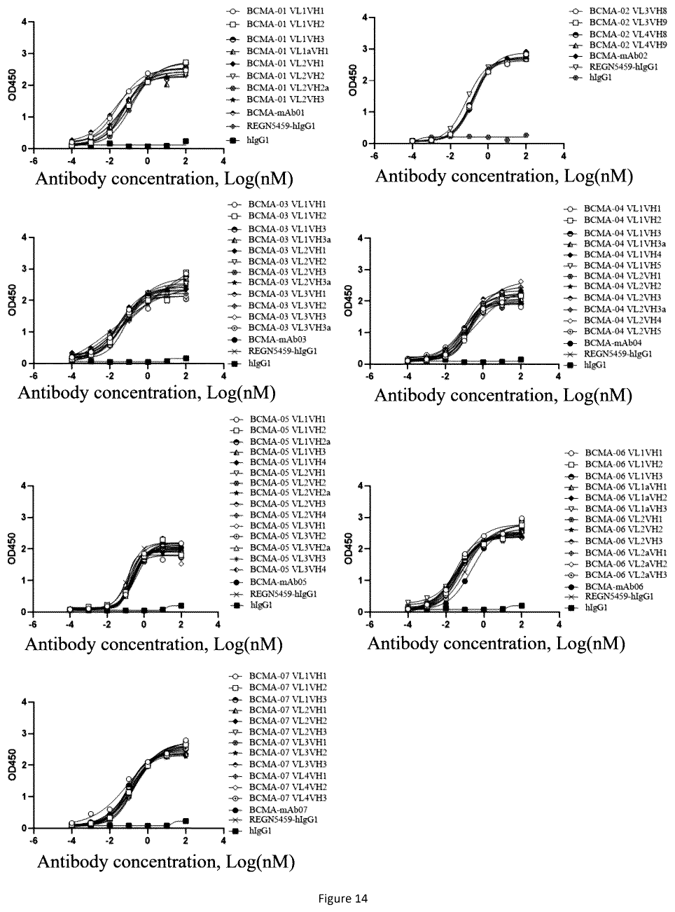

The antigen protein was diluted with PBS to a final concentration of 1 μg/mL, and then added to a 96-well ELISA plate at 50 μl per well. The plate was sealed with a plastic film and incubated overnight at 4° C., the plate was washed twice with PBS the next day, and then a blocking solution [PBS+2% (w/v) BSA] was added for blocking at room temperature for 2 h. The blocking solution was discarded, the plate was washed 3 times with PBS, and 200 nM BCMA human-mouse chimeric antibody diluted gradiently or negative control antibody was added at 50 μl/well. hAPRIL-biotin (purchased from Acro, catalog number: APL-H82F5) was diluted to 0.5 μg/mL, with 50 μl per well. After incubation at 37° C. for 1.5 h, the plate was washed 3 times with PBS. HRP (horseradish peroxidase)-labeled secondary antibody (purchased from Sigma, catalog number: S2438-250 ug) was added, and incubated at 37° C. for 1 h, and the plate was washed 5 times with PBS. TMB substrate was added at 50 μl/well, and incubated at room temperature for 10 min, then a stop solution (1.0 M HCl) was added at 50 μl/well. The OD450 nm values were read by an ELISA microplate reader (Multimode Plate Reader, EnSight, purchased from Perkin Elmer). The results are shown in Table 11 and FIGS. 13A-13C. The results show that the 7 antibodies to be tested can well block the binding of the ligand APRIL to human BCMA protein.

TABLE 11
Ligand binding competition ELISA detection of the blocking effect of chimeric
antibodies on the binding of ligand APRIL to human BCMA protein

| Designation | Antibody concentration (nM) | | | | | | | |
|---|---|---|---|---|---|---|---|---|
| of antibody | 100.00 | 33.33 | 11.11 | 3.70 | 1.23 | 0.41 | 0.14 | 0.00 |
| BCMA-mAb01 | 0.14 | 0.18 | 0.41 | 1.10 | 1.39 | 1.46 | 1.46 | 1.55 |
| BCMA-mAb02 | 0.12 | 0.18 | 0.57 | 1.27 | 1.36 | 1.44 | 1.47 | 1.55 |
| REGN5459-hIgG1 | 0.08 | 0.08 | 0.26 | 1.11 | 1.31 | 1.42 | 1.42 | 1.50 |
| hIgG1 | 1.54 | 1.41 | 1.39 | 1.38 | 1.43 | 1.41 | 1.43 | 1.52 |
| BCMA-mAb03 | 0.08 | 0.09 | 0.44 | 1.03 | 1.15 | 1.24 | 1.27 | 1.39 |
| BCMA-mAb04 | 0.07 | 0.09 | 0.31 | 0.85 | 1.07 | 1.11 | 1.13 | 1.30 |
| REGN5459-hIgG1 | 0.07 | 0.07 | 0.13 | 0.92 | 1.16 | 1.24 | 1.27 | 1.36 |
| hIgG1 | 1.45 | 1.31 | 1.24 | 1.29 | 1.27 | 1.31 | 1.28 | 1.45 |
| BCMA-mAb05 | 0.06 | 0.07 | 0.22 | 1.00 | 1.22 | 1.26 | 1.26 | 1.59 |
| BCMA-mAb06 | 0.07 | 0.07 | 0.12 | 0.86 | 1.16 | 1.29 | 1.29 | 1.58 |
| BCMA-mAb07 | 0.08 | 0.09 | 0.31 | 1.06 | 1.34 | 1.43 | 1.41 | 1.66 |
| REGN5459-hIgG1 | 0.07 | 0.09 | 0.22 | 0.90 | 1.35 | 1.40 | 1.47 | 1.54 |
| hIgG1 | 1.57 | 1.46 | 1.51 | 1.53 | 1.53 | 1.48 | 1.63 | 1.72 |

### Example 6 Detection of Affinity of BCMA Human-Mouse Chimeric Antibodies

Anti-BCMA human-mouse chimeric antibodies were captured using a Protein A chip (GE Helthcare; 29-127-558). A sample buffer and a running buffer were HBS-EP+ (10 mM HEPES, 150 mM NaCl, 3 mM EDTA and 0.05% surfactant P20) (GE Healthcare; BR-1006-69). The flow-through cell was set to 25° C. The sample block was set to 16° C. Both were pretreated with the running buffer. In each cycle, an antibody to be tested was first captured with a Protein A chip, and then a single concentration of BCMA antigen protein was injected. The binding and dissociation processes of the antibody and the antigen protein were recorded, and finally Glycine pH 1.5 (GE Helthcare; BR-1003-54) was used to complete chip regeneration. Binding was measured by injecting different concentrations of BCMA-His in solution for 240 s with a flow rate of 30 μL/min. The concentration started from 200 nM (see the detailed results for the actual concentration in the test) and was diluted at 1:1, making a total of 5 concentrations. The dissociation phase was monitored for up to 600 s and was triggered by switching from sample solution to running buffer. The surface was regenerated by washing with a 10 mM glycine solution (pH 1.5) for 30 s at a flow rate of 30 μL/min. Bulk refractive index difference was corrected by subtracting the response obtained from the goat anti-human Fc surface. Blank injection was also subtracted (=double reference). For calculation of apparent KD and other kinetic parameters, Langmuir 1:1 model was used. The binding rate (Kₐ), dissociation rate (K_{dis}) and binding affinity (KD) of the chimeric antibodies to human BCMA protein are shown in the table, wherein REGN5459-hlgG1 and HPN217-hHcAb antibodies were used as controls. As shown in Table 12, the KD values of all the chimeric antibodies for human BCMA protein are below 1E-9M.

TABLE 12
Binding affinity of chimeric antibodies to human BCMA protein

| Designation of antibody | Ka (1/Ms) | Kdis (1/s) | KD (M) |
|---|---|---|---|
| BCMA-mAb01 | 1.68E+06 | 8.20E−04 | 4.87E−10 |
| BCMA-mAb02 | 1.29E+06 | 2.53E−04 | 1.96E−10 |
| BCMA-mAb03 | 1.16E+06 | 1.46E−04 | 1.26E−10 |
| BCMA-mAb04 | 1.13E+06 | 1.06E−04 | 9.35E−11 |
| BCMA-mAb05 | 1.15E+06 | 2.96E−05 | 2.58E−11 |
| BCMA-mAb06 | 8.85E+05 | 3.53E−04 | 3.98E−10 |
| BCMA-mAb07 | 1.34E+06 | 1.06E−03 | 7.92E−10 |
| REGN5459-hIgG1 | 8.50E+05 | 1.70E−04 | 2.00E−10 |
| HPN217-hHcAb | 3.34E+06 | 2.73E−03 | 8.18E−10 |

The variable region sequences of the above 7 chimeric antibodies are shown in Table 13, and the CDR sequences corresponding to Kabat and IMGT numbers are shown in Table 14.

TABLE 13
Amino acid sequences of variable regions of chimeric antibodies

| Designation | | |
|---|---|---|
| of | | |
| antibody | No. | Amino acid sequence |
| BCMA-mab01 | SEQ ID | EVKLVESGGGLVQPGGSLKLSCAASGFTFSDFYMYWVRQTP |
| VH | NO: 7 | EKRLEWVAYITNGGENTYFPDTVKGRFTISRDNAKNTLYLQM |
| | | SRLKSEDTAMYYCARQNPFYYYDSNYDYAMDFWGQGTSVT |
| | | VSS |
| BCMA-mab01 | SEQ ID | DVVMTQTPLTLSVTIGQPASISCKSSQSLLYSNGKTYLSWLFQ |
| VL | NO: 8 | RPGQSPKRLIYLVSELDSGVPDRFTGSGSGTDFTLKISRVEA |
| | | EDLGIYYCVQGTHFPHTFGGGTKLEIK |
| BCMA-mab02 | SEQ ID | EVQLVESGGDLVQPGESLKLSCESNAYEFPSYDMSWVRKTP |
| VH | NO: 9 | EKRLELVAAINSDGGITYYPDTMERRFIISRDNTKKTLYLQMSS |
| | | LRSEDTALYYCARHGYGGGAYVLDYWGQGTSVTVSS |
| BCMA-mab02 | SEQ ID | DIVMTQSQKFMSTTVGDRVRITCKASQNVGTAVAWYQQKPG |
| VL | NO: 10 | QSPKVLIYSASSRYTGVPDRFTGSGSGTDFTLTINNMQSEDL |
| | | ADYFCQQYSNYHLTFGTGTKLELK |
| BCMA-mab03 | SEQ ID | QIPLVQSGPELKKPGETVKISCKASGYTFTNYAMTWVKQAPG |
| VH | NO: 11 | KGLKWMGWIDTYSGVPTYADDFKGRFAFSLETSATTAYLQIN |
| | | NLKNEDTATYFCARVGVYGGYDTLDSWGQGTSVTVSS |
| BCMA-mab03 | SEQ ID | DIQMTQTTSSLSASLGDRVTISCSASQGISNHLNWYQQKPDG |
| VL | NO: 12 | TVKLLIYYTSSLHSGVPSRFSGSGSGTDYSLTISNLEPEDFAT |
| | | YYCQQYNNLPWTFGGGTKLEIK |
| BCMA- | SEQ ID | QVQLQQSGAELARPGASVDLSCKASGYSFTSYGISWVKLRT |
| mAb04 VH | NO: 13 | GQGLEWIGEIYPRDDITYYNEKFKGKTTLTADKSSSTAYMELR |
| | | SLTSEDSAVYFCARHDYYYVNSVSAMDYWGQGTSVTVSS |
| BCMA- | SEQ ID | DIVMSQSPSSLAVSVGEKVTMSCKSSQSLLYSSNQKNYLAW |
| mAb04 VL | NO: 14 | YQQKPGQSPKLLIYWASTRYSGVPDRFTGSGSGTDFTLTVS |
| | | SVRAEDLALYYCQQYYIYPWTFGGGTKLEIK |
| BCMA-mab05 | SEQ ID | QGQMQQSDAELVKPGASVQISCKASGYMFTDHTFHWMRQR |
| VH | NO: 15 | PEQGLEWIGYIYPRDDNTKYNEKFKGKATLTADKSSSTAYIHL |
| | | NSLTSEDSAVYFCARAGYYGSSHAMDYWGQGTSVTVSS |
| BCMA-mab05 | SEQ ID | DIVMTQSHKFMSTSVGDRVSITCKASQDVSTAVAWYQQKPG |
| VL | NO: 16 | QSPRLLIYSASSRYTGVPDRFTGSGSGTDFTFTISSVQTEDLA |
| | | VYYCQQLYSTPWTFGGGTKLEIK |
| BCMA-mab06 | SEQ ID | EVKLLQSGGGLVQPGGSLKLSCAASGIDFSRYWMSWVRRA |
| VH | NO: 17 | PGKGLEWIGEINPDSSTINYAPSLKDEFIISRDNAKNTLYLQMS |
| | | KVRSEDTALYYCAIFYYANYYAMDYWGQGTSVTVSS |
| BCMA-mab06 | SEQ ID | DIVMTQSQKFMSTSVGDRVSVTCKASQNVDTNVAWYQQKP |
| VL | NO: 18 | GQSPKALIYSASYRFSGVPDRFTGSGSGTDFTLTISNVQSED |
| | | LAEYFCQQYNGYPFTFASGTKLEIK |
| BCMA-mab07 | SEQ ID | QVQLQQSGAELAKPGASVKLSCKASGYTFTDYWTHWVKQR |
| VH | NO: 19 | PGQGLEWIGYINHGSGYTKYNQKFKDKATLTADKSSSTAYMQ |
| | | VSSLTYEDSAVYYCARLTGSYYFDYWGQGTTLTVSS |
| BCMA-mab07 | SEQ ID | QIVLTQSPAIMSASPGEKVTMTCSASSSVSYMHWYQQKSGT |
| VL | NO: 20 | SPKRWIYDTSKLASGVPPRFSGSGSGTSYSLTISSMEAEDAA |
| | | TYYCQQWSSNPLTFGAGTKLELK |

TABLE 14
Analysis of CDR sequences by Kabat and IMGT numbering

| Designation | Method | | | |
|---|---|---|---|---|
| of | for | | | |
| antibody | division | CDR1 | CDR2 | CDR3 |
| BCMA- | Kabat | DFYMY | YITNGGENTYFPD | QNPFYYYDSNYDY |
| mab01 VH | | (SEQ ID NO: 101) | TVKG | AMDF |
| | | | (SEQ ID NO: 102) | (SEQ ID NO: 103) |
| | IMGT | GFTFSDFY | ITNGGENT | ARQNPFYYYDSNY |
| | | (SEQ ID NO: 104) | (SEQ ID NO: 105) | DYAMDF |
| | | | | (SEQ ID NO: 106) |
| BCMA- | Kabat | KSSQSLLYSNGKT | LVSELDS | VQGTHFPHT |
| mab01 VL | | YLS | (SEQ ID NO: 108) | (SEQ ID NO: 109) |
| | | (SEQ ID NO: 107) | | |
| | IMGT | QSLLYSNGKTY | LVS | VQGTHFPHT |
| | | (SEQ ID NO: 110) | (SEQ ID NO: 111) | (SEQ ID NO: 109) |
| BCMA- | Kabat | SYDMS | AINSDGGITYYPDT | HGYGGGAYVLDY |
| mab02 VH | | (SEQ ID NO: 112) | MER | (SEQ ID NO: 114) |
| | | | (SEQ ID NO: 113) | |
| | IMGT | AYEFPSYD | INSDGGIT | ARHGYGGGAYVL |
| | | (SEQ ID NO: 115) | (SEQ ID NO: 116) | DY |
| | | | | (SEQ ID NO: 117) |
| BCMA- | Kabat | KASQNVGTAVA | SASSRYT | QQYSNYHLT |
| mab02 VL | | (SEQ ID NO: 118) | (SEQ ID NO: 119) | (SEQ ID NO: 120) |
| | IMGT | QNVGTA | SAS | QQYSNYHLT |
| | | (SEQ ID NO: 121) | (SEQ ID NO: 122) | (SEQ ID NO: 120) |
| BCMA- | Kabat | NYAMT | WIDTYSGVPTYAD | VGVYGGYDTLDS |
| mab03 VH | | (SEQ ID NO: 123) | DFKG | (SEQ ID NO: 125) |
| | | | (SEQ ID NO: 124) | |
| | IMGT | GYTFTNYA | IDTYSGVP | ARVGVYGGYDTL |
| | | (SEQ ID NO: 126) | (SEQ ID NO: 127) | DS |
| | | | | (SEQ ID NO: 128) |
| BCMA- | Kabat | SASQGISNHLN | YTSSLHS | QQYNNLPWT |
| mab03 VL | | (SEQ ID NO: 129) | (SEQ ID NO: 130) | (SEQ ID NO: 131) |
| | IMGT | QGISNH | YTS | QQYNNLPWT |
| | | (SEQ ID NO: 132) | (SEQ ID NO: 133) | (SEQ ID NO: 131) |
| BCMA- | Kabat | SYGIS | EIYPRDDITYYNEK | HDYYYVNSVSAM |
| mAb04 VH | | (SEQ ID NO: 134) | FKG | DY |
| | | | (SEQ ID NO: 135) | (SEQ ID NO: 136) |
| | IMGT | GYSFTSYG | IYPRDDIT | ARHDYYYVNSVSA |
| | | (SEQ ID NO: 137) | (SEQ ID NO: 138) | MDY |
| | | | | (SEQ ID NO: 139) |
| BCMA- | Kabat | KSSQSLLYSSNQK | WASTRYS | QQYYIYPWT |
| mAb04 VL | | NYLA | (SEQ ID NO: 141) | (SEQ ID NO: 142) |
| | | (SEQ ID NO: 140) | | |
| | IMGT | QSLLYSSNQKNY | WAS | QQYYIYPWT |
| | | (SEQ ID NO: 143) | (SEQ ID NO: 144) | (SEQ ID NO: 142) |
| BCMA- | Kabat | DHTFH | YIYPRDDNTKYNE | AGYYGSSHAMDY |
| mab05 VH | | (SEQ ID NO: 145) | KFKG | (SEQ ID NO: 147) |
| | | | (SEQ ID NO: 146) | |
| | IMGT | GYMFTDHT | IYPRDDNT | ARAGYYGSSHAM |
| | | (SEQ ID NO: 148) | (SEQ ID NO: 149) | DY |
| | | | | (SEQ ID NO: 150) |
| BCMA- | Kabat | KASQDVSTAVA | SASSRYT | QQLYSTPWT |
| mab05 VL | | (SEQ ID NO: 151) | (SEQ ID NO: 152) | (SEQ ID NO: 153) |
| | IMGT | QDVSTA | SAS | QQLYSTPWT |
| | | (SEQ ID NO: 154) | (SEQ ID NO: 155) | (SEQ ID NO: 153) |
| BCMA- | Kabat | RYWMS | EINPDSSTINYAPS | FYYANYYAMDY |
| mab06 VH | | (SEQ ID NO: 156) | LKD | (SEQ ID NO: 158) |
| | | | (SEQ ID NO: 157) | |
| | IMGT | GIDFSRYW | INPDSSTI | AIFYYANYYAMDY |
| | | (SEQ ID NO: 159) | (SEQ ID NO: 160) | (SEQ ID NO: 161) |
| BCMA- | Kabat | KASQNVDTNVA | SASYRFS | QQYNGYPFT |
| mab06 VL | | (SEQ ID NO: 162) | (SEQ ID NO: 163) | (SEQ ID NO: 164) |
| | IMGT | QNVDTN | SAS | QQYNGYPFT |
| | | (SEQ ID NO: 165) | (SEQ ID NO: 166) | (SEQ ID NO: 164) |
| BCMA- | Kabat | DYWTH | YINHGSGYTKYNQ | LTGSYYFDY |
| mab07 VH | | (SEQ ID NO: 167) | KFKD | (SEQ ID NO: 169) |
| | | | (SEQ ID NO: 168) | |
| | IMGT | GYTFTDYW | INHGSGYT | ARLTGSYYFDY |
| | | (SEQ ID NO: 170) | (SEQ ID NO: 171) | (SEQ ID NO: 172) |
| BCMA- | Kabat | SASSSVSYMH | DTSKLAS | QQWSSNPLT |
| mab07 VL | | (SEQ ID NO: 173) | (SEQ ID NO: 174) | (SEQ ID NO: 175) |
| | IMGT | SSVSY | DTS | QQWSSNPLT |
| | | (SEQ ID NO: 176) | (SEQ ID NO: 177) | (SEQ ID NO: 175) |

### Example 7 Humanization of Murine Antibody

By aligning the IMGT (http://imgt.cines.fr) human antibody heavy and light chain variable region germline gene database, heavy chain and light chain variable region germline genes having high homology to the murine antibody were selected as templates, respectively, and the CDRs of the murine antibody were grafted into the corresponding human template, to form a variable region sequence in the order of FR1-CDR1-FR2-CDR2-FR3-CDR3-FR4. According to needs, the key amino acids in the framework sequence were back-mutated to the corresponding amino acids of the murine antibody to ensure the original affinity, and thus the humanized monoclonal antibody was obtained. The CDR amino acid residues of the antibody were determined and annotated by the Kabat numbering system.

### (a) Humanization of BCMA-mAb01 Antibody

The humanization light chain templates of the murine antibody BCMA-mAb01 were IGKV2-30*01 and IGKJ4*01, and the humanization heavy chain templates were IGHV3-7*01 and IGHJ6*01. The CDRs of the murine antibody BCMA-mAb01 were grafted into the human templates of the murine antibody respectively, and thus the corresponding humanized versions were obtained. According to needs, the key amino acids in the FR region sequence of the humanized antibody of BCMA-mAb01 were back-mutated to the corresponding amino acids of the murine antibody to ensure the original affinity. If there are sites prone to chemical modification in the antibody, these sites are subjected to point mutation to eliminate the risk of modification. The specific mutation designs are shown in Table 15.

TABLE 15
Mutation design of humanized antibody of BCMA-mAb01

| VL | | VH | |
|---|---|---|---|
| L1 | Graft(IGKV2-30*01) + | H1 | Graft(IGHV3-7*01) |
| | F41L, Q42F | | |
| L1a | Graft(IGKV2-30*01) + | H2 | Graft(IGHV3-7*01) + |
| | F41L, Q42F + G34A | | G42E, G44R |
| L2 | Graft(IGKV2-30*01) + | H2a | Graft(IGHV3-7*01) + |
| | F41L, Q42F, R50K | | G42E, G44R + G54A |
| | | H3 | Graft(IGHV3-7*01) + |
| | | | Q3K, G42E, G44R |

Notes:
Graft represents the grafting of the CDRs of the murine antibody into the FR region sequence of the human germline template; F41L represents the mutation of F at position 41 of Graft into L, and so on. The numbering of backmutated amino acids is according to the natural sequence numbering.

The specific sequence of the variable region of the humanized antibody of BCMA-mAb01 is as follows:

The amino acid sequence of BCMA-mAb01.
VL1 is as shown in SEQ ID NO: 21:
DVVMTQSPLSLPVTLGQPASISCKSSQSLLYSNGKTYLSWLFQRP
GQSPRRLIYLVSELDSGVPDRFSGSGSGTDFTLKISRVEAEDVGV
YYCVQGTHFPHTFGGGTKVEIK
The amino acid sequence of BCMA-mAb01.
VL1a is as shown in SEQ ID NO: 22:
DVVMTQSPLSLPVTLGQPASISCKSSQSLLYSNAKTYLSWLFQRP
GQSPRRLIYLVSELDSGVPDRFSGSGSGTDFTLKISRVEAEDVGV
YYCVQGTHFPHTFGGGTKVEIK
The amino acid sequence of BCMA-mAb01.
VL2 is as shown in SEQ ID NO: 23:
DVVMTQSPLSLPVTLGQPASISCKSSQSLLYSNGKTYLSWLFQRP
GQSPKRLIYLVSELDSGVPDRFSGSGSGTDFTLKISRVEAEDVGV
YYCVQGTHFPHTFGGGTKVEIK
The amino acid sequence of BCMA-mAb01.
VH1 is as shown in SEQ ID NO: 24:
EVQLVESGGGLVQPGGSLRLSCAASGFTFSDFYMYWVRQAPGKGL
EWVAYITNGGENTYFPDTVKGRFTISRDNAKNSLYLQMNSLRAED
TAVYYCARQNPFYYYDSNYDYAMDFWGQGTTVTVSS
The amino acid sequence of BCMA-mAb01.
VH2 is as shown in SEQ ID NO: 25:
EVQLVESGGGLVQPGGSLRLSCAASGFTFSDFYMYWVRQAPEKRL
EWVAYITNGGENTYFPDTVKGRFTISRDNAKNSLYLQMNSLRAED
TAVYYCARQNPFYYYDSNYDYAMDFWGQGTTVTVSS
The amino acid sequence of BCMA-mAb01.
VH2a is as shown in SEQ ID NO: 26:
EVQLVESGGGLVQPGGSLRLSCAASGFTFSDFYMYWVRQAPEKRL
EWVAYITNAGENTYFPDTVKGRFTISRDNAKNSLYLQMNSLRAED
TAVYYCARQNPFYYYDSNYDYAMDFWGQGTTVTVSS
The amino acid sequence of BCMA-mAb01.
VH3 is as shown in SEQ ID NO: 27:
EVKLVESGGGLVQPGGSLRLSCAASGFTFSDFYMYWVRQAPEKRL
EWVAYITNGGENTYFPDTVKGRFTISRDNAKNSLYLQMNSLRAED
TAVYYCARQNPFYYYDSNYDYAMDFWGQGTTVTVSS
The amino acid sequence of humanization
light chain template IGKV2-30*01 is as
shown in SEQ ID NO: 28:
DVVMTQSPLSLPVTLGQPASISCRSSQSLVYSDGNTYLNWFQQRP
GQSPRRLIYKVSNRDSGVPDRFSGSGSGTDFTLKISRVEAEDVGV
YYCMQGTHWP
The amino acid sequence of humanization
light chain template IGKJ4*01 is as shown in
SEQ ID NO: 29:
FGGGTKVEIK
The amino acid sequence of humanization
heavy chain template IGHV3-7*01 is as
shown in SEQ ID NO: 30:
EVQLVESGGGLVQPGGSLRLSCAASGFTFSSYWMSWVRQAPGKGL
EWVANIKQDGSEKYYVDSVKGRFTISRDNAKNSLYLQMNSLRAED
TAVYYCAR
The amino acid sequence of humanization
heavy chain template IGHJ6*01 is as shown
in SEQ ID NO: 31:
WGQGTTVTVSS

In the present application, different light chain and heavy chain sequences were selected from the above-mentioned mutation design of the light chain and heavy chain variable regions of the humanized antibody of BCMA-mAb01 for cross combination, respectively, and finally 8 humanized antibodies of BCMA-mAb01 were obtained, and the specific combinations are shown in Table 16.

TABLE 16
Humanized antibody combination of BCMA-mAb01

| Designation of antibody | VL | VH |
|---|---|---|
| BCMA-01 VL1VH1 | BCMA-mAb01.VL1 | BCMA-mAb01.VH1 |
| BCMA-01 VL1VH2 | BCMA-mAb01.VL1 | BCMA-mAb01.VH2 |
| BCMA-01 VL1VH3 | BCMA-mAb01.VL1 | BCMA-mAb01.VH3 |
| BCMA-01 VL1aVH1 | BCMA-mAb01.VL1a | BCMA-mAb01.VH1 |
| BCMA-01 VL2VH1 | BCMA-mAb01.VL2 | BCMA-mAb01.VH1 |
| BCMA-01 VL2VH2 | BCMA-mAb01.VL2 | BCMA-mAb01.VH2 |
| BCMA-01 VL2VH2a | BCMA-mAb01.VL2 | BCMA-mAb01.VH2a |
| BCMA-01 VL2VH3 | BCMA-mAb01.VL2 | BCMA-mAb01.VH3 |

According to the Kabat numbering system, the analysis results of the VH and VL sequences of the above-mentioned humanized antibodies are shown in Table 17.

TABLE 17
Kabat analysis results of the VH and VL
sequences of the humanized antibodies
of BCMA-mAb01

| Variable region | CDR1 | CDR2 | CDR3 |
|---|---|---|---|
| BCMA-mAb01.VL1 | KSSQSL | LVSELD | VQGTHF |
| BCMA-mAb01.VL2 | LYSNGK | S | PHT |
| | TYLS | (SEQ ID | (SEQ ID |
| | (SEQ ID | NO: 108) | NO: 109) |
| | NO: 107) | | |
| BCMA-mAb01.VL1a | KSSQSL | LVSELD | VQGTHF |
| | LYSNAK | S | PHT |
| | TYLS | (SEQ ID | (SEQ ID |
| | (SEQ ID | NO: 108) | NO: 109) |
| | NO: 178) | | |
| BCMA-mAb01.VH1 | DFYMY | YITNGG | QNPFYY |
| BCMA-mAb01.VH2 | (SEQ ID | ENTYFP | YDSNYD |
| BCMA-mAb01.VH3 | NO: 101) | DTVKG | YAMDF |
| | | (SEQ ID | (SEQ ID |
| | | NO: 102) | NO: 103) |
| BCMA-mAb01.VH2a | DFYMY | YITNAG | QNPFYY |
| | (SEQ ID | ENTYFP | YDSNYD |
| | NO: 101) | DTVKG | YAMDF |
| | | (SEQ ID | (SEQ ID |
| | | NO: 179) | NO: 103) |

### (B) Humanization of BCMA-mAb02 Antibody

The specific method was the same as 7 (A). The humanization light chain templates of the murine antibody BCMA-mAb02 were IGKV1-12*01/IGKV4-1*01 and IGKJ2*01, and the humanization heavy chain templates were IGHV3-30*15 and IGHJ6*01. The CDRs of the murine antibody BCMA-mAb02 were grafted into the human templates of the murine antibody respectively, and thus the corresponding humanized versions were obtained. The specific mutation designs are shown in Table 18.

TABLE 18
Mutation design of humanized antibody of BCMA-mAb02

| VL | | VH | |
|---|---|---|---|
| L3 | Graft(IGKV1-12*01) + | H8 | Graft(IGHV3-30*15) + |
| | A43S, L46V, T85D | | A24S, S25N, G26A, F27Y, |
| | | | T28E, S30P, W47L, S75T, N77K |
| L4 | Graft(IGKV4-1*01) + | H9 | Graft(IGHV3-30*15) + |
| | P43S, L46V | | A23E, A24S, S25N, G26A, F27Y, |
| | | | T28E, S30P, W47L, S75T, N77K |

Notes:
Graft represents the grafting of the CDRs of the murine antibody into the FR region sequence of the human germline template; A43S represents the mutation of A at position 43 of Graft into S, and so on. The numbering of backmutated amino acids is according to the natural sequence numbering.

The specific sequence of the variable region of the humanized antibody of BCMA-mAb02 is as follows:

The amino acid sequence of BCMA-mAb02.
VL3 is as shown in SEQ ID NO: 32:
DIQMTQSPSSVSASVGDRVTITCKASQNVGTAVAWYQQKPGKSPK
VLIYSASSRYTGVPSRFSGSGSGTDFTLTISSLQPEDFADYYCQQ
YSNYHLTFGQGTKLEIK
The amino acid sequence of BCMA-mAb02.
VL4 is as shown in SEQ ID NO: 33:
DIVMTQSPDSLAVSLGERATINCKASQNVGTAVAWYQQKPGQSPK
VLIYSASSRYTGVPDRFSGSGSGTDFTLTISSLQAEDVAVYYCQQ
YSNYHLTFGQGTKLEIK
The amino acid sequence of BCMA-mAb02.
VH8 is as shown in SEQ ID NO: 34:
EVQLVESGGGVVQPGRSLRLSCASNAYEFPSYDMSWVRQAPGKGL
ELVAAINSDGGITYYPDTMERRFTISRDNTKKTLYLQMSSLRAED
TAVYYCARHGYGGGAYVLDYWGQGTTVTVSS
The amino acid sequence of BCMA-mAb02.
VH9 is as shown in SEQ ID NO: 35:
EVQLVESGGGVVQPGRSLRLSCESNAYEFPSYDMSWVRQAPGKGL
ELVAAINSDGGITYYPDTMERRFTISRDNTKKTLYLQMSSLRAED
TAVYYCARHGYGGGAYVLDYWGQGTTVTVSS
The amino acid sequence of humanization
light chain template IGKV1-12*01 is as
shown in SEQ ID NO: 36:
DIQMTQSPSSVSASVGDRVTITCRASQGISSWLAWYQQKPGKAPK
LLIYAASSLQSGVPSRFSGSGSGTDFTLTISSLQPEDFATYYCQQ
ANSFP
The amino acid sequence of humanization
light chain template IGKV4-1*01 is as shown
in SEQ ID NO: 37:
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTP
The amino acid sequence of humanization
light chain template IGKJ2*01 is as shown in
SEQ ID NO: 38:
FGQGTKLEIK
The amino acid sequence of humanization
heavy chain template IGHV3-30*15 is as
shown in SEQ ID NO: 39:
QVQLVESGGGVVQPGRSLRLSCAASGFTFSSYAMHWVRQAPGKGL
EWVAVISYDGSNKYYADSVKGRFTISRDNSKNTLYLQMSSLRAED
TAVYYCAR
The amino acid sequence of humanization
heavy chain template IGHJ6*01 is as shown
in SEQ ID NO: 40:
WGQGTTVTVSS

In the present application, different light chain and heavy chain sequences were selected from the above-mentioned mutation design of the light chain and heavy chain variable regions of the humanized antibody of BCMA-mAb02 for cross combination, respectively, and finally 4 humanized antibodies of BCMA-mAb02 were obtained, and the specific combinations are shown in Table 19.

TABLE 19
Humanized antibody combination of BCMA-mAb02

| Designation of antibody | VL | VH |
|---|---|---|
| BCMA-02 VL3VH8 | BCMA-mAb02.VL3 | BCMA-mAb02.VH8 |
| BCMA-02 VL3VH9 | BCMA-mAb02.VL3 | BCMA-mAb02.VH9 |
| BCMA-02 VL4VH8 | BCMA-mAb02.VL4 | BCMA-mAb02.VH8 |
| BCMA-02 VL4VH9 | BCMA-mAb02.VL4 | BCMA-mAb02.VH9 |

According to the Kabat numbering system, the analysis results of the VH and VL sequences of the above-mentioned humanized antibodies are shown in Table 20.

TABLE 20
Kabat analysis results of the VH and VL
sequences of the humanized antibodies
of BCMA-mAb02

| Variable region | CDR1 | CDR2 | CDR3 |
|---|---|---|---|
| BCMA-mAb02.VL3 | KASQNV | SASSRY | QQYSNY |
| BCMA-mAb02.VL4 | GTAVA | T | HLT |
| | (SEQ ID | (SEQ ID | (SEQ ID |
| | NO: 118) | NO: 119) | NO: 120) |
| BCMA-mAb02.VH8 | SYDMS | AINSDG | HGYGGG |
| BCMA-mAb02.VH9 | (SEQ ID | GITYYP | AYVLDY |
| | NO: 112) | DTMER | (SEQ ID |
| | | (SEQ ID | NO: 114) |
| | | NO: 113) | |

### (C) Humanization of BCMA-mAb03 Antibody

The specific method was the same as 7 (A). The humanization light chain templates of the murine antibody BCMA-mAb03 were IGKV1-39*01 and IGKJ4*01, and the humanization heavy chain templates were IGHV7-4-1*02 and IGHJ6*01. The CDRs of the murine antibody BCMA-mAb03 were grafted into the human templates of the murine antibody respectively, and thus the corresponding humanized versions were obtained. The specific mutation designs are shown in Table 21.

TABLE 21
Mutation design of humanized antibody of BCMA-mAb03

| VL | | VH | |
|---|---|---|---|
| L1 | Graft(IGKV1-39*01) + | H1 | Graft(IGHV7-4-1*02) |
| | P44V | | |
| L2 | Graft(IGKV1-39*01) + | H2 | Graft(IGHV7-4-1*02) + D73E, |
| | G41D, A43T, P44V | | V76A |
| L3 | Graft(IGKV1-39*01) + | H3 | Graft(IGHV7-4-1*02) + Q3P, |
| | G41D, K42G, A43T, P44V | | D73E, V76A |
| | | H3a | Graft(IGHV7-4-1*02) + Q3P, |
| | | | D73E, V76A + D63S |

Notes:
Graft represents the grafting of the CDRs of the murine antibody into the FR region sequence of the human germline template; P44V represents the mutation of P at position 44 of Graft into V, and so on. The numbering of backmutated amino acids is according to the natural sequence numbering.

The specific sequence of the variable region of the humanized antibody of BCMA-mAb03 is as follows:

The amino acid sequence of BCMA-mAb03.
VL1 is as shown in SEQ ID NO: 41:
DIQMTQSPSSLSASVGDRVTITCSASQGISNHLNWYQQKPGKAVK
LLIYYTSSLHSGVPSRFSGSGSGTDFTLTISSLQPEDFATYYCQQ
YNNLPWTFGGGTKVEIK
The amino acid sequence of BCMA-mAb03.
VL2 is as shown in SEQ ID NO: 42:
DIQMTQSPSSLSASVGDRVTITCSASQGISNHLNWYQQKPDKTVK
LLIYYTSSLHSGVPSRFSGSGSGTDFTLTISSLQPEDFATYYCQQ
YNNLPWTFGGGTKVEIK
The amino acid sequence of BCMA-mAb03.
VL3 is as shown in SEQ ID NO: 43:
DIQMTQSPSSLSASVGDRVTITCSASQGISNHLNWYQQKPDGTVK
LLIYYTSSLHSGVPSRFSGSGSGTDFTLTISSLQPEDFATYYCQQ
YNNLPWTFGGGTKVEIK
The amino acid sequence of BCMA-mAb03.
VH1 is as shown in SEQ ID NO: 44:
EVQLVQSGSELKKPGASVKVSCKASGYTFTNYAMTWVRQAPGQGL
EWMGWIDTYSGVPTYADDFKGRFVFSLDTSVSTAYLQISSLKAED
TAVYYCARVGVYGGYDTLDSWGQGTTVTVSS
The amino acid sequence of BCMA-mAb03.
VH2 is as shown in SEQ ID NO: 45:
EVQLVQSGSELKKPGASVKVSCKASGYTFTNYAMTWVRQAPGQGL
EWMGWIDTYSGVPTYADDFKGRFVFSLETSASTAYLQISSLKAED
TAVYYCARVGVYGGYDTLDSWGQGTTVTVSS
The amino acid sequence of BCMA-mAb03.
VH3 is as shown in SEQ ID NO: 46:
EVPLVQSGSELKKPGASVKVSCKASGYTFTNYAMTWVRQAPGQGL
EWMGWIDTYSGVPTYADDFKGRFVFSLETSASTAYLQISSLKAED
TAVYYCARVGVYGGYDTLDSWGQGTTVTVSS
The amino acid sequence of BCMA-mAb03.
VH3a is as shown in SEQ ID NO: 47:
EVPLVQSGSELKKPGASVKVSCKASGYTFTNYAMTWVRQAPGQGL
EWMGWIDTYSGVPTYADSFKGRFVFSLETSASTAYLQISSLKAED
TAVYYCARVGVYGGYDTLDSWGQGTTVTVSS
The amino acid sequence of humanization
light chain template IGKV1-39*01 is as
shown in SEQ ID NO: 48:
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTP
The amino acid sequence of humanization
light chain template IGKJ4*01 is as shown in
SEQ ID NO: 49:
FGGGTKVEIK
The amino acid sequence of humanization
heavy chain template IGHV7-4-1*02 is as
shown in SEQ ID NO: 50:
QVQLVQSGSELKKPGASVKVSCKASGYTFTSYAMNWVRQAPGQGL
EWMGWINTNTGNPTYAQGFTGRFVFSLDTSVSTAYLQISSLKAED
TAVYYCAR
The amino acid sequence of humanization
heavy chain template IGHJ6*01 is as shown
in SEQ ID NO: 51: WGQGTTVTVSS

In the present application, different light chain and heavy chain sequences were selected from the above-mentioned mutation design of the light chain and heavy chain variable regions of the humanized antibody of BCMA-mAb03 for cross combination, respectively, and finally 12 humanized antibodies of BCMA-mAb03 were obtained, and the specific combinations are shown in Table 22.

TABLE 22
Humanized antibody combination of BCMA-mAb03

| Designation | | |
|---|---|---|
| of antibody | VL | VH |
| BCMA-03 VL1VH1 | BCMA-F1-mab03.VL1 | BCMA-F1-mab03.VH1 |
| BCMA-03 VL1VH2 | BCMA-F1-mab03.VL1 | BCMA-F1-mab03.VH2 |
| BCMA-03 VL1VH3 | BCMA-F1-mab03.VL1 | BCMA-F1-mab03.VH3 |
| BCMA-03 | BCMA-F1-mab03.VL1 | BCMA-F1- |
| VL1VH3a | | mab03.VH3a |
| BCMA-03 VL2VH1 | BCMA-F1-mab03.VL2 | BCMA-F1-mab03.VH1 |
| BCMA-03 VL2VH2 | BCMA-F1-mab03.VL2 | BCMA-F1-mab03.VH2 |
| BCMA-03 VL2VH3 | BCMA-F1-mab03.VL2 | BCMA-F1-mab03.VH3 |
| BCMA-03 | BCMA-F1-mab03.VL2 | BCMA-F1- |
| VL2VH3a | | mab03.VH3a |
| BCMA-03 VL3VH1 | BCMA-F1-mab03.VL3 | BCMA-F1-mab03.VH1 |
| BCMA-03 VL3VH2 | BCMA-F1-mab03.VL3 | BCMA-F1-mab03.VH2 |
| BCMA-03 VL3VH3 | BCMA-F1-mab03.VL3 | BCMA-F1-mab03.VH3 |
| BCMA-03 | BCMA-F1-mab03.VL3 | BCMA-F1- |
| VL3VH3a | | mab03.VH3a |

According to the Kabat numbering system, the analysis results of the VH and VL sequences of the above-mentioned humanized antibodies are shown in Table 23.

TABLE 23
Kabat analysis results of the
VH and VL sequences of the
humanized antibodies of BCMA-mAb03

| Variable region | CDR1 | CDR2 | CDR3 |
|---|---|---|---|
| BCMA-mAb03.VL1 | SASQGI | YTSSL | QQYNN |
| BCMA-mAb03.VL2 | SNHLN | HS | LPWT |
| BCMA-mAb03.VL3 | (SEQ | (SEQ | (SEQ |
| | ID | ID | ID |
| | NO: | NO: | NO: |
| | 129) | 130) | 131) |
| BCMA-mAb03.VH1 | NYAMT | WIDTY | VGVYG |
| BCMA-mAb03.VH2 | (SEQ | SGVPT | GYDTL |
| BCMA-mAb03.VH3 | ID | YADDF | DS |
| | NO: | KG | (SEQ |
| | 123) | 124) | ID |
| | | | NO: |
| | | | 125) |
| BCMA-mAb03.VH3a | NYAMT | WIDTY | VGVYG |
| | (SEQ | SGVPT | GYDTL |
| | ID | YADSF | DS |
| | NO: | KG | (SEQ |
| | 123) | (SEQ | ID |
| | | ID | NO: |
| | | NO: | 125) |
| | | 180) | |

### (D) Humanization of BCMA-mAb04 Antibody

The specific method was the same as 7 (A). The humanization light chain templates of the murine antibody BCMA-mAb04 were IGKV4-1*01/IGKV1-39*01 and IGKJ4*01, and the humanization heavy chain templates were IGHV1-69*04 and IGHJ6*01. The CDRs of the murine antibody BCMA-mAb04 were grafted into the human templates of the murine antibody respectively, and thus the corresponding humanized versions were obtained. The specific mutation designs are shown in Table 24.

TABLE 24
Mutation design of humanized antibody of BCMA-mAb04

| VL | | VH | |
|---|---|---|---|
| L1 | Graft(IGKV4-1*01) + | H1 | Graft(IGHV1-69*04) + G27Y |
| | P49S | | |
| L2 | Graft(IGKV1-39*01) + | H2 | Graft(IGHV1-69*04) + G27Y, |
| | A49S | | T28S, S30T |
| | | H3 | Graft(IGHV1-69*04) + G27Y, |
| | | | T28S, S30T, I70L |
| | | H3a | Graft(IGHV1-69*04) + G27Y, |
| | | | T28S, S30T, I70L + D56Q |
| | | H4 | Graft(IGHV1-69*04) + V5Q, |
| | | | G27Y, T28S, S30T |
| | | H5 | Graft(IGHV1-69*04) + G27Y, |
| | | | T28S, S30T, Q39L |

Notes:
Graft represents the grafting of the CDRs of the murine antibody into the FR region sequence of the human germline template; P49S represents the mutation of P at position 49 of Graft into S, and so on. The numbering of backmutated amino acids is according to the natural sequence numbering.

The specific sequence of the variable region of the humanized antibody of BCMA-mAb04 is as follows:

The amino acid sequence of BCMA-mAb04.
VL1 is as shown in SEQ ID NO: 52:
DIVMTQSPDSLAVSLGERATINCKSSQSLLYSSNQKNYLAWYQQK
PGQSPKLLIYWASTRYSGVPDRFSGSGSGTDFTLTISSLQAEDVA
VYYCQQYYIYPWTFGGGTKVEIK
The amino acid sequence of BCMA-mAb04.
VL2 is as shown in SEQ ID NO: 53:
DIQMTQSPSSLSASVGDRVTITCKSSQSLLYSSNQKNYLAWYQQK
PGKSPKLLIYWASTRYSGVPSRFSGSGSGTDFTLTISSLQPEDFA
TYYCQQYYIYPWTFGGGTKVEIK
The amino acid sequence of BCMA-mAb04.
VH1 is as shown in SEQ ID NO: 54:
EVQLVQSGAEVKKPGSSVKVSCKASGYTFSSYGISWVRQAPGQGL
EWMGEIYPRDDITYYNEKFKGRVTITADKSTSTAYMELSSLRSED
TAVYYCARHDYYYVNSVSAMDYWGQGTTVTVSS
The amino acid sequence of BCMA-mAb04.
VH2 is as shown in SEQ ID NO: 55:
EVQLVQSGAEVKKPGSSVKVSCKASGYSFTSYGISWVRQAPGQGL
EWMGEIYPRDDITYYNEKFKGRVTITADKSTSTAYMELSSLRSED
TAVYYCARHDYYYVNSVSAMDYWGQGTTVTVSS
The amino acid sequence of BCMA-mAb04.
VH3 is as shown in SEQ ID NO: 56:
EVQLVQSGAEVKKPGSSVKVSCKASGYSFTSYGISWVRQAPGQGL
EWMGEIYPRDDITYYNEKFKGRVTLTADKSTSTAYMELSSLRSED
TAVYYCARHDYYYVNSVSAMDYWGQGTTVTVSS
The amino acid sequence of BCMA-mAb04.
VH3a is as shown in SEQ ID NO: 57:
EVQLVQSGAEVKKPGSSVKVSCKASGYSFTSYGISWVRQAPGQGL
EWMGEIYPRDQITYYNEKFKGRVTLTADKSTSTAYMELSSLRSED
TAVYYCARHDYYYVNSVSAMDYWGQGTTVTVSS
The amino acid sequence of BCMA-mAb04.
VH4 is as shown in SEQ ID NO: 58:
EVQLQQSGAEVKKPGSSVKVSCKASGYSFTSYGISWVRQAPGQGL
EWMGEIYPRDDITYYNEKFKGRVTITADKSTSTAYMELSSLRSED
TAVYYCARHDYYYVNSVSAMDYWGQGTTVTVSS
The amino acid sequence of BCMA-mAb04.
VH5 is as shown in SEQ ID NO: 59:
EVQLVQSGAEVKKPGSSVKVSCKASGYSFTSYGISWVRLAPGQGL
EWMGEIYPRDDITYYNEKFKGRVTITADKSTSTAYMELSSLRSED
TAVYYCARHDYYYVNSVSAMDYWGQGTTVTVSS
The amino acid sequence of humanization
light chain template IGKV4-1*01 is as shown
in SEQ ID NO: 60:
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTP
The amino acid sequence of humanization
light chain template IGKV1-39*01 is as
shown in SEQ ID NO: 61:
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTP
The amino acid sequence of humanization
light chain template IGKJ4*01 is as shown in
SEQ ID NO: 62:
FGGGTKVEIK
The amino acid sequence of humanization
heavy chain template IGHV1-69*04 is as
shown in SEQ ID NO: 63:
QVQLVQSGAEVKKPGSSVKVSCKASGGTFSSYAISWVRQAPGQGL
EWMGRIIPILGIANYAQKFQGRVTITADKSTSTAYMELSSLRSED
TAVYYCAR
The amino acid sequence of humanization
heavy chain template IGHJ6*01 is as shown
in SEQ ID NO: 64:
WGQGTTVTVSS

In the present application, different light chain and heavy chain sequences were selected from the above-mentioned mutation design of the light chain and heavy chain variable regions of the humanized antibody of BCMA-mAb04 for cross combination, respectively, and finally 12 humanized antibodies of BCMA-mAb04 were obtained, and the specific combinations are shown in Table 25.

TABLE 25
Humanized antibody combination of BCMA-mAb04

| Designation of antibody | VL | VH |
|---|---|---|
| BCMA-04 VL1VH1 | BCMA-mAb04.VL1 | BCMA-mAb04.VH1 |
| BCMA-04 VL1VH2 | BCMA-mAb04.VL1 | BCMA-mAb04.VH2 |
| BCMA-04 VL1VH3 | BCMA-mAb04.VL1 | BCMA-mAb04.VH3 |
| BCMA-04 VL1VH3a | BCMA-mAb04.VL1 | BCMA-mAb04.VH3a |
| BCMA-04 VL1VH4 | BCMA-mAb04.VL1 | BCMA-mAb04.VH4 |
| BCMA-04 VL1VH5 | BCMA-mAb04.VL1 | BCMA-mAb04.VH5 |
| BCMA-04 VL2VH1 | BCMA-mAb04.VL2 | BCMA-mAb04.VH1 |
| BCMA-04 VL2VH2 | BCMA-mAb04.VL2 | BCMA-mAb04.VH2 |
| BCMA-04 VL2VH3 | BCMA-mAb04.VL2 | BCMA-mAb04.VH3 |
| BCMA-04 VL2VH3a | BCMA-mAb04.VL2 | BCMA-mAb04.VH3a |
| BCMA-04 VL2VH4 | BCMA-mAb04.VL2 | BCMA-mAb04.VH4 |
| BCMA-04 VL2VH5 | BCMA-mAb04.VL2 | BCMA-mAb04.VH5 |

According to the Kabat numbering system, the analysis results of the VH and VL sequences of the above-mentioned humanized antibodies are shown in Table 26.

TABLE 26
Kabat analysis results of the VH and VL
sequences of the humanized antibodies
of BCMA-mAb04

| Variable region | CDR1 | CDR2 | CDR3 |
|---|---|---|---|
| BCMA-mAb04.VL1 | KSSQS | WAST | QQYYI |
| BCMA-mAb04.VL2 | LLYSS | RYS | YPWT |
| | NQKNY | (SEQ ID | (SEQ ID |
| | LA | NO: 141) | NO: 142) |
| | (SEQ ID | | |
| | NO: 140) | | |
| BCMA-mAb04.VH1 | SYGIS | EIYP | HDYY |
| BCMA-mAb04.VH2 | (SEQ ID | RDDI | YVNS |
| BCMA-mAb04.VH3 | NO: 134) | TYYN | VSAMDY |
| BCMA-mAb04.VH4 | | EKFK | (SEQ ID |
| BCMA-mAb04.VH5 | | G | NO: 136) |
| | | (SEQ ID | |
| | | NO: 135) | |
| BCMA-mAb04. | SYGIS | EIYP | HDYY |
| VH3a | (SEQ ID | RDQI | YVNS |
| | NO: 134) | TYYN | VSAM |
| | | EKFK | DY |
| | | G | (SEQ ID |
| | | (SEQ ID | NO: 136) |
| | | NO: 181) | |

### (E) Humanization of BCMA-mAb05

The specific method was the same as 7 (A). The humanization light chain templates of the murine antibody BCMA-mAb05 were IGKV1-33*01/IGKV4-1*01 and IGKJ4*01, and the humanization heavy chain templates were IGHV1-69*02 and IGHJ6*01. The CDRs of the murine antibody BCMA-mAb05 were grafted into the human templates of the murine antibody respectively, and thus the corresponding humanized versions were obtained. The specific mutation designs are shown in Table 27.

TABLE 27
Mutation design of humanized antibody of BCMA-mAb05

| VL | | VH | |
|---|---|---|---|
| L1 | Graft(IGKV1-33*01) + | H1 | Graft(IGHV1-69*02) + G27Y, |
| | A43S, K45R | | T28M, S30T |
| L2 | Graft(IGKV1-33*01) + | H2 | Graft(IGHV1-69*02) + V2G, |
| | A43S, K45R, S60D | | L4M, G27Y, T28M, S30T |
| L3 | Graft(IGKV4-1*01) + | H2a | Graft(IGHV1-69*02) + V2G, |
| | P43S, K45R | | L4M, G27Y, T28M, S30T + D56Q |
| | | H3 | Graft(IGHV1-69*02) + G27Y, |
| | | | T28M, S30T, V37M |
| | | H4 | Graft(IGHV1-69*02) + G27Y, |
| | | | T28M, S30T, I70L |

Notes:
Graft represents the grafting of the CDRs of the murine antibody into the FR region sequence of the human germline template; A43S represents the mutation of A at position 43 of Graft into S, and so on. The numbering of backmutated amino acids is according to the natural sequence numbering.

The specific sequence of the variable region of the humanized antibody of BCMA-mAb05 is as follows:

The amino acid sequence of BCMA-mAb05.
VL1 is as shown in SEQ ID NO: 65:
DIQMTQSPSSLSASVGDRVTITCKASQDVSTAVAWYQQKPGKSPR
LLIYSASSRYTGVPSRFSGSGSGTDFTFTISSLQPEDIATYYCQQ
LYSTPWTFGGGTKVEIK
The amino acid sequence of BCMA-mAb05.
VL2 is as shown in SEQ ID NO: 66:
DIQMTQSPSSLSASVGDRVTITCKASQDVSTAVAWYQQKPGKSPR
LLIYSASSRYTGVPDRFSGSGSGTDFTFTISSLQPEDIATYYCQQ
LYSTPWTFGGGTKVEIK
The amino acid sequence of BCMA-mAb05.
VL3 is as shown in SEQ ID NO: 67:
DIVMTQSPDSLAVSLGERATINCKASQDVSTAVAWYQQKPGQSPR
LLIYSASSRYTGVPDRFSGSGSGTDFTLTISSLQAEDVAVYYCQQ
LYSTPWTFGGGTKVEIK
The amino acid sequence of BCMA-mAb05.
VH1 is as shown in SEQ ID NO: 68:
EVQLVQSGAEVKKPGSSVKVSCKASGYMFTDHTFHWVRQAPGQGL
EWMGYIYPRDDNTKYNEKFKGRVTITADKSTSTAYMELSSLRSED
TAVYYCARAGYYGSSHAMDYWGQGTTVTVSS
The amino acid sequence of BCMA-mAb05.
VH2 is as shown in SEQ ID NO: 69:
EGQMVQSGAEVKKPGSSVKVSCKASGYMFTDHTFHWVRQAPGQGL
EWMGYIYPRDDNTKYNEKFKGRVTITADKSTSTAYMELSSLRSED
TAVYYCARAGYYGSSHAMDYWGQGTTVTVSS
The amino acid sequence of BCMA-mAb05.
VH2a is as shown in SEQ ID NO: 70:
EGQMVQSGAEVKKPGSSVKVSCKASGYMFTDHTFHWVRQAPGQGL
EWMGYIYPRDQNTKYNEKFKGRVTITADKSTSTAYMELSSLRSED
TAVYYCARAGYYGSSHAMDYWGQGTTVTVSS
The amino acid sequence of BCMA-mAb05.
VH3 is as shown in SEQ ID NO: 71:
EVQLVQSGAEVKKPGSSVKVSCKASGYMFTDHTFHWMRQAPGQGL
EWMGYIYPRDDNTKYNEKFKGRVTITADKSTSTAYMELSSLRSED
TAVYYCARAGYYGSSHAMDYWGQGTTVTVSS
The amino acid sequence of BCMA-mAb05.
VH4 is as shown in SEQ ID NO: 72:
EVQLVQSGAEVKKPGSSVKVSCKASGYMFTDHTFHWVRQAPGQGL
EWMGYIYPRDDNTKYNEKFKGRVTLTADKSTSTAYMELSSLRSED
TAVYYCARAGYYGSSHAMDYWGQGTTVTVSS
The amino acid sequence of humanization
light chain template IGKV1-33*01 is as
shown in SEQ ID NO: 73:
DIQMTQSPSSLSASVGDRVTITCQASQDISNYLNWYQQKPGKAPK
LLIYDASNLETGVPSRFSGSGSGTDFTFTISSLQPEDIATYYCQQ
YDNLP
The amino acid sequence of humanization
light chain template IGKV4-1*01 is as shown
in SEQ ID NO: 74:
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTP
The amino acid sequence of humanization
light chain template IGKJ4*01 is as shown in
SEQ ID NO: 75:
FGGGTKVEIK
The amino acid sequence of humanization
heavy chain template IGHV1-69*02 is as
shown in SEQ ID NO: 76:
QVQLVQSGAEVKKPGSSVKVSCKASGGTFSSYTISWVRQAPGQGL
EWMGRIPILGIANYAQKFQGRVTITADKSTSTAYMELSSLRSEDT
AVYYCAR
The amino acid sequence of humanization
heavy chain template IGHJ6*01 is as shown
in SEQ ID NO: 77:
WGQGTTVTVSS

In the present application, different light chain and heavy chain sequences were selected from the above-mentioned mutation design of the light chain and heavy chain variable regions of the humanized antibody of BCMA-mAb05 for cross combination, respectively, and finally 15 humanized antibodies of BCMA-mAb05 were obtained, and the specific combinations are shown in Table 28.

TABLE 28
Humanized antibody combination of BCMA-mAb05

| Designation of antibody | VL | VH |
|---|---|---|
| BCMA-05 VL1VH1 | BCMA-mAb05.VL1 | BCMA-mAb05.VH1 |
| BCMA-05 VL1VH2 | BCMA-mAb05.VL1 | BCMA-mAb05.VH2 |
| BCMA-05 VL1VH2a | BCMA-mAb05.VL1 | BCMA-mAb05.VH2a |
| BCMA-05 VL1VH3 | BCMA-mAb05.VL1 | BCMA-mAb05.VH3 |
| BCMA-05 VL1VH4 | BCMA-mAb05.VL1 | BCMA-mAb05.VH4 |
| BCMA-05 VL2VH1 | BCMA-mAb05.VL2 | BCMA-mAb05.VH1 |
| BCMA-05 VL2VH2 | BCMA-mAb05.VL2 | BCMA-mAb05.VH2 |
| BCMA-05 VL2VH2a | BCMA-mAb05.VL2 | BCMA-mAb05.VH2a |
| BCMA-05 VL2VH3 | BCMA-mAb05.VL2 | BCMA-mAb05.VH3 |
| BCMA-05 VL2VH4 | BCMA-mAb05.VL2 | BCMA-mAb05.VH4 |
| BCMA-05 VL3VH1 | BCMA-mAb05.VL3 | BCMA-mAb05.VH1 |
| BCMA-05 VL3VH2 | BCMA-mAb05.VL3 | BCMA-mAb05.VH2 |
| BCMA-05 VL3VH2a | BCMA-mAb05.VL3 | BCMA-mAb05.VH2a |
| BCMA-05 VL3VH3 | BCMA-mAb05.VL3 | BCMA-mAb05.VH3 |
| BCMA-05 VL3VH4 | BCMA-mAb05.VL3 | BCMA-mAb05.VH4 |

According to the Kabat numbering system, the analysis results of the VH and VL sequences of the above-mentioned humanized antibodies are shown in Table 29.

TABLE 29
Kabat analysis results of the VH and VL
sequences of the humanized antibodies
of BCMA-mAb05

| Variable region | CDR1 | CDR2 | CDR3 |
|---|---|---|---|
| BCMA-mAb05.VL1 | KASQD | SASS | QQLY |
| BCMA-mAb05.VL2 | VSTAV | RYT | STPW |
| BCMA-mAb05.VL3 | A | (SEQ | T |
| | (SEQ | ID | (SEQ |
| | ID | NO: | ID |
| | NO: | 152) | NO: |
| | 151) | | 153) |
| BCMA-mAb05.VH1 | DHTFH | YIYP | AGYY |
| BCMA-mAb05.VH2 | (SEQ | RDDN | GSSH |
| BCMA-mAb05.VH3 | ID | TKYN | AMDY |
| BCMA-mAb05.VH4 | NO: | EKFK | (SEQ |
| | 145) | G | ID |
| | | (SEQ | NO: |
| | | ID | 147) |
| | | NO: | |
| | | 146) | |
| BCMA-mAb05.VH2a | DHTFH | YIYP | AGYY |
| | (SEQ | RDQN | GSSH |
| | ID | TKYN | AMDY |
| | NO: | EKFK | (SEQ |
| | 145) | G | ID |
| | | (SEQ | NO: |
| | | ID | 147) |
| | | NO: | |
| | | 182) | |

### (F) Humanization of BCMA-mAb06

The specific method was the same as 7 (A). The humanization light chain templates of the murine antibody BCMA-mAb06 were IGKV1-12*01/IGKV4-1*01 and IGKJ2*01, and the humanization heavy chain templates were IGHV3-7*01 and IGHJ6*01. The CDRs of the murine antibody BCMA-mAb06 were grafted into the human templates of the murine antibody respectively, and thus the corresponding humanized versions were obtained. The specific mutation designs are shown in Table 30.

TABLE 30
Mutation design of humanized antibody of BCMA-mAb06

| VL | | VH | |
|---|---|---|---|
| L1 | Graft(IGKV1-12*01) + | H1 | Graft(IGHV3-7*01) + F27I, |
| | A43S, L46A | | T28D, R98I |
| L1a | Graft(IGKV1-12*01) + | H2 | Graft(IGHV3-7*01) + Q3K, |
| | A43S, L46A + N92Q | | F27I, T28D, R98I |
| L2 | Graft(IGKV4-1*01) + | H3 | Graft(IGHV3-7*01) + Q3K, |
| | P43S, L46A | | V5L, E6Q, F27I, T28D, R98I |
| L2a | Graft(IGKV4-1*01) + | | |
| | P43S, L46A + G93A | | |

Notes:
Graft represents the grafting of the CDRs of the murine antibody into the FR region sequence of the human germline template; A43S represents the mutation of A at position 43 of Graft into S, and so on. The numbering of backmutated amino acids is according to the natural sequence numbering.

The specific sequence of the variable region of the humanized antibody of BCMA-mAb06 is as follows:

The amino acid sequence of BCMA-mAb06.
VL1 is as shown in SEQ ID NO: 78:
DIQMTQSPSSVSASVGDRVTITCKASQNVDTNVAWYQQKPGKSPK
ALIYSASYRFSGVPSRFSGSGSGTDFTLTISSLQPEDFATYYCQQ
YNGYPFTFGQGTKLEIK
The amino acid sequence of BCMA-mAb06.
VL1a is as shown in SEQ ID NO: 79:
DIQMTQSPSSVSASVGDRVTITCKASQNVDTNVAWYQQKPGKSPK
ALIYSASYRFSGVPSRFSGSGSGTDFTLTISSLQPEDFATYYCQQ
YQGYPFTFGQGTKLEIK
The amino acid sequence of BCMA-mAb06.
VL2 is as shown in SEQ ID NO: 80:
DIVMTQSPDSLAVSLGERATINCKASQNVDTNVAWYQQKPGQSPK
ALIYSASYRFSGVPDRFSGSGSGTDFTLTISSLQAEDVAVYYCQQ
YNGYPFTFGQGTKLEIK
The amino acid sequence of BCMA-mAb06.
VL2a is as shown in SEQ ID NO: 81:
DIVMTQSPDSLAVSLGERATINCKASQNVDTNVAWYQQKPGQSPK
ALIYSASYRFSGVPDRFSGSGSGTDFTLTISSLQAEDVAVYYCQQ
YNAYPFTFGQGTKLEIK
The amino acid sequence of BCMA-mAb06.
VH1 is as shown in SEQ ID NO: 82:
EVQLVESGGGLVQPGGSLRLSCAASGIDFSRYWMSWVRQAPGKGL
EWVAEINPDSSTINYAPSLKDRFTISRDNAKNSLYLQMNSLRAED
TAVYYCAIFYYANYYAMDYWGQGTTVTVSS
The amino acid sequence of BCMA-mAb06.
VH2 is as shown in SEQ ID NO: 83:
EVKLVESGGGLVQPGGSLRLSCAASGIDFSRYWMSWVRQAPGKGL
EWVAEINPDSSTINYAPSLKDRFTISRDNAKNSLYLQMNSLRAED
TAVYYCAIFYYANYYAMDYWGQGTTVTVSS
The amino acid sequence of BCMA-mAb06.
VH3 is as shown in SEQ ID NO: 84:
EVKLLQSGGGLVQPGGSLRLSCAASGIDFSRYWMSWVRQAPGKGL
EWVAEINPDSSTINYAPSLKDRFTISRDNAKNSLYLQMNSLRAED
TAVYYCAIFYYANYYAMDYWGQGTTVTVSS
The amino acid sequence of humanization
light chain template IGKV1-12*01 is as
shown in SEQ ID NO: 85:
DIQMTQSPSSVSASVGDRVTITCRASQGISSWLAWYQQKPGKAPK
LLIYAASSLQSGVPSRFSGSGSGTDFTLTISSLQPEDFATYYCQQ
ANSFP
The amino acid sequence of humanization
light chain template IGKV4-1*01 is as shown
in SEQ ID NO: 86:
DIVMTQSPDSLAVSLGERATINCKSSQSVLYSSNNKNYLAWYQQK
PGQPPKLLIYWASTRESGVPDRFSGSGSGTDFTLTISSLQAEDVA
VYYCQQYYSTP
The amino acid sequence of humanization
light chain template IGKJ2*01 is as shown in
SEQ ID NO: 87:
FGQGTKLEIK
The amino acid sequence of humanization
heavy chain template IGHV3-7*01 is as
shown in SEQ ID NO: 88:
EVQLVESGGGLVQPGGSLRLSCAASGFTFSSYWMSWVRQAPGKGL
EWVANIKQDGSEKYYVDSVKGRFTISRDNAKNSLYLQMNSLRAED
TAVYYCAR
The amino acid sequence of humanization
heavy chain template IGHJ6*01 is as shown
in SEQ ID NO: 89: WGQGTTVTVSS

In the present application, different light chain and heavy chain sequences were selected from the above-mentioned mutation design of the light chain and heavy chain variable regions of the humanized antibody of BCMA-mAb06 for cross combination, respectively, and finally 12 humanized antibodies of BCMA-mAb06 were obtained, and the specific combinations are shown in Table 31.

TABLE 31
Humanized antibody combination of BCMA-mAb06

| Designation of antibody | VL | VH |
|---|---|---|
| BCMA-06 VL1VH1 | BCMA-mAb06.VL1 | BCMA-mAb06.VH1 |
| BCMA-06 VL1VH2 | BCMA-mAb06.VL1 | BCMA-mAb06.VH2 |
| BCMA-06 VL1VH3 | BCMA-mAb06.VL1 | BCMA-mAb06.VH3 |
| BCMA-06 VL1aVH1 | BCMA-mAb06.VL1a | BCMA-mAb06.VH1 |
| BCMA-06 VL1aVH2 | BCMA-mAb06.VL1a | BCMA-mAb06.VH2 |
| BCMA-06 VL1aVH3 | BCMA-mAb06.VL1a | BCMA-mAb06.VH3 |
| BCMA-06 VL2VH1 | BCMA-mAb06.VL2 | BCMA-mAb06.VH1 |
| BCMA-06 VL2VH2 | BCMA-mAb06.VL2 | BCMA-mAb06.VH2 |
| BCMA-06 VL2VH3 | BCMA-mAb06.VL2 | BCMA-mAb06.VH3 |
| BCMA-06 VL2aVH1 | BCMA-mAb06.VL2a | BCMA-mAb06.VH1 |
| BCMA-06 VL2aVH2 | BCMA-mAb06.VL2a | BCMA-mAb06.VH2 |
| BCMA-06 VL2aVH3 | BCMA-mAb06.VL2a | BCMA-mAb06.VH3 |

According to the Kabat numbering system, the analysis results of the VH and VL sequences of the above-mentioned humanized antibodies are shown in Table 32.

TABLE 32
Kabat analysis results of the VH and VL
sequences of the humanized antibodies
of BCMA-mAb06

| Variable region | CDR1 | CDR2 | CDR3 |
|---|---|---|---|
| BCMA-mAb06.VL1 | KASQNV | SASYRF | QQYNGY |
| BCMA-mAb06.VL2 | DTNVA | S | PFT |
| | (SEQ ID | (SEQ ID | (SEQ ID |
| | NO: 162) | NO: 163) | NO: 164) |
| BCMA- | KASQNV | SASYRF | QQYQGY |
| mAb06.VL1a | DTNVA | S | PFT |
| | (SEQ ID | (SEQ ID | (SEQ ID |
| | NO: 162) | NO: 163) | NO: 183) |
| BCMA- | KASQNV | SASYRF | QQYNAY |
| mAb06.VL2a | DTNVA | S | PFT |
| | (SEQ ID | (SEQ ID | (SEQ ID |
| | NO: 162) | NO: 163) | NO: 184) |
| BCMA-mAb06.VH1 | RYWMS | EINPDS | FYYANY |
| BCMA-mAb06.VH2 | (SEQ ID | STINYA | YAMDY |
| BCMA-mAb06.VH3 | NO: 156) | PSLKD | (SEQ ID |
| | | (SEQ ID | NO: 158) |
| | | NO: 157) | |

### (G) Humanization of BCMA-mAb07

The specific method was the same as 7 (A). The humanization light chain templates of the murine antibody BCMA-mAb07 were IGKV1-17*01 and IGKJ2*01, and the humanization heavy chain templates were IGHV1-69*02 and IGHJ6*01. The CDRs of the murine antibody BCMA-mAb07 were grafted into the human templates of the murine antibody respectively, and thus the corresponding humanized versions were obtained. The specific mutation designs are shown in Table 33.

TABLE 33
Mutation design of humanized antibody of BCMA-mAb07

| VL | | VH | |
|---|---|---|---|
| L1 | Graft(IGKV1-17*01) + A42S | H1 | Graft(IGHV1-69*02) + G27Y, |
| | | | S30T |
| L2 | Graft(IGKV1-17*01) + A42S, | H2 | Graft(IGHV1-69*02) + G27Y, |
| | L46W | | S30T, I70L |
| L3 | Graft(IGKV1-17*01) + D1Q, | H3 | Graft(IGHV1-69*02) + G27Y, |
| | A42S, L46W | | S30T, M48I, V68A, I70L |
| L4 | Graft(IGKV1-17*01) + D1Q, | | |
| | K41T, A42S, L46W | | |

Notes:
Graft represents the grafting of the CDRs of the murine antibody into the FR region sequence of the human germline template; A42S represents the mutation of A at position 42 of Graft into S, and so on. The numbering of backmutated amino acids is according to the natural sequence numbering.

The specific sequence of the variable region of the humanized antibody of BCMA-mAb07 is as follows:

The amino acid sequence of BCMA-mAb07.
VL1 is as shown in SEQ ID NO: 90:
DIQMTQSPSSLSASVGDRVTITCSASSSVSYMHWYQQKPGKSPKR
LIYDTSKLASGVPSRFSGSGSGTEFTLTISSLQPEDFATYYCQQW
SSNPLTFGQGTKLEIK
The amino acid sequence of BCMA-mAb07.
VL2 is as shown in SEQ ID NO: 91:
DIQMTQSPSSLSASVGDRVTITCSASSSVSYMHWYQQKPGKSPKR
WIYDTSKLASGVPSRFSGSGSGTEFTLTISSLQPEDFATYYCQQW
SSNPLTFGQGTKLEIK
The amino acid sequence of BCMA-mAb07.
VL3 is as shown in SEQ ID NO: 92:
QIQMTQSPSSLSASVGDRVTITCSASSSVSYMHWYQQKPGKSPKR
WIYDTSKLASGVPSRFSGSGSGTEFTLTISSLQPEDFATYYCQQW
SSNPLTFGQGTKLEIK
The amino acid sequence of BCMA-mAb07.
VL4 is as shown in SEQ ID NO: 93:
QIQMTQSPSSLSASVGDRVTITCSASSSVSYMHWYQQKPGTSPKR
WIYDTSKLASGVPSRFSGSGSGTEFTLTISSLQPEDFATYYCQQW
SSNPLTFGQGTKLEIK
The amino acid sequence of BCMA-mAb07.
VH1 is as shown in SEQ ID NO: 94:
EVQLVQSGAEVKKPGSSVKVSCKASGYTFTDYWTHWVRQAPGQGL
EWMGYINHGSGYTKYNQKFKDRVTITADKSTSTAYMELSSLRSED
TAVYYCARLTGSYYFDYWGQGTTVTVSS
The amino acid sequence of BCMA-mAb07.
VH2 is as shown in SEQ ID NO: 95:
EVQLVQSGAEVKKPGSSVKVSCKASGYTFTDYWTHWVRQAPGQGL
EWMGYINHGSGYTKYNQKFKDRVTLTADKSTSTAYMELSSLRSED
TAVYYCARLTGSYYFDYWGQGTTVTVSS
The amino acid sequence of BCMA-mAb07.
VH3 is as shown in SEQ ID NO: 96:
EVQLVQSGAEVKKPGSSVKVSCKASGYTFTDYWTHWVRQAPGQGL
EWIGYINHGSGYTKYNQKFKDRATLTADKSTSTAYMELSSLRSED
TAVYYCARLTGSYYFDYWGQGTTVTVSS
The amino acid sequence of humanization
light chain template IGKV1-17*01 is as
shown in SEQ ID NO: 97:
DIQMTQSPSSLSASVGDRVTITCRASQGIRNDLGWYQQKPGKAPK
RLIYAASSLQSGVPSRFSGSGSGTEFTLTISSLQPEDFATYYCLQ
HNSYP
The amino acid sequence of humanization
light chain template IGKJ2*01 is as shown in
SEQ ID NO: 98:
FGQGTKLEIK
The amino acid sequence of humanization
heavy chain template IGHV1-69*02 is as
shown in SEQ ID NO: 99:
QVQLVQSGAEVKKPGSSVKVSCKASGGTFSSYTISWVRQAPGQGL
EWMGRIIPILGIANYAQKFQGRVTITADKSTSTAYMELSSLRSED
TAVYYCAR
The amino acid sequence of humanization
heavy chain template IGHJ6*01 is as shown
in SEQ ID NO: 100:
WGQGTTVTVSS

In the present application, different light chain and heavy chain sequences were selected from the above-mentioned mutation design of the light chain and heavy chain variable regions of the humanized antibody of BCMA-mAb07 for cross combination, respectively, and finally 12 humanized antibodies of BCMA-mAb07 were obtained, and the specific combinations are shown in Table 34.

TABLE 34
Humanized antibody combination of BCMA-MAB07

| Designation of antibody | VL | VH |
|---|---|---|
| BCMA-07 VL1VH1 | BCMA-mAb07.VL1 | BCMA-mAb07.VH1 |
| BCMA-07 VL1VH2 | BCMA-mAb07.VL1 | BCMA-mAb07.VH2 |
| BCMA-07 VL1VH3 | BCMA-mAb07.VL1 | BCMA-mAb07.VH3 |
| BCMA-07 VL2VH1 | BCMA-mAb07.VL2 | BCMA-mAb07.VH1 |
| BCMA-07 VL2VH2 | BCMA-mAb07.VL2 | BCMA-mAb07.VH2 |
| BCMA-07 VL2VH3 | BCMA-mAb07.VL2 | BCMA-mAb07.VH3 |
| BCMA-07 VL3VH1 | BCMA-mAb07.VL3 | BCMA-mAb07.VH1 |
| BCMA-07 VL3VH2 | BCMA-mAb07.VL3 | BCMA-mAb07.VH2 |
| BCMA-07 VL3VH3 | BCMA-mAb07.VL3 | BCMA-mAb07.VH3 |
| BCMA-07 VL4VH1 | BCMA-mAb07.VL4 | BCMA-mAb07.VH1 |
| BCMA-07 VL4VH2 | BCMA-mAb07.VL4 | BCMA-mAb07.VH2 |
| BCMA-07 VL4VH3 | BCMA-mAb07.VL4 | BCMA-mAb07.VH3 |

According to the Kabat numbering system, the analysis results of the VH and VL sequences of the above-mentioned humanized antibodies are shown in Table 35.

TABLE 35
Kabat analysis results of the VH and VL
sequences of the humanized antibodies
of BCMA-mAb07

| Variable region | CDR1 | CDR2 | CDR3 |
|---|---|---|---|
| BCMA-mAb07.VL1 | SASSS | DTSKLAS | QQWSS |
| BCMA-mAb07.VL2 | VSYMH | (SEQ ID | NPLT |
| BCMA-mAb07.VL3 | (SEQ ID | NO: 174) | (SEQ ID |
| BCMA-mAb07.VL4 | NO: 173) | | NO: 175) |
| BCMA-mAb07.VH1 | DYWTH | YINHGSGY | LTGSY |
| BCMA-mAb07.VH2 | (SEQ ID | TKYNQKFK | YFDY |
| BCMA-mAb07.VH3 | NO: 167) | D | (SEQ ID |
| | | (SEQ ID | NO: 169) |
| | | NO: 168) | |

### Example 8 Construction, Expression and Purity Determination of Humanized Antibodies

The VH and VL fragments of humanized antibody molecules were linked through a linker composed of 15 flexible amino acids (GGGGSGGGGSGGGGS) to form scFv, and fused to human Fc to construct a humanized scFv-Fc eukaryotic expression vector (for the human Fc sequence, see SEQ ID NO: 6). Expi 293F cells in the logarithmic growth phase were transiently transfected with polyethylenimine (PEI) (purchased from Sigma-Aldrich, catalog number: 408727). The culture supernatant was collected 5-7 days after transfection and purified by Protein A affinity. The purity of the obtained antibody was qualitatively analyzed by SEC-HPLC. The results are shown in Table 36. Some antibodies have not been subjected to SEC-HPLC purity analysis and are indicated as “not tested” in the table. The specific purification and analysis methods were the same as those in Example 4.

TABLE 36
Results of expression quantity and SEC purity of BCMA humanized antibody

| | | Expression | | | SEC |
|---|---|---|---|---|---|
| Designation of | Expression | volume | Concentration | Volume | monomer |
| antibody | system | (ml) | (mg/ml) | (ml) | purity % |
| BCMA-01 VL1VH1 | Expi293F | 40 | 3 | 2 | 95.34 |
| BCMA-01 VL1VH2 | Expi293F | 40 | 0.12 | 2 | 91.72 |
| BCMA-01 VL1VH3 | Expi293F | 40 | 0.26 | 2 | 90.27 |
| BCMA-01 VL1aVH1 | Expi293F | 40 | 0.16 | 2 | 91.57 |
| BCMA-01 VL2VH1 | Expi293F | 40 | 1.92 | 2 | 95.68 |
| BCMA-01 VL2VH2 | Expi293F | 40 | 0.15 | 2 | 90.79 |
| BCMA-01 VL2VH2a | Expi293F | 40 | 0.21 | 2 | 81.18 |
| BCMA-01 VL2VH3 | Expi293F | 40 | 0.23 | 2 | 90.66 |
| BCMA-03 VL1VH1 | Expi293F | 40 | 0.5 | 2 | 82.86 |
| BCMA-03 VL1VH2 | Expi293F | 40 | 0.44 | 2 | 81.82 |
| BCMA-03 VL1VH3 | Expi293F | 40 | 0.25 | 2 | 92.86 |
| BCMA-03 VL1VH3a | Expi293F | 40 | 0.35 | 2 | 84.62 |
| BCMA-03 VL2VH1 | Expi293F | 40 | 0.29 | 2 | 78.1 |
| BCMA-03 VL2VH2 | Expi293F | 40 | 0.18 | 2 | 79.92 |
| BCMA-03 VL2VH3 | Expi293F | 40 | 0.1 | 2 | 84.93 |
| BCMA-03 VL2VH3a | Expi293F | 40 | 0.1 | 2 | 77.53 |
| BCMA-03 VL3VH1 | Expi293F | 40 | 1.92 | 2 | 84.69 |
| BCMA-03 VL3VH2 | Expi293F | 40 | 1.95 | 2 | 89.16 |
| BCMA-03 VL3VH3 | Expi293F | 40 | 2.23 | 2 | 90 |
| BCMA-03 VL3VH3a | Expi293F | 40 | 2.26 | 2 | 87.22 |
| BCMA-04 VL1VH1 | Expi293F | 40 | 0.96 | 2 | 64.86 |
| BCMA-04 VL1VH2 | Expi293F | 40 | 1.26 | 2 | 66.96 |
| BCMA-04 VL1VH3 | Expi293F | 40 | 1.49 | 2 | 58.92 |
| BCMA-04 VL1VH3a | Expi293F | 40 | 1.17 | 2 | 62.43 |
| BCMA-04 VL1VH4 | Expi293F | 40 | 1.22 | 2 | 67.67 |
| BCMA-04 VL1VH5 | Expi293F | 40 | 1.18 | 2 | 72.57 |
| BCMA-04 VL2VH1 | Expi293F | 40 | 2.48 | 2 | 75.98 |
| BCMA-04 VL2VH2 | Expi293F | 40 | 2.15 | 2 | 79.08 |
| BCMA-04 VL2VH3 | Expi293F | 40 | 2.13 | 2 | 71.39 |
| BCMA-04 VL2VH3a | Expi293F | 40 | 1.39 | 2 | 68.67 |
| BCMA-04 VL2VH4 | Expi293F | 40 | 1.43 | 2 | 79.64 |
| BCMA-04 VL2VH5 | Expi293F | 40 | 1.5 | 2 | 73.86 |
| BCMA-02 VL3VH8 | Expi293F | 40 | 1.73 | 2 | 97.9 |
| BCMA-02 VL3VH9 | Expi293F | 40 | 1.97 | 2 | 91.66 |
| BCMA-02 VL4VH8 | Expi293F | 40 | 1.82 | 2 | 98.59 |
| BCMA-02 VL4VH9 | Expi293F | 40 | 1.82 | 2 | 98.62 |
| BCMA-05 VL1VH1 | Expi293F | 40 | 0.84 | 2 | Not tested |
| BCMA-05 VL1VH2 | Expi293F | 40 | 0.96 | 2 | |
| BCMA-05 VL1VH2a | Expi293F | 40 | 0.9 | 2 | 83.95 |
| BCMA-05 VL1VH3 | Expi293F | 40 | 1 | 2 | Not tested |
| BCMA-05 VL1VH4 | Expi293F | 40 | 0.64 | 2 | |
| BCMA-05 VL2VH1 | Expi293F | 40 | 1.05 | 2 | |
| BCMA-05 VL2VH2 | Expi293F | 40 | 0.57 | 2 | |
| BCMA-05 VL2VH2a | Expi293F | 40 | 0.82 | 2 | |
| BCMA-05 VL2VH3 | Expi293F | 40 | 0.98 | 2 | |
| BCMA-05 VL2VH4 | Expi293F | 40 | 0.54 | 2 | |
| BCMA-05 VL3VH1 | Expi293F | 40 | 0.87 | 2 | |
| BCMA-05 VL3VH2 | Expi293F | 40 | 0.76 | 2 | |
| BCMA-05 VL3VH2a | Expi293F | 40 | 0.5 | 2 | |
| BCMA-05 VL3VH3 | Expi293F | 40 | 0.42 | 2 | |
| BCMA-05 VL3VH4 | Expi293F | 40 | 1.53 | 2 | |
| BCMA-06 VL1VH1 | Expi293F | 40 | 4.48 | 2 | |
| BCMA-06 VL1VH2 | Expi293F | 40 | 3.472 | 2 | |
| BCMA-06 VL1VH3 | Expi293F | 40 | 0.955 | 2 | |
| BCMA-06 VL1aVH1 | Expi293F | 40 | 4.415 | 2 | |
| BCMA-06 VL1aVH2 | Expi293F | 40 | 3.827 | 2 | |
| BCMA-06 VL1aVH3 | Expi293F | 40 | 3.937 | 2 | 90.76 |
| BCMA-06 VL2VH1 | Expi293F | 40 | 2.139 | 2 | Not tested |
| BCMA-06 VL2VH2 | Expi293F | 40 | 0.938 | 2 | |
| BCMA-06 VL2VH3 | Expi293F | 40 | 0.694 | 2 | |
| BCMA-06 VL2aVH1 | Expi293F | 40 | 3.776 | 2 | |
| BCMA-06 VL2aVH2 | Expi293F | 40 | 3.186 | 2 | |
| BCMA-06 VL2aVH3 | Expi293F | 40 | 0.451 | 2 | |
| BCMA-07 VL1VH1 | Expi293F | 40 | 1.44 | 2 | |
| BCMA-07 VL1VH2 | Expi293F | 40 | 1.177 | 2 | |
| BCMA-07 VL1VH3 | Expi293F | 40 | 3.537 | 2 | |
| BCMA-07 VL2VH1 | Expi293F | 40 | 1.619 | 2 | |
| BCMA-07 VL2VH2 | Expi293F | 40 | 1.977 | 2 | 90.76 |
| BCMA-07 VL2VH3 | Expi293F | 40 | 2.22 | 2 | Not tested |
| BCMA-07 VL3VH1 | Expi293F | 40 | 1.96 | 2 | 90.1 |
| BCMA-07 VL3VH2 | Expi293F | 40 | 1.72 | 2 | Not tested |
| BCMA-07 VL3VH3 | Expi293F | 40 | 1.036 | 2 | |
| BCMA-07 VL4VH1 | Expi293F | 40 | 0.72 | 2 | |
| BCMA-07 VL4VH2 | Expi293F | 40 | 0.853 | 2 | 79.87 |
| BCMA-07 VL4VH3 | Expi293F | 40 | 0.522 | 2 | Not tested |

### Example 9 Identification of Humanized Antibodies

(a) Enzyme-Linked Immunosorbent Assay (ELISA) Detection of Binding of BCMA Humanized antibodies to human BCMA protein and cross-binding activity of BCMA humanized antibodies to monkey BCMA protein

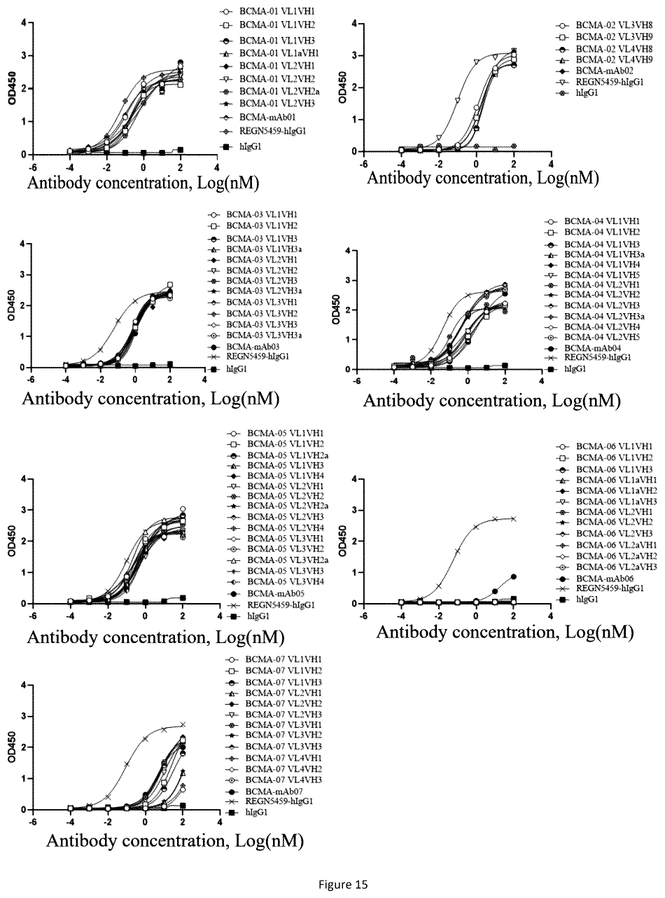

The specific method was the same as that in Example 5 (A). The results are shown in FIG. 14 and FIG. 15. The humanized antibodies bind well to the human BCMA protein. Except for the humanized antibody of BCMA-mAb06, which does not cross-bind to the monkey BCMA protein, the other antibodies all cross-bind to the monkey BCMA protein, the IgG subtype control is human lgG1.

### (B) Flow Cytometry Experiment (FACS) Detection of Binding of Humanized Antibodies to Endogenous Cells Expressing BCMA

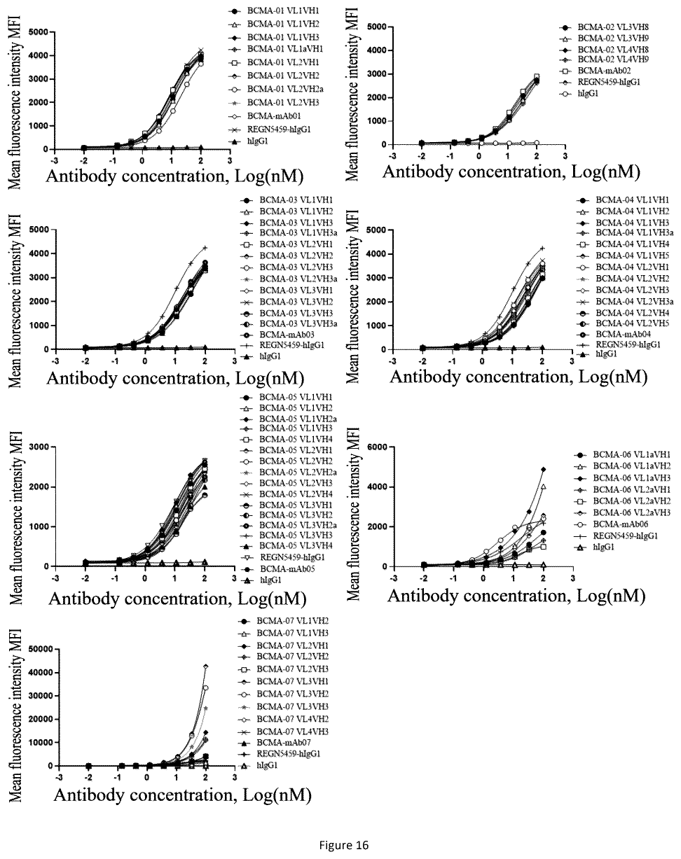

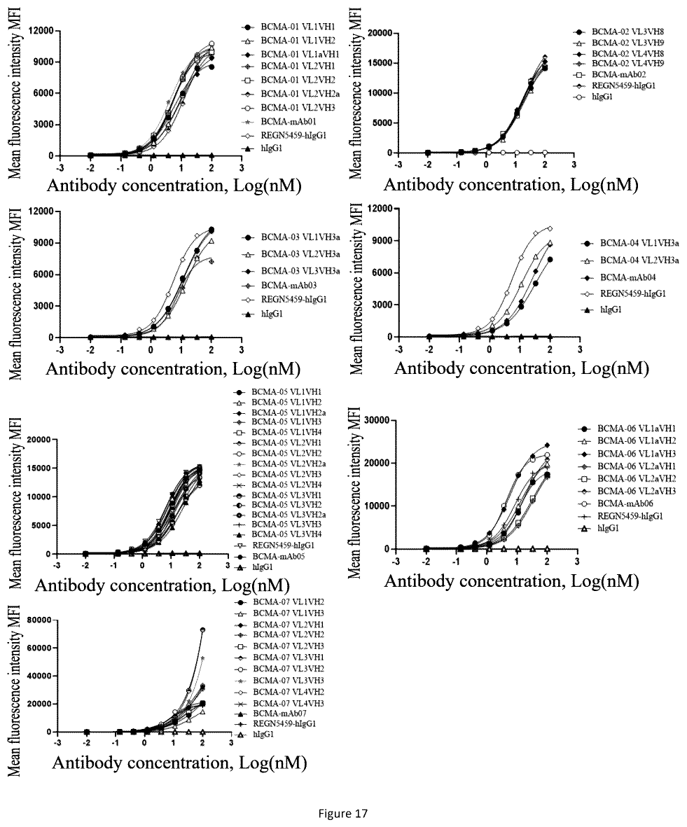

The specific method was the same as that in Example 5 (C). First, the binding of humanized antibodies to U266 cells was detected. Based on the results, some humanized antibodies were selected and tested for binding to H929 cells. The remaining humanized antibodies were not tested for binding to H929 cells by FACS (indicated by “/” in the table). The overall results are shown in Table 37 and FIGS. 16 and 17. All humanized antibodies bind to U266 cells, and all humanized antibodies that bind to the H929 cell in the test bind to H929 cells.

TABLE 37
FACS detection of binding activity of humanized
antibodies with U266 and H929 cells

| | U266 | | H929 | |
|---|---|---|---|---|
| | Maximum | | Maximum | |
| | mean | | mean | |
| Designation | fluorescence | | fluorescence | |
| of antibody | intensity | EC50 (nM) | intensity | EC50 (nM) |
| BCMA-01 VL1VH1 | 3863 | 9.84 | 8532 | 5.58 |
| BCMA-01 VL1VH2 | 3843 | 12.14 | 10373 | 10.96 |
| BCMA-01 VL1VH3 | 3997 | 13.80 | / | / |
| BCMA-01 VL1aVH1 | 3928 | 12.67 | 9374 | 8.70 |
| BCMA-01 VL2VH1 | 4029 | 8.69 | 9562 | 4.96 |
| BCMA-01 VL2VH2 | 3990 | 7.84 | 9984 | 5.22 |
| BCMA-01 VL2VH2a | 3649 | 20.09 | 9715 | 11.78 |
| BCMA-01 VL2VH3 | 4095 | 10.52 | 10779 | 6.13 |
| BCMA-mAb01 | 3934 | 7.48 | 10527 | 4.12 |
| BCMA-03 VL1VH1 | 3372 | 46.51 | / | / |
| BCMA-03 VL1VH2 | 3298 | 30.27 | / | / |
| BCMA-03 VL1VH3 | 3501 | 20.72 | / | / |
| BCMA-03 VL1VH3a | 3495 | 16.19 | 10303 | 12.13 |
| BCMA-03 VL2VH1 | 3315 | 22.29 | / | / |
| BCMA-03 VL2VH2 | 3365 | 17.63 | / | / |
| BCMA-03 VL2VH3 | 3387 | 18.87 | / | / |
| BCMA-03 VL2VH3a | 3530 | 25.15 | 9219 | 14.06 |
| BCMA-03 VL3VH1 | 3348 | 19.73 | / | / |
| BCMA-03 VL3VH2 | 3326 | 17.69 | / | / |
| BCMA-03 VL3VH3 | 3391 | 20.83 | / | / |
| BCMA-03 VL3VH3a | 3446 | 18.77 | 10103 | 12.20 |
| BCMA-mAb03 | 3632 | 19.03 | 7589 | 7.56 |
| BCMA-04 VL1VH1 | 2984 | 62.75 | / | / |
| BCMA-04 VL1VH2 | 3005 | 43.74 | / | / |
| BCMA-04 VL1VH3 | 2974 | 66.50 | / | / |
| BCMA-04 VL1VH3a | 3395 | 37.88 | 7257 | 33.19 |
| BCMA-04 VL1VH4 | 3154 | 39.08 | / | / |
| BCMA-04 VL1VH5 | 3081 | 36.15 | / | / |
| BCMA-04 VL2VH1 | 3600 | 14.42 | / | / |
| BCMA-04 VL2VH2 | 3532 | 17.28 | 3463 | 21.18 |
| BCMA-04 VL2VH3a | 3722 | 16.16 | 8849 | 10.99 |
| BCMA-04 VL2VH4 | 3493 | 23.24 | / | / |
| BCMA-04 VL2VH5 | 3347 | 23.95 | / | / |
| BCMA-mAb04 | 2982 | 47.62 | 8645 | 27.24 |
| REGN5459-hIgG1 | 4241 | 9.81 | 10139 | 5.56 |
| hIgG1 | 101 | Negative | 90 | Negative |
| BCMA-02 VL3VH8 | 2726 | 21.59 | 14102 | 16.45 |
| BCMA-02 VL3VH9 | 2764 | 42.45 | 14585 | 20.58 |
| BCMA-02 VL4VH8 | 2729 | 34.24 | 15268 | 16.45 |
| BCMA-02 VL4VH9 | 2617 | 51.01 | 15233 | 18.63 |
| BCMA-mAb02 | 2894 | 17.58 | 14258 | 14.93 |
| REGN5459-hIgG1 | 2834 | 19.42 | 16037 | 20.25 |
| hIgG1 | 84 | Negative | 93 | Negative |
| BCMA-05 VL1VH1 | 2551 | 13.20 | 14722 | 8.79 |
| BCMA-05 VL1VH2 | 2622 | 12.00 | 15103 | 7.78 |
| BCMA-05 VL1VH2a | 2637 | 9.27 | 15121 | 6.54 |
| BCMA-05 VL1VH3 | 2624 | 11.18 | 15319 | 7.61 |
| BCMA-05 VL1VH4 | 2451 | 14.80 | 14817 | 9.51 |
| BCMA-05 VL2VH1 | 2346 | 21.74 | 14541 | 11.54 |
| BCMA-05 VL2VH2 | 2428 | 18.14 | 14654 | 9.07 |
| BCMA-05 VL2VH2a | 2446 | 16.65 | 14370 | 8.72 |
| BCMA-05 VL2VH3 | 2466 | 20.12 | 14655 | 9.72 |
| BCMA-05 VL2VH4 | 2211 | 24.99 | 12985 | 11.23 |
| BCMA-05 VL3VH1 | 1790 | 17.18 | 12017 | 15.08 |
| BCMA-05 VL3VH2 | 2172 | 34.51 | 13369 | 20.41 |
| BCMA-05 VL3VH2a | 2244 | 18.36 | 14084 | 15.40 |
| BCMA-05 VL3VH3 | 2274 | 24.11 | 13612 | 14.79 |
| BCMA-05 VL3VH4 | 2002 | 22.01 | 12878 | 27.8 |
| BCMA-mAb05 | 2367 | 10.33 | 14209 | 9.829 |
| REGN5459-hIgG1 | 2654 | 9.49 | 15256 | 6.46 |
| hIgG1 | 119 | NB | 167 | NB |
| BCMA-06 VL1aVH1 | 1713 | 67.28 | 17394 | 11.99 |
| BCMA-06 VL1aVH2 | 4028 | 527.50 | 19801 | 13.44 |
| BCMA-06 VL1aVH3 | 4880 | The fit is poor | 24221 | 5.93 |
| BCMA-06 VL2aVH1 | 1305 | 47.14 | 16761 | 32.79 |
| BCMA-06 VL2aVH2 | 993 | 14.44 | 17183 | 29.65 |
| BCMA-06 VL2aVH3 | 2569 | 67.61 | 20810 | 17.8 |
| BCMA-mAb06 | 2403 | 2.83 | 21932 | 4.432 |
| BCMA-07 VL1VH2 | 4248 | The fit is poor | 20678 | 126.30 |
| BCMA-07 VL1VH3 | 1425 | 61.03 | 14503 | 55.92 |
| BCMA-07 VL2VH1 | 14390 | The fit is poor | 32484 | 134.5 |
| BCMA-07 VL2VH2 | 10989 | The fit is poor | 30747 | 207.30 |
| BCMA-07 VL2VH3 | 4165 | 376.2 | 20648 | 28.16 |
| BCMA-07 VL3VH1 | 42702 | The fit is poor | 73064 | The fit is poor |
| BCMA-07 VL3VH2 | 33494 | The fit is poor | 73193 | The fit is poor |
| BCMA-07 VL3VH3 | 24783 | The fit is poor | 52894 | The fit is poor |
| BCMA-07 VL4VH2 | 11469 | The fit is poor | 33505 | 108.4 |
| BCMA-07 VL4VH3 | 2699 | 40.07 | 19894 | 23.10 |
| BCMA-mAb07 | 1797 | 8.10 | 20968 | 9.09 |
| REGN5459-hIgG1 | 2170 | 18.21 | 18933 | 8.43 |
| hIgG1 | 98 | NB | 111 | NB |

### (C) Ligand Binding Competition ELISA Detection of Blocking Effect of Humanized Antibodies on the Binding of Ligand APRIL to Human BCMA Protein

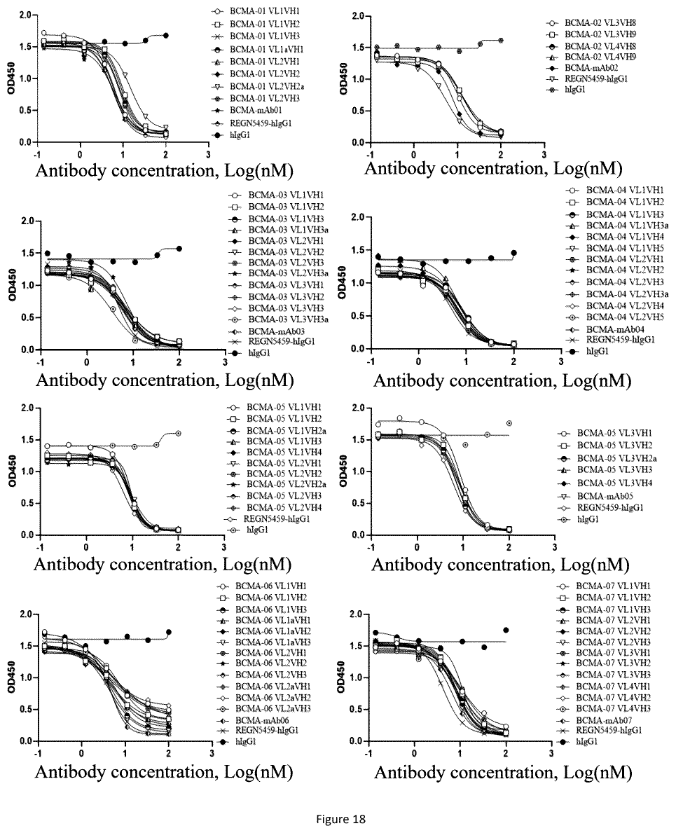

The specific method is the same as that in Example 5 (D). The results are shown in FIG. 18. All humanized antibodies have good blocking effects on the binding of the ligand APRIL to the human BCMA protein.

### Example 10 Detection of Affinity of BCMA Humanized Antibodies

The specific method was the same as that in Example 6. The binding rate (Kₐ), dissociation rate (K_{dis}) and binding affinity (KD) of the humanized antibodies to human BCMA protein are shown Table 38, wherein REGN5459-hlgG1 antibody is used as a control. As shown in Table 38, among the tested humanized antibodies, except for the KD values of humanized antibodies of BCMA-06 for human BCMA protein are below 1E-7M, the KD values of other humanized antibodies for human BCMA protein are below 1E-8M.

TABLE 38
Binding affinity of humanized antibodies to human BCMA protein

| Designation of antibody | ka (1/Ms) | kd (1/s) | KD (M) |
|---|---|---|---|
| BCMA-01 VL1VH1 | 1.74E+06 | 6.70E−04 | 3.84E−10 |
| BCMA-01 VL1VH2 | 1.92E+06 | 6.14E−04 | 3.20E−10 |
| BCMA-01 VL1aVH1 | 1.81E+06 | 6.21E−04 | 3.44E−10 |
| BCMA-01 VL2VH1 | 1.77E+06 | 6.55E−04 | 3.70E−10 |
| BCMA-01 VL2VH2 | 1.87E+06 | 6.38E−04 | 3.41E−10 |
| BCMA-01 VL2VH2a | 1.66E+06 | 7.56E−04 | 4.55E−10 |
| BCMA-01 VL2VH3 | 1.88E+06 | 6.05E−04 | 3.21E−10 |
| BCMA-mAb01 | 1.59E+06 | 8.14E−04 | 5.11E−10 |
| BCMA-03 VL1VH3a | 1.30E+06 | 2.87E−04 | 2.20E−10 |
| BCMA-03 VL2VH3a | 1.23E+06 | 2.62E−04 | 2.13E−10 |
| BCMA-03 VL3VH3a | 1.11E+06 | 2.61E−04 | 2.36E−10 |
| BCMA-mAb03 | 1.27E+06 | 1.45E−04 | 1.14E−10 |
| BCMA-04 VL1VH3a | 9.87E+05 | 1.14E−04 | 1.15E−10 |
| BCMA-04 VL2VH3a | 9.26E+05 | 1.14E−04 | 1.24E−10 |
| BCMA-mAb04 | 1.07E+06 | 1.04E−04 | 9.70E−11 |
| BCMA-02 VL3VH8 | 8.63E+05 | 3.56E−04 | 4.12E−10 |
| BCMA-02 VL3VH9 | 8.58E+05 | 3.56E−04 | 4.15E−10 |
| BCMA-02 VL4VH8 | 1.08E+06 | 3.40E−04 | 3.14E−10 |
| BCMA-02 VL4VH9 | 1.09E+06 | 3.34E−04 | 3.07E−10 |
| BCMA-mAb02 | 1.16E+06 | 2.72E−04 | 2.36E−10 |
| BCMA-05 VL1VH2a | 9.42E+05 | 2.43E−05 | 2.58E−11 |
| BCMA-05 VL2VH2a | 9.75E+05 | 2.60E−05 | 2.67E−11 |
| BCMA-05 VL3VH2a | 9.47E+05 | 2.67E−05 | 2.81E−11 |
| BCMA-mAb05 | 1.29E+06 | 2.39E−05 | 1.86E−11 |
| BCMA-06 VL1aVH1 | 2.76E+05 | 5.32E−03 | 1.93E−08 |
| BCMA-06 VL1aVH2 | 2.71E+05 | 5.01E−03 | 1.85E−08 |
| BCMA-06 VL1aVH3 | 3.82E+05 | 3.26E−03 | 8.54E−09 |
| BCMA-mAb06 | 9.52E+05 | 3.41E−04 | 3.58E−10 |
| BCMA-07 VL2VH2 | 1.19E+06 | 9.46E−04 | 7.92E−10 |
| BCMA-07 VL3VH1 | 9.13E+05 | 1.48E−03 | 1.62E−09 |
| BCMA-07 VL4VH2 | 1.47E+06 | 7.47E−04 | 5.08E−10 |
| BCMA-mAb07 | 1.42E+06 | 1.07E−03 | 7.51E−10 |
| REGN5459-hIgG1 | 7.66E+05 | 1.78E−04 | 2.33E−10 |

1. An antibody or an antigen-binding fragment thereof that specifically binds to B cell maturation antigen (BCMA), wherein the antibody or the antigen-binding fragment thereof comprises a light chain variable region (VL) and a heavy chain variable region (VH), wherein
(1) the light chain variable region comprises LCDR1, LCDR2 and LCDR3, the LCDR1 has any of a sequence of the following LCDR1, or a sequence with 1, 2, 3 or more amino acid insertions, deletions and/or substitutions compared to the sequence, the LCDR2 has any of a sequence of the following LCDR2, or a sequence with 1, 2, 3 or more amino acid insertions, deletions and/or substitutions compared to the sequence, and the LCDR3 has any of a sequence of the following LCDR3, or a sequence with 1, 2, 3 or more amino acid insertions, deletions and/or substitutions compared to the sequence:

| No. | LCDR1 | LCDR2 | LCDR3 |
|---|---|---|---|
| VL1 | SEQ ID NO: 107 | SEQ ID NO: 108 | SEQ ID NO: 109 |
| VL2 | SEQ ID NO: 110 | SEQ ID NO: 111 | SEQ ID NO: 109 |
| VL3 | SEQ ID NO: 118 | SEQ ID NO: 119 | SEQ ID NO: 120 |
| VL4 | SEQ ID NO: 121 | SEQ ID NO: 122 | SEQ ID NO: 120 |
| VL5 | SEQ ID NO: 129 | SEQ ID NO: 130 | SEQ ID NO: 131 |
| VL6 | SEQ ID NO: 132 | SEQ ID NO: 133 | SEQ ID NO: 131 |
| VL7 | SEQ ID NO: 140 | SEQ ID NO: 141 | SEQ ID NO: 142 |
| VL8 | SEQ ID NO: 143 | SEQ ID NO: 144 | SEQ ID NO: 142 |
| VL9 | SEQ ID NO: 151 | SEQ ID NO: 152 | SEQ ID NO: 153 |
| VL10 | SEQ ID NO: 154 | SEQ ID NO: 155 | SEQ ID NO: 153 |
| VL11 | SEQ ID NO: 162 | SEQ ID NO: 163 | SEQ ID NO: 164 |
| VL12 | SEQ ID NO: 165 | SEQ ID NO: 166 | SEQ ID NO: 164 |
| VL13 | SEQ ID NO: 173 | SEQ ID NO: 174 | SEQ ID NO: 175 |
| VL14 | SEQ ID NO: 176 | SEQ ID NO: 177 | SEQ ID NO: 175 |
| VL15 | SEQ ID NO: 178 | SEQ ID NO: 108 | SEQ ID NO: 109 |
| VL16 | SEQ ID NO: 162 | SEQ ID NO: 163 | SEQ ID NO: 183 |
| VL17 | SEQ ID NO: 162 | SEQ ID NO: 163 | SEQ ID NO: 184 |

and,
(2) the heavy chain variable region comprises HCDR1, HCDR2 and HCDR3, the HCDR1 has any of a sequence of the following HCDR1, or a sequence with 1, 2, 3 or more amino acid insertions, deletions and/or substitutions compared to the sequence, the HCDR2 has any of a sequence of the following HCDR2, or a sequence with 1, 2, 3 or more amino acid insertions, deletions and/or substitutions compared to the sequence, and the HCDR3 has any of a sequence of the following HCDR3, or a sequence with 1, 2, 3 or more amino acid insertions, deletions and/or substitutions compared to the sequence:

| No. | HCDR1 | HCDR2 | HCDR3 |
|---|---|---|---|
| VH1 | SEQ ID NO: 101 | SEQ ID NO: 102 | SEQ ID NO: 103 |
| VH2 | SEQ ID NO: 104 | SEQ ID NO: 105 | SEQ ID NO: 106 |
| VH3 | SEQ ID NO: 112 | SEQ ID NO: 113 | SEQ ID NO: 114 |
| VH4 | SEQ ID NO: 115 | SEQ ID NO: 116 | SEQ ID NO: 117 |
| VH5 | SEQ ID NO: 123 | SEQ ID NO: 124 | SEQ ID NO: 125 |
| VH6 | SEQ ID NO: 126 | SEQ ID NO: 127 | SEQ ID NO: 128 |
| VH7 | SEQ ID NO: 134 | SEQ ID NO: 135 | SEQ ID NO: 136 |
| VH8 | SEQ ID NO: 137 | SEQ ID NO: 138 | SEQ ID NO: 139 |
| VH9 | SEQ ID NO: 145 | SEQ ID NO: 146 | SEQ ID NO: 147 |
| VH10 | SEQ ID NO: 148 | SEQ ID NO: 149 | SEQ ID NO: 150 |
| VH11 | SEQ ID NO: 156 | SEQ ID NO: 157 | SEQ ID NO: 158 |
| VH12 | SEQ ID NO: 159 | SEQ ID NO: 160 | SEQ ID NO: 161 |
| VH13 | SEQ ID NO: 167 | SEQ ID NO: 168 | SEQ ID NO: 169 |
| VH14 | SEQ ID NO: 170 | SEQ ID NO: 171 | SEQ ID NO: 172 |
| VH15 | SEQ ID NO: 101 | SEQ ID NO: 179 | SEQ ID NO: 103 |
| VH16 | SEQ ID NO: 123 | SEQ ID NO: 180 | SEQ ID NO: 125 |
| VH17 | SEQ ID NO: 134 | SEQ ID NO: 181 | SEQ ID NO: 136 |
| VH18 | SEQ ID NO: 145 | SEQ ID NO: 182 | SEQ ID NO: 147 |

2. The antibody or the antigen-binding fragment thereof of claim 1, wherein:
(1) the light chain variable region comprises a sequence as shown in any one of SEQ ID NO: 8, 10, 12, 14, 16, 18, 20, 21-23, 32-33, 41-43, 52-53, 65-67, 78-81 and 90-93, or a sequence having 80%, 85%, 90%, 95%, 96%, 97%, 98%, 99% or higher identity to the sequence; and,
(2) the heavy chain variable region comprises a sequence as shown in any one of SEQ ID NO: 7, 9, 11, 13, 15, 17, 19, 24-27, 34-35, 44-47, 54-59, 68-72, 82-84 and 94-96, or a sequence having 80%, 85%, 90%, 95%, 96%, 97%, 98%, 99% or higher identity to the sequence.

3. The antibody or the antigen-binding fragment thereof of claim 1, wherein, the antibody or the antigen-binding fragment thereof is chimeric, humanized or fully human.

4. The antibody or the antigen-binding fragment thereof of claim 1, wherein, the antibody or the antigen-binding fragment thereof binds to human or monkey BCMA.

5. The antibody or the antigen-binding fragment thereof of claim 1, wherein, the antibody or the antigen-binding fragment thereof binds to human BCMA with a dissociation constant of no greater than 10-7 M or 10-8 M; or the antibody or the antigen-binding fragment thereof blocks the binding of APRIL to human BCMA.

6. (canceled)

7. The antibody or the antigen-binding fragment thereof of claim 1, wherein, the antibody or the antigen-binding fragment thereof comprises a constant region sequence of human or murine antibody lgG1, lgG2, lgG3, lgG4, IgA, IgM, IgE or IgD.

8. The antibody or the antigen-binding fragment thereof of claim 1, wherein, the antigen-binding fragment is selected from one or more of F(ab′)2, Fab′, Fab, Fv, scFv, nanobody or affibody.

9. A multispecific antigen-binding molecule, wherein the multispecific antigen-binding molecule comprises the antibody or the antigen-binding fragment thereof of claim 1, and an antigen-binding molecule binding to an additional antigen other than BCMA or binding to a different BCMA epitope from that of the antibody or the antigen-binding fragment thereof of claim 1; optionally, the additional antigen other than BCMA is selected from: CD3, CD16, CD137, CD258, PD-1, PD-L1, 4-1BB, CD40, CD64, EGFR, VEGF, HER2, HER1, HER3, IGF-1R, phosphatidylserine (PS), C-Met, HSA, GPRC5D, MSLN, blood-brain barrier receptor, GPC3, PSMA, CD33, GD2, ROR1, ROR2, FRα or Gucy2C.

10. A chimeric antigen receptor (CAR), wherein the chimeric antigen receptor comprises at least a signal peptide, an extracellular antigen-binding domain, a hinge region, a transmembrane domain and an intracellular signaling domain, and the extracellular antigen-binding domain comprises the BCMA antibody or the antigen-binding fragment thereof of claim 1; or the multispecific antigen-binding molecule which comprises the antibody or the antigen-binding fragment thereof of claim 1, and an antigen-binding molecule binding to an additional antigen other than BCMA or binding to a different BCMA epitope from that of the antibody or the antigen-binding fragment thereof claim 1.

11. An immune effector cell, wherein the immune effector cell expresses the chimeric antigen receptor of claim 10, or comprises a nucleic acid fragment encoding the chimeric antigen receptor of claim 10.

12. An isolated nucleic acid fragment, wherein the nucleic acid fragment encodes the antibody or the antigen-binding fragment thereof of claim 1; the multispecific antigen-binding molecule which comprises the antibody or the antigen-binding fragment thereof of claim 1, and an antigen-binding molecule binding to an additional antigen other than BCMA or binding to a different BCMA epitope from that of the antibody or the antigen-binding fragment thereof of claim 1; or the chimeric antigen receptor which comprises at least a signal peptide, an extracellular antigen-binding domain, a hinge region, a transmembrane domain and an intracellular signaling domain, wherein the extracellular antigen-binding domain comprises the BCMA antibody or the antigen-binding fragment thereof, or the multispecific antigen-binding molecule.

13. A vector, wherein the vector comprises the nucleic acid fragment of claim 12.

14. A host cell, wherein the host cell comprises the vector of claim 13.

15. A method of preparing the antibody or the antigen-binding fragment thereof of claim 1 or the multispecific antigen-binding molecule which comprises the antibody or the antigen-binding fragment thereof of claim 1 and an antigen-binding molecule binding to an additional antigen other than BCMA or binding to a different BCMA epitope from that of the antibody or the antigen-binding fragment thereof of claim 1, wherein, the method comprises culturing the cell comprising a vector which comprises a nucleic acid fragment encoding the antibody or the antigen binding fragment thereof, or the multi-specific antigen binding molecule, and isolating the antibody, the antigen-binding fragment or the multispecific antigen-binding molecule expressed by the cell.

16. A method of preparing the immune effector cell of claim 11, wherein the method comprises introducing the nucleic acid fragment encoding the CAR of claim 11 into the immune effector cell, optionally, the method further comprises enabling the immune effector cell to express the aforementioned CAR.

17. A pharmaceutical composition, wherein the pharmaceutical composition comprises the antibody or the antigen-binding fragment thereof of claim 1; the multispecific antigen-binding molecule which comprises the antibody or the antigen-binding fragment thereof of claim 1 and an antigen-binding molecule binding to an additional antigen other than BCMA or binding to a different BCMA epitope from that of the antibody or the antigen-binding fragment thereof of claim 1; the immune effector cell expressing a chimeric antigen receptor which comprises at least a signal peptide, an extracellular antigen-binding domain, a hinge region, a transmembrane domain and an intracellular signaling domain, wherein the extracellular antigen-binding domain comprises the BCMA antibody or the antigen-binding fragment thereof or the multispecific antigen-binding molecule; the nucleic acid fragment encoding the antibody or the antigen-binding fragment thereof, the multispecific antigen-binding molecule or the chimeric antigen receptor; or the vector comprising the nucleic acid fragment; optionally, the pharmaceutical composition further comprises a pharmaceutically acceptable carrier, a diluent or an auxiliary agent; and optionally, the pharmaceutical composition further comprises an additional anti-tumor agent.

18. A method of treating a tumor or a cancer, wherein the method comprises administering to a subject an effective amount of the antibody or the antigen-binding fragment thereof of claim 1; the multispecific antigen-binding molecule which comprises the antibody or the antigen-binding fragment thereof of claim 1 and an antigen-binding molecule binding to an additional antigen other than BCMA or binding to a different BCMA epitope from that of the antibody or the antigen-binding fragment thereof of claim 1; the immune effector cell expressing a chimeric antigen receptor which comprises at least a signal peptide, an extracellular antigen-binding domain, a hinge region, a transmembrane domain and an intracellular signaling domain, wherein the extracellular antigen-binding domain comprises the BCMA antibody or the antigen-binding fragment thereof or the multispecific antigen-binding molecule; the nucleic acid fragment encoding the antibody or the antigen-binding fragment thereof, the multispecific antigen-binding molecule or the chimeric antigen receptor; or the vector comprising the nucleic acid fragment; the tumor or the cancer is a BCMA-expressing tumor or cancer.

19-20. (canceled)

21. A kit, wherein the kit comprises the antibody or the antigen-binding fragment thereof of claim 1; the multispecific antigen-binding molecule which comprises the antibody or the antigen-binding fragment thereof of claim 1 and an antigen-binding molecule binding to an additional antigen other than BCMA or binding to a different BCMA epitope from that of the antibody or the antigen-binding fragment thereof of claim 1; the immune effector cell expressing a chimeric antigen receptor which comprises at least a signal peptide, an extracellular antigen-binding domain, a hinge region, a transmembrane domain and an intracellular signaling domain, wherein the extracellular antigen-binding domain comprises the BCMA antibody or the antigen-binding fragment thereof or the multispecific antigen-binding molecule; the nucleic acid fragment encoding the antibody or the antigen-binding fragment thereof, the multispecific antigen-binding molecule or the chimeric antigen receptor; or the vector comprising the nucleic acid fragment.

22. A method of detecting BCMA expression in a biological sample, wherein the method comprises contacting the biological sample with the antibody or the antigen-binding fragment thereof of claim 1 under conditions that allow the formation of a complex from the antibody or the antigen-binding fragment thereof and BCMA.

23. Use of the antibody or the antigen-binding fragment thereof of claim 1 in the preparation of a reagent for detecting BCMA.

24. The antibody or the antigen-binding fragment thereof of claim 2, wherein the antibody or the antigen-binding fragment thereof has a light chain variable region and a heavy chain variable region as follows:
(1) the light chain variable region and the heavy chain variable region comprise sequences as shown in SEQ ID NO: 8 and SEQ ID NO: 7, respectively;
(2) the light chain variable region and the heavy chain variable region comprise sequences as shown in SEQ ID NO: 10 and SEQ ID NO: 9, respectively;
(3) the light chain variable region and the heavy chain variable region comprise sequences as shown in SEQ ID NO: 12 and SEQ ID NO: 11, respectively;
(4) the light chain variable region and the heavy chain variable region comprise sequences as shown in SEQ ID NO: 14 and SEQ ID NO: 13, respectively;
(5) the light chain variable region and the heavy chain variable region comprise sequences as shown in SEQ ID NO: 16 and SEQ ID NO: 15, respectively;
(6) the light chain variable region and the heavy chain variable region comprise sequences as shown in SEQ ID NO: 18 and SEQ ID NO: 17, respectively;
(7) the light chain variable region and the heavy chain variable region comprise sequences as shown in SEQ ID NO: 20 and SEQ ID NO: 19, respectively;
(8) the light chain variable region comprises a sequence as shown in any one of SEQ ID NOs: 21-23, and the heavy chain variable region comprises a sequence as shown in any one of SEQ ID NOs: 24-27;
(9) the light chain variable region comprises a sequence as shown in any one of SEQ ID NOs: 32-33, and the heavy chain variable region comprises a sequence as shown in any one of SEQ ID NOs: 34-35;
(10) the light chain variable region comprises a sequence as shown in any one of SEQ ID NOs: 41-43, and the heavy chain variable region comprises a sequence as shown in any one of SEQ ID NOs: 44-47;
(11) the light chain variable region comprises a sequence as shown in any one of SEQ ID NOs: 52-53, and the heavy chain variable region comprises a sequence as shown in any one of SEQ ID NOs: 54-59;
(12) the light chain variable region comprises a sequence as shown in any one of SEQ ID NOs: 65-67, and the heavy chain variable region comprises a sequence as shown in any one of SEQ ID NOs: 68-72;
(13) the light chain variable region comprises a sequence as shown in any one of SEQ ID NOs: 78-81, and the heavy chain variable region comprises a sequence as shown in any one of SEQ ID NOs: 82-84;
(14) the light chain variable region comprises a sequence as shown in any one of SEQ ID NOs: 90-93, and the heavy chain variable region comprises a sequence as shown in any one of SEQ ID NOs: 94-96;
or
(15) the light chain variable region comprises a sequence having 80%, 85%, 90%, 95%, 96%, 97%, 98%, 99% or higher identity to the light chain variable region as shown in any one of the above (1) to (14), and the heavy chain variable region comprises a sequence having 80%, 85%, 90%, 95%, 96%, 97%, 98%, 99% or higher identity to the heavy chain variable region as shown in any one of the above (1) to (14).


SEQUENCE LISTING (184 sequences)

SEQ ID NO: 1 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 2 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 3 - protein, 119 aa, synthetic construct
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 4 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 107 aa, synthetic construct [sequence omitted]

SEQ ID NO: 6 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 126 aa, synthetic construct
EVKLVESGGGLVQPGGSLKLSCAASGFTFSDFYMYWVRQTPEKRLEWVAYITNGGENTYFPDTVKGRFTISRDNAKNTLYLQMSRLKSEDTAMYYCARQNPFYYYDSNYDYAMDFWGQGTSVTVSS

SEQ ID NO: 8 - protein, 112 aa, synthetic construct
DVVMTQTPLTLSVTIGQPASISCKSSQSLLYSNGKTYLSWLFQRPGQSPKRLIYLVSELDSGVPDRFTGSGSGTDFTLKISRVEAEDLGIYYCVQGTHFPHTFGGGTKLEIK

SEQ ID NO: 9 - protein, 121 aa, synthetic construct
EVQLVESGGDLVQPGESLKLSCESNAYEFPSYDMSWVRKTPEKRLELVAAINSDGGITYYPDTMERRFIISRDNTKKTLYLQMSSLRSEDTALYYCARHGYGGGAYVLDYWGQGTSVTVSS

SEQ ID NO: 10 - protein, 107 aa, synthetic construct
DIVMTQSQKFMSTTVGDRVRITCKASQNVGTAVAWYQQKPGQSPKVLIYSASSRYTGVPDRFTGSGSGTDFTLTINNMQSEDLADYFCQQYSNYHLTFGTGTKLELK

SEQ ID NO: 11 - protein, 121 aa, synthetic construct
QIPLVQSGPELKKPGETVKISCKASGYTFTNYAMTWVKQAPGKGLKWMGWIDTYSGVPTYADDFKGRFAFSLETSATTAYLQINNLKNEDTATYFCARVGVYGGYDTLDSWGQGTSVTVSS

SEQ ID NO: 12 - protein, 107 aa, synthetic construct
DIQMTQTTSSLSASLGDRVTISCSASQGISNHLNWYQQKPDGTVKLLIYYTSSLHSGVPSRFSGSGSGTDYSLTISNLEPEDFATYYCQQYNNLPWTFGGGTKLEIK

SEQ ID NO: 13 - protein, 123 aa, synthetic construct
QVQLQQSGAELARPGASVDLSCKASGYSFTSYGISWVKLRTGQGLEWIGEIYPRDDITYYNEKFKGKTTLTADKSSSTAYMELRSLTSEDSAVYFCARHDYYYVNSVSAMDYWGQGTSVTVSS

SEQ ID NO: 14 - protein, 113 aa, synthetic construct
DIVMSQSPSSLAVSVGEKVTMSCKSSQSLLYSSNQKNYLAWYQQKPGQSPKLLIYWASTRYSGVPDRFTGSGSGTDFTLTVSSVRAEDLALYYCQQYYIYPWTFGGGTKLEIK

SEQ ID NO: 15 - protein, 121 aa, synthetic construct
QGQMQQSDAELVKPGASVQISCKASGYMFTDHTFHWMRQRPEQGLEWIGYIYPRDDNTKYNEKFKGKATLTADKSSSTAYIHLNSLTSEDSAVYFCARAGYYGSSHAMDYWGQGTSVTVSS

SEQ ID NO: 16 - protein, 107 aa, synthetic construct
DIVMTQSHKFMSTSVGDRVSITCKASQDVSTAVAWYQQKPGQSPRLLIYSASSRYTGVPDRFTGSGSGTDFTFTISSVQTEDLAVYYCQQLYSTPWTFGGGTKLEIK

SEQ ID NO: 17 - protein, 120 aa, synthetic construct
EVKLLQSGGGLVQPGGSLKLSCAASGIDFSRYWMSWVRRAPGKGLEWIGEINPDSSTINYAPSLKDEFIISRDNAKNTLYLQMSKVRSEDTALYYCAIFYYANYYAMDYWGQGTSVTVSS

SEQ ID NO: 18 - protein, 107 aa, synthetic construct
DIVMTQSQKFMSTSVGDRVSVTCKASQNVDTNVAWYQQKPGQSPKALIYSASYRFSGVPDRFTGSGSGTDFTLTISNVQSEDLAEYFCQQYNGYPFTFASGTKLEIK

SEQ ID NO: 19 - protein, 118 aa, synthetic construct
QVQLQQSGAELAKPGASVKLSCKASGYTFTDYWTHWVKQRPGQGLEWIGYINHGSGYTKYNQKFKDKATLTADKSSSTAYMQVSSLTYEDSAVYYCARLTGSYYFDYWGQGTTLTVSS

SEQ ID NO: 20 - protein, 106 aa, synthetic construct
QIVLTQSPAIMSASPGEKVTMTCSASSSVSYMHWYQQKSGTSPKRWIYDTSKLASGVPPRFSGSGSGTSYSLTISSMEAEDAATYYCQQWSSNPLTFGAGTKLELK

SEQ ID NO: 21 - protein, 112 aa, synthetic construct
DVVMTQSPLSLPVTLGQPASISCKSSQSLLYSNGKTYLSWLFQRPGQSPRRLIYLVSELDSGVPDRFSGSGSGTDFTLKISRVEAEDVGVYYCVQGTHFPHTFGGGTKVEIK

SEQ ID NO: 22 - protein, 112 aa, synthetic construct
DVVMTQSPLSLPVTLGQPASISCKSSQSLLYSNAKTYLSWLFQRPGQSPRRLIYLVSELDSGVPDRFSGSGSGTDFTLKISRVEAEDVGVYYCVQGTHFPHTFGGGTKVEIK

SEQ ID NO: 23 - protein, 112 aa, synthetic construct
DVVMTQSPLSLPVTLGQPASISCKSSQSLLYSNGKTYLSWLFQRPGQSPKRLIYLVSELDSGVPDRFSGSGSGTDFTLKISRVEAEDVGVYYCVQGTHFPHTFGGGTKVEIK

SEQ ID NO: 24 - protein, 126 aa, synthetic construct
EVQLVESGGGLVQPGGSLRLSCAASGFTFSDFYMYWVRQAPGKGLEWVAYITNGGENTYFPDTVKGRFTISRDNAKNSLYLQMNSLRAEDTAVYYCARQNPFYYYDSNYDYAMDFWGQGTTVTVSS

SEQ ID NO: 25 - protein, 126 aa, synthetic construct
EVQLVESGGGLVQPGGSLRLSCAASGFTFSDFYMYWVRQAPEKRLEWVAYITNGGENTYFPDTVKGRFTISRDNAKNSLYLQMNSLRAEDTAVYYCARQNPFYYYDSNYDYAMDFWGQGTTVTVSS

SEQ ID NO: 26 - protein, 126 aa, synthetic construct
EVQLVESGGGLVQPGGSLRLSCAASGFTFSDFYMYWVRQAPEKRLEWVAYITNAGENTYFPDTVKGRFTISRDNAKNSLYLQMNSLRAEDTAVYYCARQNPFYYYDSNYDYAMDFWGQGTTVTVSS

SEQ ID NO: 27 - protein, 126 aa, synthetic construct
EVKLVESGGGLVQPGGSLRLSCAASGFTFSDFYMYWVRQAPEKRLEWVAYITNGGENTYFPDTVKGRFTISRDNAKNSLYLQMNSLRAEDTAVYYCARQNPFYYYDSNYDYAMDFWGQGTTVTVSS

SEQ ID NO: 28 - protein, 100 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 29 - protein, 10 aa, synthetic construct
FGGGTKVEIK

SEQ ID NO: 30 - protein, 98 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 31 - protein, 11 aa, synthetic construct
WGQGTTVTVSS

SEQ ID NO: 32 - protein, 107 aa, synthetic construct
DIQMTQSPSSVSASVGDRVTITCKASQNVGTAVAWYQQKPGKSPKVLIYSASSRYTGVPSRFSGSGSGTDFTLTISSLQPEDFADYYCQQYSNYHLTFGQGTKLEIK

SEQ ID NO: 33 - protein, 107 aa, synthetic construct
DIVMTQSPDSLAVSLGERATINCKASQNVGTAVAWYQQKPGQSPKVLIYSASSRYTGVPDRFSGSGSGTDFTLTISSLQAEDVAVYYCQQYSNYHLTFGQGTKLEIK

SEQ ID NO: 34 - protein, 121 aa, synthetic construct
EVQLVESGGGVVQPGRSLRLSCASNAYEFPSYDMSWVRQAPGKGLELVAAINSDGGITYYPDTMERRFTISRDNTKKTLYLQMSSLRAEDTAVYYCARHGYGGGAYVLDYWGQGTTVTVSS

SEQ ID NO: 35 - protein, 121 aa, synthetic construct
EVQLVESGGGVVQPGRSLRLSCESNAYEFPSYDMSWVRQAPGKGLELVAAINSDGGITYYPDTMERRFTISRDNTKKTLYLQMSSLRAEDTAVYYCARHGYGGGAYVLDYWGQGTTVTVSS

SEQ ID NO: 36 - protein, 95 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 37 - protein, 101 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 38 - protein, 10 aa, synthetic construct
FGQGTKLEIK

SEQ ID NO: 39 - protein, 98 aa, synthetic construct
QVQLVESGGGVVQPGRSLRLSCAASGFTFSSYAMHWVRQAPGKGLEWVAVISYDGSNKYYADSVKGRFTISRDNSKNTLYLQMSSLRAEDTAVYYCAR

SEQ ID NO: 40 - protein, 11 aa, synthetic construct
WGQGTTVTVSS

SEQ ID NO: 41 - protein, 107 aa, synthetic construct
DIQMTQSPSSLSASVGDRVTITCSASQGISNHLNWYQQKPGKAVKLLIYYTSSLHSGVPSRFSGSGSGTDFTLTISSLQPEDFATYYCQQYNNLPWTFGGGTKVEIK

SEQ ID NO: 42 - protein, 107 aa, synthetic construct
DIQMTQSPSSLSASVGDRVTITCSASQGISNHLNWYQQKPDKTVKLLIYYTSSLHSGVPSRFSGSGSGTDFTLTISSLQPEDFATYYCQQYNNLPWTFGGGTKVEIK

SEQ ID NO: 43 - protein, 107 aa, synthetic construct
DIQMTQSPSSLSASVGDRVTITCSASQGISNHLNWYQQKPDGTVKLLIYYTSSLHSGVPSRFSGSGSGTDFTLTISSLQPEDFATYYCQQYNNLPWTFGGGTKVEIK

SEQ ID NO: 44 - protein, 121 aa, synthetic construct
EVQLVQSGSELKKPGASVKVSCKASGYTFTNYAMTWVRQAPGQGLEWMGWIDTYSGVPTYADDFKGRFVFSLDTSVSTAYLQISSLKAEDTAVYYCARVGVYGGYDTLDSWGQGTTVTVSS

SEQ ID NO: 45 - protein, 121 aa, synthetic construct
EVQLVQSGSELKKPGASVKVSCKASGYTFTNYAMTWVRQAPGQGLEWMGWIDTYSGVPTYADDFKGRFVFSLETSASTAYLQISSLKAEDTAVYYCARVGVYGGYDTLDSWGQGTTVTVSS

SEQ ID NO: 46 - protein, 121 aa, synthetic construct
EVPLVQSGSELKKPGASVKVSCKASGYTFTNYAMTWVRQAPGQGLEWMGWIDTYSGVPTYADDFKGRFVFSLETSASTAYLQISSLKAEDTAVYYCARVGVYGGYDTLDSWGQGTTVTVSS

SEQ ID NO: 47 - protein, 121 aa, synthetic construct
EVPLVQSGSELKKPGASVKVSCKASGYTFTNYAMTWVRQAPGQGLEWMGWIDTYSGVPTYADSFKGRFVFSLETSASTAYLQISSLKAEDTAVYYCARVGVYGGYDTLDSWGQGTTVTVSS

SEQ ID NO: 48 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 49 - protein, 10 aa, synthetic construct
FGGGTKVEIK

SEQ ID NO: 50 - protein, 98 aa, synthetic construct
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCAR

SEQ ID NO: 51 - protein, 11 aa, synthetic construct
WGQGTTVTVSS

SEQ ID NO: 52 - protein, 113 aa, synthetic construct
DIVMTQSPDSLAVSLGERATINCKSSQSLLYSSNQKNYLAWYQQKPGQSPKLLIYWASTRYSGVPDRFSGSGSGTDFTLTISSLQAEDVAVYYCQQYYIYPWTFGGGTKVEIK

SEQ ID NO: 53 - protein, 113 aa, synthetic construct
DIQMTQSPSSLSASVGDRVTITCKSSQSLLYSSNQKNYLAWYQQKPGKSPKLLIYWASTRYSGVPSRFSGSGSGTDFTLTISSLQPEDFATYYCQQYYIYPWTFGGGTKVEIK

SEQ ID NO: 54 - protein, 123 aa, synthetic construct
EVQLVQSGAEVKKPGSSVKVSCKASGYTFSSYGISWVRQAPGQGLEWMGEIYPRDDITYYNEKFKGRVTITADKSTSTAYMELSSLRSEDTAVYYCARHDYYYVNSVSAMDYWGQGTTVTVSS

SEQ ID NO: 55 - protein, 123 aa, synthetic construct
EVQLVQSGAEVKKPGSSVKVSCKASGYSFTSYGISWVRQAPGQGLEWMGEIYPRDDITYYNEKFKGRVTITADKSTSTAYMELSSLRSEDTAVYYCARHDYYYVNSVSAMDYWGQGTTVTVSS

SEQ ID NO: 56 - protein, 123 aa, synthetic construct
EVQLVQSGAEVKKPGSSVKVSCKASGYSFTSYGISWVRQAPGQGLEWMGEIYPRDDITYYNEKFKGRVTLTADKSTSTAYMELSSLRSEDTAVYYCARHDYYYVNSVSAMDYWGQGTTVTVSS

SEQ ID NO: 57 - protein, 123 aa, synthetic construct
EVQLVQSGAEVKKPGSSVKVSCKASGYSFTSYGISWVRQAPGQGLEWMGEIYPRDQITYYNEKFKGRVTLTADKSTSTAYMELSSLRSEDTAVYYCARHDYYYVNSVSAMDYWGQGTTVTVSS

SEQ ID NO: 58 - protein, 123 aa, synthetic construct
EVQLQQSGAEVKKPGSSVKVSCKASGYSFTSYGISWVRQAPGQGLEWMGEIYPRDDITYYNEKFKGRVTITADKSTSTAYMELSSLRSEDTAVYYCARHDYYYVNSVSAMDYWGQGTTVTVSS

SEQ ID NO: 59 - protein, 123 aa, synthetic construct
EVQLVQSGAEVKKPGSSVKVSCKASGYSFTSYGISWVRLAPGQGLEWMGEIYPRDDITYYNEKFKGRVTITADKSTSTAYMELSSLRSEDTAVYYCARHDYYYVNSVSAMDYWGQGTTVTVSS

SEQ ID NO: 60 - protein, 101 aa, synthetic construct; identical to SEQ ID NO: 37

SEQ ID NO: 61 - protein, 95 aa, synthetic construct; identical to SEQ ID NO: 48

SEQ ID NO: 62 - protein, 10 aa, synthetic construct
FGGGTKVEIK

SEQ ID NO: 63 - protein, 98 aa, synthetic construct
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAR

SEQ ID NO: 64 - protein, 11 aa, synthetic construct
WGQGTTVTVSS

SEQ ID NO: 65 - protein, 107 aa, synthetic construct
DIQMTQSPSSLSASVGDRVTITCKASQDVSTAVAWYQQKPGKSPRLLIYSASSRYTGVPSRFSGSGSGTDFTFTISSLQPEDIATYYCQQLYSTPWTFGGGTKVEIK

SEQ ID NO: 66 - protein, 107 aa, synthetic construct
DIQMTQSPSSLSASVGDRVTITCKASQDVSTAVAWYQQKPGKSPRLLIYSASSRYTGVPDRFSGSGSGTDFTFTISSLQPEDIATYYCQQLYSTPWTFGGGTKVEIK

SEQ ID NO: 67 - protein, 107 aa, synthetic construct
DIVMTQSPDSLAVSLGERATINCKASQDVSTAVAWYQQKPGQSPRLLIYSASSRYTGVPDRFSGSGSGTDFTLTISSLQAEDVAVYYCQQLYSTPWTFGGGTKVEIK

SEQ ID NO: 68 - protein, 121 aa, synthetic construct
EVQLVQSGAEVKKPGSSVKVSCKASGYMFTDHTFHWVRQAPGQGLEWMGYIYPRDDNTKYNEKFKGRVTITADKSTSTAYMELSSLRSEDTAVYYCARAGYYGSSHAMDYWGQGTTVTVSS

SEQ ID NO: 69 - protein, 121 aa, synthetic construct
EGQMVQSGAEVKKPGSSVKVSCKASGYMFTDHTFHWVRQAPGQGLEWMGYIYPRDDNTKYNEKFKGRVTITADKSTSTAYMELSSLRSEDTAVYYCARAGYYGSSHAMDYWGQGTTVTVSS

SEQ ID NO: 70 - protein, 121 aa, synthetic construct
EGQMVQSGAEVKKPGSSVKVSCKASGYMFTDHTFHWVRQAPGQGLEWMGYIYPRDQNTKYNEKFKGRVTITADKSTSTAYMELSSLRSEDTAVYYCARAGYYGSSHAMDYWGQGTTVTVSS

SEQ ID NO: 71 - protein, 121 aa, synthetic construct
EVQLVQSGAEVKKPGSSVKVSCKASGYMFTDHTFHWMRQAPGQGLEWMGYIYPRDDNTKYNEKFKGRVTITADKSTSTAYMELSSLRSEDTAVYYCARAGYYGSSHAMDYWGQGTTVTVSS

SEQ ID NO: 72 - protein, 121 aa, synthetic construct
EVQLVQSGAEVKKPGSSVKVSCKASGYMFTDHTFHWVRQAPGQGLEWMGYIYPRDDNTKYNEKFKGRVTLTADKSTSTAYMELSSLRSEDTAVYYCARAGYYGSSHAMDYWGQGTTVTVSS

SEQ ID NO: 73 - protein, 95 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 74 - protein, 101 aa, synthetic construct; identical to SEQ ID NO: 37

SEQ ID NO: 75 - protein, 10 aa, synthetic construct
FGGGTKVEIK

SEQ ID NO: 76 - protein, 98 aa, synthetic construct
QVQLVQSGAEVKKPGSSVKVSCKASGGTFSSYTISWVRQAPGQGLEWMGRIIPILGIANYAQKFQGRVTITADKSTSTAYMELSSLRSEDTAVYYCAR

SEQ ID NO: 77 - protein, 11 aa, synthetic construct
WGQGTTVTVSS

SEQ ID NO: 78 - protein, 107 aa, synthetic construct
DIQMTQSPSSVSASVGDRVTITCKASQNVDTNVAWYQQKPGKSPKALIYSASYRFSGVPSRFSGSGSGTDFTLTISSLQPEDFATYYCQQYNGYPFTFGQGTKLEIK

SEQ ID NO: 79 - protein, 107 aa, synthetic construct
DIQMTQSPSSVSASVGDRVTITCKASQNVDTNVAWYQQKPGKSPKALIYSASYRFSGVPSRFSGSGSGTDFTLTISSLQPEDFATYYCQQYQGYPFTFGQGTKLEIK

SEQ ID NO: 80 - protein, 107 aa, synthetic construct
DIVMTQSPDSLAVSLGERATINCKASQNVDTNVAWYQQKPGQSPKALIYSASYRFSGVPDRFSGSGSGTDFTLTISSLQAEDVAVYYCQQYNGYPFTFGQGTKLEIK

SEQ ID NO: 81 - protein, 107 aa, synthetic construct
DIVMTQSPDSLAVSLGERATINCKASQNVDTNVAWYQQKPGQSPKALIYSASYRFSGVPDRFSGSGSGTDFTLTISSLQAEDVAVYYCQQYNAYPFTFGQGTKLEIK

SEQ ID NO: 82 - protein, 120 aa, synthetic construct
EVQLVESGGGLVQPGGSLRLSCAASGIDFSRYWMSWVRQAPGKGLEWVAEINPDSSTINYAPSLKDRFTISRDNAKNSLYLQMNSLRAEDTAVYYCAIFYYANYYAMDYWGQGTTVTVSS

SEQ ID NO: 83 - protein, 120 aa, synthetic construct
EVKLVESGGGLVQPGGSLRLSCAASGIDFSRYWMSWVRQAPGKGLEWVAEINPDSSTINYAPSLKDRFTISRDNAKNSLYLQMNSLRAEDTAVYYCAIFYYANYYAMDYWGQGTTVTVSS

SEQ ID NO: 84 - protein, 120 aa, synthetic construct
EVKLLQSGGGLVQPGGSLRLSCAASGIDFSRYWMSWVRQAPGKGLEWVAEINPDSSTINYAPSLKDRFTISRDNAKNSLYLQMNSLRAEDTAVYYCAIFYYANYYAMDYWGQGTTVTVSS

SEQ ID NO: 85 - protein, 95 aa, synthetic construct; identical to SEQ ID NO: 36

SEQ ID NO: 86 - protein, 101 aa, synthetic construct; identical to SEQ ID NO: 37

SEQ ID NO: 87 - protein, 10 aa, synthetic construct
FGQGTKLEIK

SEQ ID NO: 88 - protein, 98 aa, synthetic construct; identical to SEQ ID NO: 30

SEQ ID NO: 89 - protein, 11 aa, synthetic construct
WGQGTTVTVSS

SEQ ID NO: 90 - protein, 106 aa, synthetic construct
DIQMTQSPSSLSASVGDRVTITCSASSSVSYMHWYQQKPGKSPKRLIYDTSKLASGVPSRFSGSGSGTEFTLTISSLQPEDFATYYCQQWSSNPLTFGQGTKLEIK

SEQ ID NO: 91 - protein, 106 aa, synthetic construct
DIQMTQSPSSLSASVGDRVTITCSASSSVSYMHWYQQKPGKSPKRWIYDTSKLASGVPSRFSGSGSGTEFTLTISSLQPEDFATYYCQQWSSNPLTFGQGTKLEIK

SEQ ID NO: 92 - protein, 106 aa, synthetic construct
QIQMTQSPSSLSASVGDRVTITCSASSSVSYMHWYQQKPGKSPKRWIYDTSKLASGVPSRFSGSGSGTEFTLTISSLQPEDFATYYCQQWSSNPLTFGQGTKLEIK

SEQ ID NO: 93 - protein, 106 aa, synthetic construct
QIQMTQSPSSLSASVGDRVTITCSASSSVSYMHWYQQKPGTSPKRWIYDTSKLASGVPSRFSGSGSGTEFTLTISSLQPEDFATYYCQQWSSNPLTFGQGTKLEIK

SEQ ID NO: 94 - protein, 118 aa, synthetic construct
EVQLVQSGAEVKKPGSSVKVSCKASGYTFTDYWTHWVRQAPGQGLEWMGYINHGSGYTKYNQKFKDRVTITADKSTSTAYMELSSLRSEDTAVYYCARLTGSYYFDYWGQGTTVTVSS

SEQ ID NO: 95 - protein, 118 aa, synthetic construct
EVQLVQSGAEVKKPGSSVKVSCKASGYTFTDYWTHWVRQAPGQGLEWMGYINHGSGYTKYNQKFKDRVTLTADKSTSTAYMELSSLRSEDTAVYYCARLTGSYYFDYWGQGTTVTVSS

SEQ ID NO: 96 - protein, 118 aa, synthetic construct
EVQLVQSGAEVKKPGSSVKVSCKASGYTFTDYWTHWVRQAPGQGLEWIGYINHGSGYTKYNQKFKDRATLTADKSTSTAYMELSSLRSEDTAVYYCARLTGSYYFDYWGQGTTVTVSS

SEQ ID NO: 97 - protein, 95 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 98 - protein, 10 aa, synthetic construct
FGQGTKLEIK

SEQ ID NO: 99 - protein, 98 aa, synthetic construct; identical to SEQ ID NO: 76

SEQ ID NO: 100 - protein, 11 aa, synthetic construct
WGQGTTVTVSS

SEQ ID NO: 101 - protein, 5 aa, synthetic construct
DFYMY

SEQ ID NO: 102 - protein, 17 aa, synthetic construct
YITNGGENTYFPDTVKG

SEQ ID NO: 103 - protein, 17 aa, synthetic construct
QNPFYYYDSNYDYAMDF

SEQ ID NO: 104 - protein, 8 aa, synthetic construct
GFTFSDFY

SEQ ID NO: 105 - protein, 8 aa, synthetic construct
ITNGGENT

SEQ ID NO: 106 - protein, 19 aa, synthetic construct
ARQNPFYYYDSNYDYAMDF

SEQ ID NO: 107 - protein, 16 aa, synthetic construct
KSSQSLLYSNGKTYLS

SEQ ID NO: 108 - protein, 7 aa, synthetic construct
LVSELDS

SEQ ID NO: 109 - protein, 9 aa, synthetic construct
VQGTHFPHT

SEQ ID NO: 110 - protein, 11 aa, synthetic construct
QSLLYSNGKTY

SEQ ID NO: 111 - skipped

SEQ ID NO: 112 - protein, 5 aa, synthetic construct
SYDMS

SEQ ID NO: 113 - protein, 17 aa, synthetic construct
AINSDGGITYYPDTMER

SEQ ID NO: 114 - protein, 12 aa, synthetic construct
HGYGGGAYVLDY

SEQ ID NO: 115 - protein, 8 aa, synthetic construct
AYEFPSYD

SEQ ID NO: 116 - protein, 8 aa, synthetic construct
INSDGGIT

SEQ ID NO: 117 - protein, 14 aa, synthetic construct
ARHGYGGGAYVLDY

SEQ ID NO: 118 - protein, 11 aa, synthetic construct
KASQNVGTAVA

SEQ ID NO: 119 - protein, 7 aa, synthetic construct
SASSRYT

SEQ ID NO: 120 - protein, 9 aa, synthetic construct
QQYSNYHLT

SEQ ID NO: 121 - protein, 6 aa, synthetic construct
QNVGTA

SEQ ID NO: 122 - skipped

SEQ ID NO: 123 - protein, 5 aa, synthetic construct
NYAMT

SEQ ID NO: 124 - protein, 17 aa, synthetic construct
WIDTYSGVPTYADDFKG

SEQ ID NO: 125 - protein, 12 aa, synthetic construct
VGVYGGYDTLDS

SEQ ID NO: 126 - protein, 8 aa, synthetic construct
GYTFTNYA

SEQ ID NO: 127 - protein, 8 aa, synthetic construct
IDTYSGVP

SEQ ID NO: 128 - protein, 14 aa, synthetic construct
ARVGVYGGYDTLDS

SEQ ID NO: 129 - protein, 11 aa, synthetic construct
SASQGISNHLN

SEQ ID NO: 130 - protein, 7 aa, synthetic construct
YTSSLHS

SEQ ID NO: 131 - protein, 9 aa, synthetic construct
QQYNNLPWT

SEQ ID NO: 132 - protein, 6 aa, synthetic construct
QGISNH

SEQ ID NO: 133 - skipped

SEQ ID NO: 134 - protein, 5 aa, synthetic construct
SYGIS

SEQ ID NO: 135 - protein, 17 aa, synthetic construct
EIYPRDDITYYNEKFKG

SEQ ID NO: 136 - protein, 14 aa, synthetic construct
HDYYYVNSVSAMDY

SEQ ID NO: 137 - protein, 8 aa, synthetic construct
GYSFTSYG

SEQ ID NO: 138 - protein, 8 aa, synthetic construct
IYPRDDIT

SEQ ID NO: 139 - protein, 16 aa, synthetic construct
ARHDYYYVNSVSAMDY

SEQ ID NO: 140 - protein, 17 aa, synthetic construct
KSSQSLLYSSNQKNYLA

SEQ ID NO: 141 - protein, 7 aa, synthetic construct
WASTRYS

SEQ ID NO: 142 - protein, 9 aa, synthetic construct
QQYYIYPWT

SEQ ID NO: 143 - protein, 12 aa, synthetic construct
QSLLYSSNQKNY

SEQ ID NO: 144 - skipped

SEQ ID NO: 145 - protein, 5 aa, synthetic construct
DHTFH

SEQ ID NO: 146 - protein, 17 aa, synthetic construct
YIYPRDDNTKYNEKFKG

SEQ ID NO: 147 - protein, 12 aa, synthetic construct
AGYYGSSHAMDY

SEQ ID NO: 148 - protein, 8 aa, synthetic construct
GYMFTDHT

SEQ ID NO: 149 - protein, 8 aa, synthetic construct
IYPRDDNT

SEQ ID NO: 150 - protein, 14 aa, synthetic construct
ARAGYYGSSHAMDY

SEQ ID NO: 151 - protein, 11 aa, synthetic construct
KASQDVSTAVA

SEQ ID NO: 152 - protein, 7 aa, synthetic construct
SASSRYT

SEQ ID NO: 153 - protein, 9 aa, synthetic construct
QQLYSTPWT

SEQ ID NO: 154 - protein, 6 aa, synthetic construct
QDVSTA

SEQ ID NO: 155 - skipped

SEQ ID NO: 156 - protein, 5 aa, synthetic construct
RYWMS

SEQ ID NO: 157 - protein, 17 aa, synthetic construct
EINPDSSTINYAPSLKD

SEQ ID NO: 158 - protein, 11 aa, synthetic construct
FYYANYYAMDY

SEQ ID NO: 159 - protein, 8 aa, synthetic construct
GIDFSRYW

SEQ ID NO: 160 - protein, 8 aa, synthetic construct
INPDSSTI

SEQ ID NO: 161 - protein, 13 aa, synthetic construct
AIFYYANYYAMDY

SEQ ID NO: 162 - protein, 11 aa, synthetic construct
KASQNVDTNVA

SEQ ID NO: 163 - protein, 7 aa, synthetic construct
SASYRFS

SEQ ID NO: 164 - protein, 9 aa, synthetic construct
QQYNGYPFT

SEQ ID NO: 165 - protein, 6 aa, synthetic construct
QNVDTN

SEQ ID NO: 166 - skipped

SEQ ID NO: 167 - protein, 5 aa, synthetic construct
DYWTH

SEQ ID NO: 168 - protein, 17 aa, synthetic construct
YINHGSGYTKYNQKFKD

SEQ ID NO: 169 - protein, 9 aa, synthetic construct
LTGSYYFDY

SEQ ID NO: 170 - protein, 8 aa, synthetic construct
GYTFTDYW

SEQ ID NO: 171 - protein, 8 aa, synthetic construct
INHGSGYT

SEQ ID NO: 172 - protein, 11 aa, synthetic construct
ARLTGSYYFDY

SEQ ID NO: 173 - protein, 10 aa, synthetic construct
SASSSVSYMH

SEQ ID NO: 174 - protein, 7 aa, synthetic construct
DTSKLAS

SEQ ID NO: 175 - protein, 9 aa, synthetic construct
QQWSSNPLT

SEQ ID NO: 176 - protein, 5 aa, synthetic construct
SSVSY

SEQ ID NO: 177 - skipped

SEQ ID NO: 178 - protein, 16 aa, synthetic construct
KSSQSLLYSNAKTYLS

SEQ ID NO: 179 - protein, 17 aa, synthetic construct
YITNAGENTYFPDTVKG

SEQ ID NO: 180 - protein, 17 aa, synthetic construct
WIDTYSGVPTYADSFKG

SEQ ID NO: 181 - protein, 17 aa, synthetic construct
EIYPRDQITYYNEKFKG

SEQ ID NO: 182 - protein, 17 aa, synthetic construct
YIYPRDQNTKYNEKFKG

SEQ ID NO: 183 - protein, 9 aa, synthetic construct
QQYQGYPFT

SEQ ID NO: 184 - protein, 9 aa, synthetic construct
QQYNAYPFT